(19) United States
(12) Patent Application Publication
(10) Pub. No.: US 2021/0009583 A1
(43) Pub. Date: Jan. 14, 2021

(54) DEUTERATED IMIDAZOPYRIDINES

(71) Applicant: UNIVERSITY OF NOTRE DAME DU LAC, SOUTH BEND, IN (US)

(72) Inventors: Rui LIU, Granger, IN (US); Marvin J. MILLER, South Bend, IN (US); Garrett C. MORASKI, Bozeman, MT (US)

(73) Assignee: UNIVERSITY OF NOTRE DAME DU LAC, SOUTH BEND, IN (US)

(21) Appl. No.: 16/980,230

(22) PCT Filed: Mar. 11, 2019
(86) PCT No.: PCT/IB2019/051934

§ 371 Date: Sep. 11, 2020

Related U.S. Application Data
(60) Provisional application No. 62/641,668, filed on Mar. 12, 2018.

(51) Int. Cl. C07D 471/04; A61P 31/06

(52) U.S. Cl. CPC C07D 471/04; A61P 31/06

(57) ABSTRACT

A series of imidazopyridine and pyrazolopyridine compounds is provided in which carbon hydrogen bonds have been replaced with isotopic carbon-deuterium bonds, syntheses thereof, compositions thereof, and methods of using such compounds and compositions. Various embodiments provide methods of killing and/or inhibiting the growth of M. tuberculosis and/or M. avium, and methods of treating, preventing, and/or ameliorating M. tuberculosis, M. avium or other mycobacterial infections in a subject like M. leprae or M. ulcerans.

### RELATED APPLICATIONS

This application claims the benefit of U.S. Application 62/641,668, filed Mar. 12, 2018, the entire contents of which are hereby incorporated by reference.

### GOVERNMENT SUPPORT

This invention was made with government support under Grants No. R01AI054193 and R37AI054193 awarded by the National Institutes of Health. The United States Government has certain rights in the invention.

### FIELD OF THE INVENTION

The invention provides a series of imidazopyridine and pyrazolopyridine compounds in which carbon hydrogen bonds have been replaced with isotopic carbon-deuterium bonds, syntheses thereof, compositions thereof, and methods of using such compounds and compositions. Various embodiments provide methods of killing and/or inhibiting the growth of Mycobacterium tuberculosis and/or Mycobacterium avium, and methods of treating, preventing, and/or ameliorating Mycobacterium tuberculosis, Mycobacterium avium or other mycobacterial infections in a subject, such as Mycobacterium leprae or Mycobacterium ulcerans.

### BACKGROUND

Historically, tuberculosis and other infections caused by strains of mycobacteria have caused more deaths than any other infectious disease. The World Health Organization (WHO) has recently reported that tuberculosis, caused by Mycobacterium tuberculosis is again a leading cause of death (see, Global tuberculosis report 2017, World Health Organization 2017, WHO/HTM/TB/2017.23). The situation is exacerbated by the rapid emergence of multi-drug resistant (MDR), extensive drug resistant (XDR) and now totally drug resistant (TDR) strains as well as coinfections such as TB/HIV. Thus, there is an urgent need to develop new anti-mycobacterial agents.

Various compounds and methods are disclosed in WO 2014/015167, published Jan. 23, 2014; WO 2011/057145, published May 12, 2011, and WO 2017/049321, published Mar. 23, 2017.

### BRIEF DESCRIPTION OF THE FIGURES

FIG. 1 sets out exemplary compounds.

FIG. 2 presents data for Minimum Inhibitory Concentrations (MIC) of compounds screened against Mycobacterium tuberculosis strain H37Rv (H37Rv-Mtb) in two different media (7H12 and GAS) by the MABA assay and against Mycobacterium avium 101 (serotype 1).

FIG. 3 presents data for stability comparison of the non-deuterated analogs

### BRIEF DESCRIPTION OF THE SEVERAL EMBODIMENTS

A series of imidazopyridine and pyrazolopyridine compounds is provided in which carbon hydrogen bonds have been replaced with isotopic carbon-deuterium bonds, syntheses thereof, compositions thereof, and methods of using such compounds and compositions. Various embodiments provide methods of killing and/or inhibiting the growth of M. tuberculosis and/or M. avium, and methods of treating, preventing, and/or ameliorating M. tuberculosis, M. avium or other mycobacterial infections in a subject like M. leprae or M. ulcerans.

Replacement of carbon-hydrogen bonds with isotopic carbon-deuterium bonds results in a kinetic isotope effect. This corresponds to a change in reaction rate of a chemical, and in biological systems, metabolic reactions when one of the atoms in the reactants (therapeutic agent) is replaced by one of its isotopes. Specifically, the kinetic isotope effect (KIE) is the ratio of rate constants for reactions involving bonds associated with the light (k_{L}) and heavy (k_{H}) isotopically substituted reactants.

KIE=k₁/k_{H}

Since deuterium (D) is a heavier isotope of hydrogen (H), carbon-deuterium bonds have higher dissociation energies (are stronger) than carbon-hydrogen bonds. Change of a hydrogen (H) to deuterium (D) represents a 100% increase in mass. Thus, the rate of a reaction involving a C—H bond is 6-10 times faster than the rate of reaction of the corresponding C-D bond.

Accordingly, deuterium incorporation can sometimes significantly alter the metabolic profile of a molecule, thereby resulting in changes in the ratio of parent drug to metabolites and changes in the amounts of metabolites formed. (Harbeson, S. L.; Tung, R. D. “Deuterium Medicinal Chemistry: An New Approach to Drug Discovery and Development”, MedChem News 2014, 2. Also see: S. L. Harbeson and R. D. Tung, (ed. J. E. Macor), in Ann. Rep. Med. Chem., 46, Elsevier, Oxford, U K, 2011, pp. 403-417). Thus substitutions of metabolically labile C—H bonds for stronger C-D bonds can improve pharmacodynamics, tolerability and efficacy of a therapeutic agent. (U.S. Publication No. 2009/0131485, published May 21, 2009; U.S. Pat. No. 6,221,335, issued Apr. 24, 2001; Shao, Liming, and Michael C. Hewitt. “The kinetic isotope effect in the search for deuterated drugs.” Drug News & Perspectives 23.6 (2010): 398-404; U.S. Pat. No. 6,342,507, issued Jan. 29, 2002; Harbeson, Scott L., and Roger D. Tung. “Deuterium medicinal chemistry: A new approach to drug discovery and development.” Medchem News 2 (2014): 8-22). While several deuterated analogs of drugs have been described and found to have improved properties relative to their C—H analogs, the syntheses of the deuterium isotope analogs often involves extensive modifications of the synthetic procedures used to obtain the specifically deuterated compounds. A one-step procedure is also described herein for conversion of multiple C—H bonds to C-D bonds in imidazopyridine and pyrazolopyridine molecules under development for treatment of mycobacterial infections.

Imidazopyridine and pyrazolopyridines are potent anti-mycobacterial agents currently under development for the treatment of tuberculosis (Mtb) and non-tuberculosis mycobacterial infections (NTMs). (Moraski, Garrett C., et al. “Advent of imidazo [1,2-a] pyridine-3-carboxamides with potent multi- and extended drug resistant antituberculosis activity.” ACS medicinal chemistry letters 2.6 (2011): 466-470; Moraski, Garrett C., et al. “Advancement of Imidazo [1,2-a] pyridines with Improved Pharmacokinetics and nM Activity vs. Mycobacterium tuberculosis.” ACS medicinal chemistry letters 4.7 (2013): 675-679; Pethe, Kevin, et al. “Discovery of Q203, a potent clinical candidate for the treatment of tuberculosis.” Nature medicine 19.9 (2013): 1157-1160; Abrahams, Katherine A., et al. “Identification of novel imidazo [1,2-a] pyridine inhibitors targeting M. tuberculosis QcrB.” PloS one 7.12 (2012): e52951; Kang, Sunhee, et al. “Lead optimization of a novel series of imidazo [1,2-a]pyridine amides leading to a clinical candidate (Q203) as a multi- and extensively-drug-resistant anti-tuberculosis agent.” Journal of Medicinal Chemistry 57.12 (2014): 5293-5305); Pyrazolo[1,5-a]pyridine Inhibitor of the Respiratory Cytochrome bec Complex for the Treatment of Drug-Resistant Tuberculosis, ACS Infect. Dis. 2019, 5, 239-249.

In one aspect, the invention provides novel deuterated imidazopyridine compounds of general formulas (A) and (A-Deut):

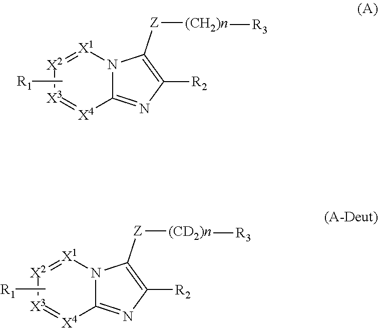

or salts thereof;

wherein:

X¹, X², X³, and X⁴ are independently C-D, CR₄, or N or a combination thereof;

Z is independently —C(O)NH—, —NHC(O)—; —C(O)O—, —C(O)C(O)—, —CH₂C(O)—, —CD₂C(O)—, —C(O)CH₂—, —C(O)CD₂-, or —NH—C(O)NH—;

n is an integer of 0 to 4;

each R₁ is independently D, CD₃, —OCD₃, Cl, Br, CF₃, SF₅, SF₃, F, —P(O)(CH₃)₂, SO₂CH₃, alkyl, deuterated alkyl, heterocycle, deuterated heterocycle, methoxy, alkoxy, deuterated alkoxy, aryl, deuterated aryl, heteroaryl, deuterated heteroaryl, amine, substituted amine, or deuterated amine;

R₂ is independently CD₃, CH₂CD₃, CH₃, —CD₂CD₃, ethyl, extended alkyl, deuterated alkyl, heterocycle, deuterated heterocycle, alkoxy, deuterated alkoxy, aryl, deuterated aryl, heteroaryl, deuterated heteroaryl, deuterated amine, cycloalkyl, deuterated cycloalkyl;

R₃ is independently H, D, alkyl, deuterated alkyl, alkoxy, deuterated alkoxy, amino, alkyl amino, deuterated alkyl amino, cycloalkyl, deuterated cycloalkyl, heterocycle, deuterated heterocycle, aryl, deuterated aryl, aryloxy, or deuterated aryloxy; and

each R₄ is independently H, D, alkyl, deuterated alkyl, alkoxy, deuterated alkoxy, amino, alkyl amino, deuterated alkyl amino, cycloalkyl, deuterated cycloalkyl, heterocycle, deuterated heterocycle, aryl, deuterated aryl, aryloxy, deuterated aryloxy, halo, SF₅, SF₃, —P(O)(CH₃)₂, SO₂CH₃, or R₁;

and wherein in (A), at least one or more of R₁, R₂, R₃, or R₄ is deuterated.

In another aspect, the invention provides novel deuterated pyrazolopyridine compounds of general formulas (B) and (B-Deut):

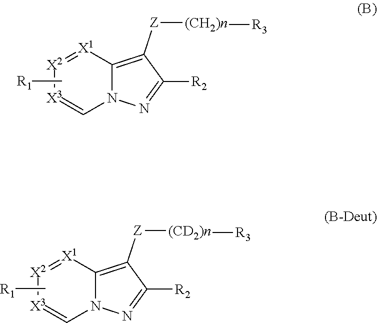

or salts thereof;

wherein:

X¹, X², and X³ are independently C-D, CR₄, or N or a combination thereof;

Z is independently —C(O)NH—, —NHC(O)—; —C(O)O—, —C(O)C(O)—, —CH₂C(O)—, —CD₂C(O)—, —C(O)CH₂—, —C(O)CD₂-, or —NH—C(O)NH—;

n is an integer of 0 to 4;

each R₁ is independently D, CD₃, —OCD₃, Cl, Br, CF₃, SF₅, SF₃, F, —P(O)(CH₃)₂, SO₂CH₃, alkyl, deuterated alkyl, heterocycle, deuterated heterocycle, methoxy, alkoxy, deuterated alkoxy, aryl, deuterated aryl, heteroaryl, deuterated heteroaryl, amine, substituted amine, or deuterated amine;

R₂ is independently CD₃, CH₂CD₃, CH₃, —CD₂CD₃, ethyl, extended alkyl, deuterated alkyl, heterocycle, deuterated heterocycle, alkoxy, deuterated alkoxy, aryl, deuterated aryl, heteroaryl, deuterated heteroaryl, deuterated amine, cycloalkyl, deuterated cycloalkyl;

R₃ is independently H, D, alkyl, deuterated alkyl, alkoxy, deuterated alkoxy, amino, alkyl amino, deuterated alkyl amino, cycloalkyl, deuterated cycloalkyl, heterocycle, deuterated heterocycle, aryl, deuterated aryl, aryloxy, or deuterated aryloxy; and

each R₄ is independently H, D, alkyl, deuterated alkyl, alkoxy, deuterated alkoxy, amino, alkyl amino, deuterated alkyl amino, cycloalkyl, deuterated cycloalkyl, heterocycle, deuterated heterocycle, aryl, deuterated aryl, aryloxy, deuterated aryloxy, halo, SF₅, SF₃, —P(O)(CH₃)₂, SO₂CH₃, or R₁;

and wherein in (B), at least one or more of R₁, R₂, R₃, or R₄ is deuterated.

In another aspect, the invention also provides a pharmaceutically acceptable salt of any one or more of the compounds described herein.

In another aspect, the invention also provides for a composition, which includes any one or more of the compounds described herein, and a pharmaceutically acceptable carrier or diluent.

In another aspect, the invention also provides for a method of treating a tuberculosis mycobacterial infection or non-tuberculosis mycobacterial infection in a subject, which includes administering to said subject any one or more of the compounds or compositions described herein.

In another aspect, the invention also provides for a method of killing or inhibiting the growth of M. tuberculosis, M. avium, M. leprae, or M. ulcerans, or a combination thereof, in a subject, comprising administering to said subject any one or more of the compounds or compositions described herein.

In another aspect, the invention also provides for any one or more of the compounds or compositions described herein for treatment of a tuberculosis mycobacterial infection or non-tuberculosis mycobacterial infection.

### DETAILED DESCRIPTION OF THE SEVERAL EMBODIMENTS

In any embodiment, an alkyl, cycloalkyl, heterocycle, aryl, aryloxy, heteroaryl, alkoxy, or amine of an R group (e.g., R₁, R₂, R₃, or R₄) can be unsubstituted, or alternatively, substituted with one to five substituents, such as one or more of (C₁-C₆)alkyl, (C₁-C₆)alkene, (C₁-C₆)alkyne, epoxide, oxo, alkyl carboxylate, alkoxy, carbaldehyde, —P(O)(CH₃)₂, SO₂CH₃, halo, OH, SF₅, SF₃, CN, NO₂, or SH groups, or a combination thereof.

In any embodiment, n is 0.

In any embodiment, n is 1.

In any embodiment, n is 2.

In any embodiment, n is 3.

In any embodiment, n is 4.

In embodiments, R₃ is phenyl, pyridyl, indolyl, dihydrobenzofuranyl, or benzo[d]oxazolyl, where each R₃ can be unsubstituted, or substituted as described herein.

In embodiments, R₃ is phenyl or pyridyl substituted with one, two, or three alkyl, alkoxy, halo, trifluoromethyl, trifluoromethoxy, methylamino, dimethylamino, phenyl, phenyloxy, morpholino, thiomorpholino, piperazinyl, piperidinyl, imidazolyl, diazinyl, triazinyl, or pyrolidinyl groups.

In embodiments, R₁ is (C₁-C₆)alkyl, halo, or trifluoromethyl.

In embodiments, R₂ is (C₁-C₆)alkyl or trifluoromethyl.

In embodiments, X¹ is CH, Z is —C(═O)NH—, or —C(═O)O—.

In embodiments, Z is —C(═O)NH—.

In embodiments, Z is —C(═O)O—.

In embodiments, Z is —C(═O)C(═O)—.

In embodiments, Z is —CH₂C(═O)—, —C(═O)CH₂—, or —NH—C(═O)NH—.

In embodiments, R₁ is methyl, trifluoromethyl, chloro, or fluoro.

In embodiments, R₂ is methyl, trifluoromethyl, or ethyl.

In embodiments, Z is —C(═O)NH—, n is an integer of 1 to 4, R₁ is methyl, trifluoromethyl, chloro, or fluoro, and R₂ is methyl, trifluoromethyl, or ethyl.

In embodiments, for example, R₃ can be:

(a) OR₄ or NHR₄;

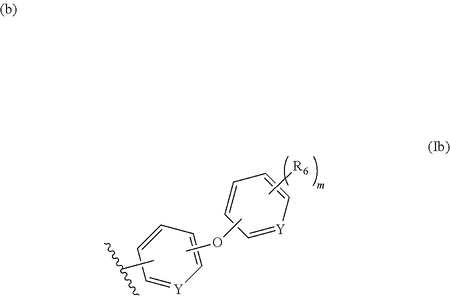

wherein each Y is independently CH or N; R₆ is H, CF₃, OCF₃, SF₅, SF₃, —P(O)(CH₃)₂, SO₂CH₃, halo, methylsulfone, alkoxy, amine, or nitrile; and m is an integer of 1 to 4;

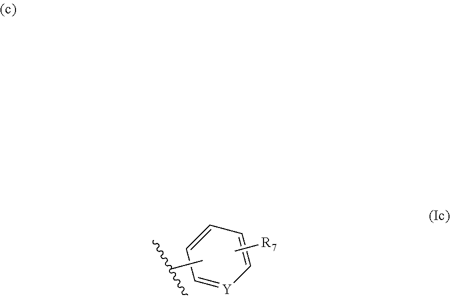

wherein Y is CH or N; and R₇ is a —P(O)(CH₃)₂, SO₂CH₃, or heterocycle, for example, an optionally substituted furan, thiophene, imidazole, oxazole, isoxazole oxazoline, oxadiazole, thiadiazole, thiazole, thiazoline, triazole, pyridine, pyrazine, pyrazole, diketopiperazine, quinoline, isoquinoline, or oxazolidinone;

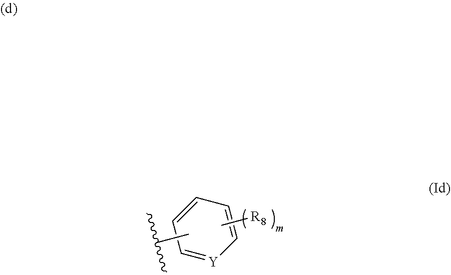

wherein Y is CH or N; and R₈ is H, CF₃, OCF₃, SF₅, SF₃, —P(O)(CH₃)₂, SO₂CH₃, halo, methylsulfone, nitrile, or optionally substituted alkoxy, amine, phenyl, or heterocycle; and m is an integer of 0 to 3;

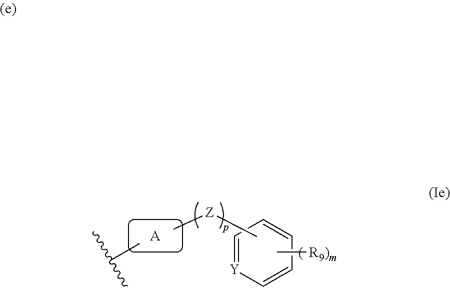

wherein A is a heterocycle, for example, an optionally substituted furan, thiophene, imidazole, oxazole, isoxazole, oxazoline, oxadiazole, thiadiazole, thiazole, thiazoline, triazole, pyridine, pyrazine, diketopiperazine, quinoline, isoquinoline, benzimidazole, benzoxazole, benzothiazole, pyrazole or oxazolindinone; R₉ is CF₃, OCF₃, SF₅, SF₃, —P(O)(CH₃)₂, SO₂CH₃, halo, methylsulfone, alkoxy, amine or nitrile; Y is CH or N; Z is O, CH₂, CH₂O, C(O), C(O)CH₂; m is an integer of 0 to 5; and p is an integer of 0 to 4;

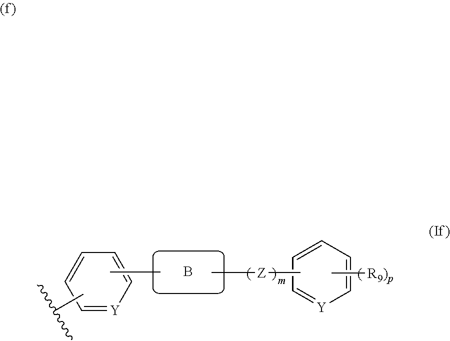

wherein B is a heterocycle, for example, an optionally substituted azetidine, piperazine, homopiperzine, piperidine or azepane; R₉ is H, CF₃, OCF₃, SF₅, SF₃, —P(O)(CH₃)₂, SO₂CH₃, halo, methylsulfone, alkoxy, amine, or nitrile; Y is CH or N; Z is O, CH₂, CH₂O, C(O), C(O)CH₂; m is an integer of 0 to 2; and p is an integer of 0 to 4;

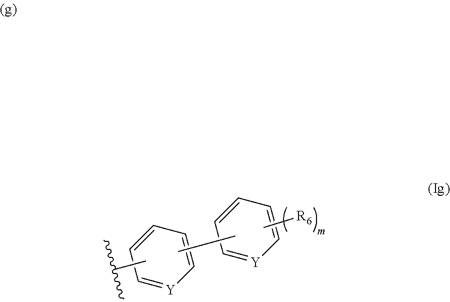

wherein each Y is independently CH or N; R₆ is CF₃, OCF₃, SF₅, SF₃, —P(O)(CH₃)₂, SO₂CH₃, halo, methylsulfone, alkoxy, amine or nitrile; and m is an integer of 0 to 4;

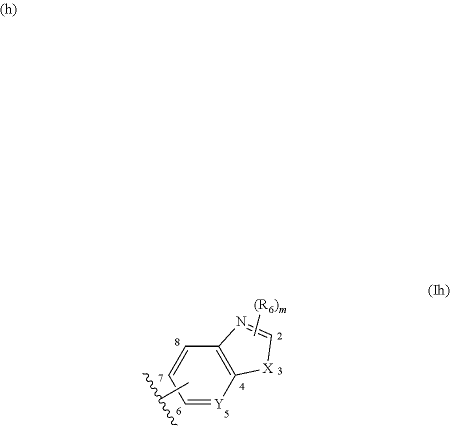

wherein the structure (Ih) is connected to the structure of Formulas A or A-Deut or B or B-Deut at position 2, 6, or 7 of (Ih); R₆ when present is located at position 2, 6, or 7 of (Ih), or a combination thereof, provided that structure (Ih) is not connected to the structure of Formulas A or A-Deut or B or B-Deut at the same position on (Ih); X is CH₂, NH, NR₄′, S, SO, SO₂, or O; Y is CH or N; R₄′ is H, D, alkyl, deuterated alkyl, alkoxy, deuterated alkoxy, amino, alkyl amino, deuterated alkyl amino, cycloalkyl, deuterated cycloalkyl, heterocycle, deuterated heterocycle, aryl, deuterated aryl, aryloxy, deuterated aryloxy, halo, SF₅, SF₃, or —P(O)(CH₃)₂, SO₂CH₃, R₆ is CF₃, OCF₃, SF₅, SF₃, —P(O)(CH₃)₂, SO₂CH₃, halo, methylsulfone, alkoxy, amine or nitrile; and m is an integer of 0 to 3;

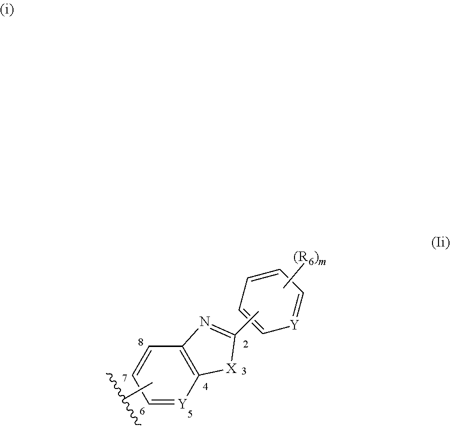

wherein the structure (Ii) is connected to the structure of Formulas A or A-Deut or B or B-Deut at position 6 or 7 of (Ii); R₆ when present is located at position 6 or 7 of (Ii), or on the pendant C₅H₄Y ring as shown in (Ii), or a combination thereof on (Ii), provided that structure (Ii) is not connected to the structure of Formulas A or A-Deut or B or B-Deut at the same position on (Ii); X is CH₂, NH, NR₄′, S, SO, SO₂, or O; Y is CH or N; R₄′ is H, D, alkyl, deuterated alkyl, alkoxy, deuterated alkoxy, amino, alkyl amino, deuterated alkyl amino, cycloalkyl, deuterated cycloalkyl, heterocycle, deuterated heterocycle, aryl, deuterated aryl, aryloxy, deuterated aryloxy, halo, SF₅, SF₃, or —P(O)(CH₃)₂, SO₂CH₃, R₆ is CF₃, OCF₃, SF₅, SF₃, —P(O)(CH₃)₂, SO₂CH₃, halo, methylsulfone, alkoxy, amine or nitrile; and m is an integer of 0 to 3.

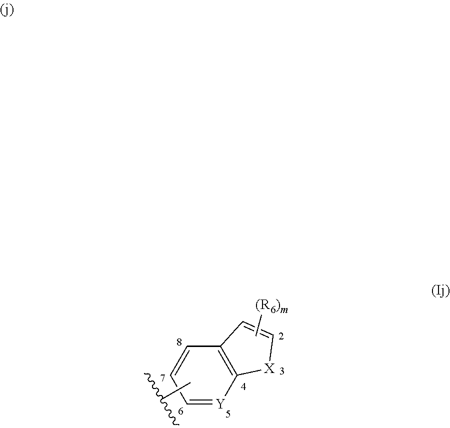

wherein the structure (Ij) is connected to the structure of Formulas A or A-Deut or B or B-Deut at position 2, 6, or 7 of (Ij); R₆ when present is located at position 2, 6, or 7 of (Ij), or a combination thereof, provided that structure (Ij) is not connected to the structure of Formulas A or A-Deut or B or B-Deut at the same position on (Ij); X is CH₂, NH, NR₄′, S, SO, SO₂, or O; Y is CH or N; R₄′ is H, D, alkyl, deuterated alkyl, alkoxy, deuterated alkoxy, amino, alkyl amino, deuterated alkyl amino, cycloalkyl, deuterated cycloalkyl, heterocycle, deuterated heterocycle, aryl, deuterated aryl, aryloxy, deuterated aryloxy, halo, SF₅, SF₃, or —P(O)(CH₃)₂, SO₂CH₃, R₆ is CF₃, OCF₃, SF₅, SF₃, —P(O)(CH₃)₂, SO₂CH₃, halo, methylsulfone, alkoxy, amine or nitrile; and m is an integer of 0 to 3;

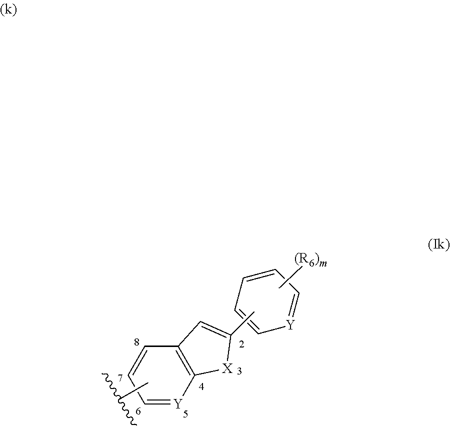

wherein the structure (Ik) is connected to the structure of Formulas A or A-Deut or B or B-Deut at position 6 or 7 of (Ik); R₆ when present is located at position 6 or 7 of (Ik), or on the pendant C₅H₄Y ring as shown in (Ik), or a combination thereof on (Ik), provided that structure (Ik) is not connected to the structure of Formulas A or A-Deut or B or B-Deut at the same position on (Ik); X is CH₂, NH, NR₄′, S, SO, SO₂, or O; Y is CH or N; R₆ is CF₃, OCF₃, SF₅, SF₃, —P(O)(CH₃)₂, SO₂CH₃, halo, methylsulfone, alkoxy, amine or nitrile; and m is an integer of 0 to 3.

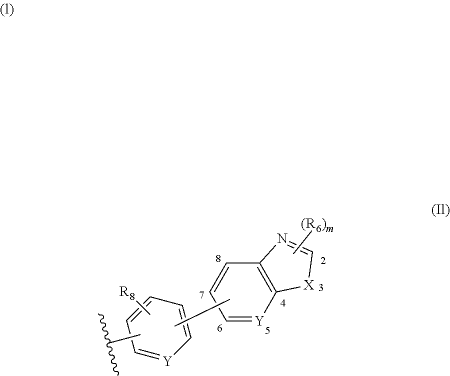

wherein R₆ when present is located at position 2, 6, or 7 of (II), or a combination thereof; X is CH₂, NH, NR₄′, S, SO, SO₂, or O; each Y is independently CH or N; R₄′ is H, D, alkyl, deuterated alkyl, alkoxy, deuterated alkoxy, amino, alkyl amino, deuterated alkyl amino, cycloalkyl, deuterated cycloalkyl, heterocycle, deuterated heterocycle, aryl, deuterated aryl, aryloxy, deuterated aryloxy, halo, SF₅, SF₃, —P(O)(CH₃)₂, or SO₂CH₃; R₆ is CF₃, OCF₃, SF₅, SF₃, —P(O)(CH₃)₂, SO₂CH₃, halo, methylsulfone, alkoxy, amine or nitrile;

R₈ is H, CF₃, OCF₃, SF₅, SF₃, —P(O)(CH₃)₂, SO₂CH₃, halo, methylsulfone, nitrile, optionally substituted alkoxy, amine, phenyl, or heterocycle;

and m is an integer of 0 to 3;

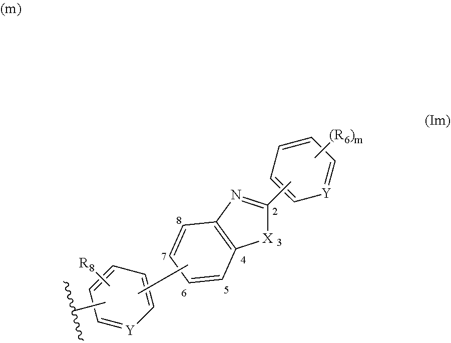

wherein R₆ when present is located at position 6 or 7 of (Im), or on the pendant C₅H₄Y ring as shown in (Im), or a combination thereof on (Im); X is CH₂, NH, NR₄′, S, SO, SO₂, or O; each Y is independently CH or N; R₆ is CF₃, OCF₃, SF, SF₃, —P(O)(CH₃)₂, SO₂CH₃, halo, methylsulfone, alkoxy, amine or nitrile; R₄′ is H, D, alkyl, deuterated alkyl, alkoxy, deuterated alkoxy, amino, alkyl amino, deuterated alkyl amino, cycloalkyl, deuterated cycloalkyl, heterocycle, deuterated heterocycle, aryl, deuterated aryl, aryloxy, deuterated aryloxy, halo, SF₅, SF₃, —P(O)(CH₃)₂, or SO₂CH₃; R₈ is H, CF₃, OCF₃, SF₅, SF₃, —P(O)(CH₃)₂, SO₂CH₃, halo, methylsulfone, nitrile, optionally substituted alkoxy, amine, phenyl, or heterocycle; and m is an integer of 0 to 3;

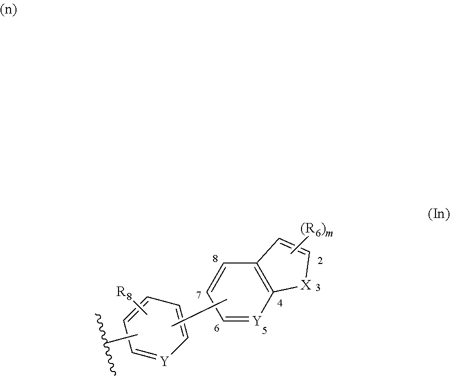

wherein R₆ when present is located at position 2, 6, or 7 of (In), or a combination thereof; X is CH₂, NH, NR₄′, S, SO, SO₂, or O; each Y is independently CH or N; R₄′ is H, D, alkyl, deuterated alkyl, alkoxy, deuterated alkoxy, amino, alkyl amino, deuterated alkyl amino, cycloalkyl, deuterated cycloalkyl, heterocycle, deuterated heterocycle, aryl, deuterated aryl, aryloxy, deuterated aryloxy, halo, SF₅, SF₃, —P(O)(CH₃)₂, or SO₂CH₃; R₆ is CF₃, OCF₃, SF₅, SF₃, —P(O)(CH₃)₂, SO₂CH₃, halo, methylsulfone, alkoxy, amine or nitrile; R₈ is H, CF₃, OCF₃, SF₅, SF₃, —P(O)(CH₃)₂, SO₂CH₃, halo, methylsulfone, nitrile, optionally substituted alkoxy, amine, phenyl, or heterocycle; and m is an integer of 0 to 3;

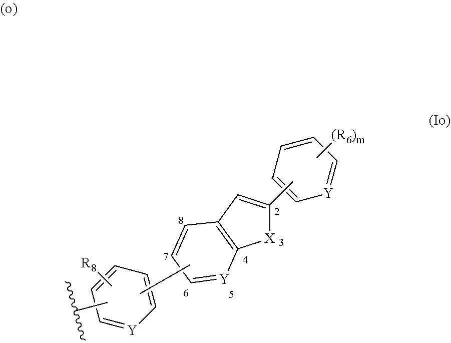

wherein R₆ when present is located at position 6 or 7 of (Ik), or on the pendant C₅H₄Y ring as shown in (Ik), or a combination thereof on (Ik); X is CH₂, NH, NR₄′, S, SO, SO₂, or O; each Y is independently CH or N; R₄′ is H, D, alkyl, deuterated alkyl, alkoxy, deuterated alkoxy, amino, alkyl amino, deuterated alkyl amino, cycloalkyl, deuterated cycloalkyl, heterocycle, deuterated heterocycle, aryl, deuterated aryl, aryloxy, deuterated aryloxy, halo, SF₅, SF₃, —P(O)(CH₃)₂, or SO₂CH₃; R₆ is CF₃, OCF₃, SF₅, SF₃, —P(O)(CH₃)₂, SO₂CH₃, halo, methylsulfone, alkoxy, amine or nitrile; R₈ is H, CF₃, OCF₃, SF, SF₃, —P(O)(CH₃)₂, SO₂CH₃, halo, methylsulfone, nitrile, optionally substituted alkoxy, amine, phenyl, or heterocycle; and m is an integer of 0 to 3;

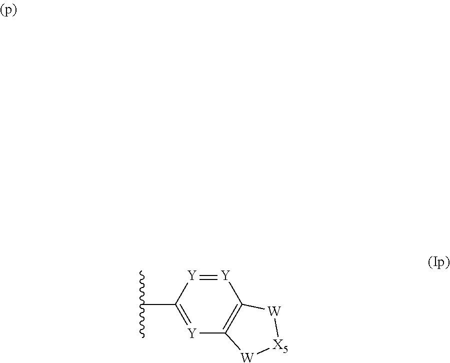

wherein X₅ is CH₂, CHR₈, CR₈R₈, S, SO, SO₂, O, NH, NR₈,

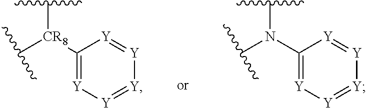

wherein each R₈ is H, CF₃, OCF₃, SF₅, SF₃, —P(O)(CH₃)₂, SO₂CH₃, halo, methylsulfone, nitrile, optionally substituted alkoxy, amine, phenyl, or heterocycle;

wherein each Y is independently CH, CR₉, or N; wherein at most two Ys in any one ring are N; each R₉ is H, CF₃, OCF₃, SF, SF₃, —P(O)(CH₃)₂, SO₂CH₃, halo, methylsulfone, alkoxy, amine, or nitrile;

wherein each W is independently CH₂, CHR₁₀, CR₁₀R₁₀, NH, NR₁₀, S, SO, SO₂, or O; each R₁₀ is independently H, alkyl, t-butyl, isopropoxy, CF₃, OCF₃, SF₅, SF₃, halo, F, methylsulfone, alkoxy, amine, or nitrile;

and wherein any of the alkyl, t-butyl, alkoxy, isopropoxy in any R group is each independently substituted or unsubstituted, branched or unbranched, or any combination thereof;

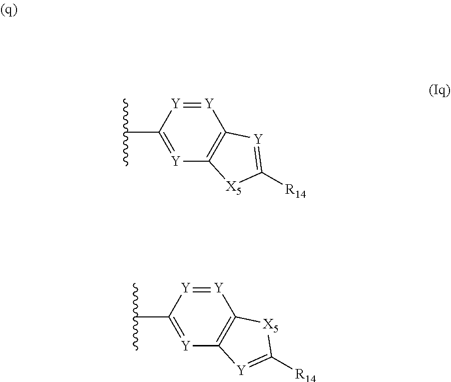

wherein X₅ is CH₂, CHR₈, CR₈R₈, S, SO, SO₂, O, NH, NR₈;

wherein each R₈ is H, CF₃, OCF₃, SF₅, SF₃, —P(O)(CH₃)₂, SO₂CH₃, halo, methylsulfone, nitrile, optionally substituted alkoxy, amine, phenyl, or heterocycle;

wherein R₁₄ is independently H, alkyl, t-butyl, isopropoxy, CF₃, OCF₃, SF₅, SF₃, halo, F, methylsulfone, alkoxy, amine, nitrile, or

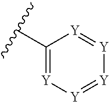

wherein each Y is independently CH, CR₉, or N; wherein at most two Ys in any one ring are N; each R₉ is H, CF₃, OCF₃, SF₅, SF₃, —P(O)(CH₃)₂, SO₂CH₃, halo, methylsulfone, alkoxy, amine, or nitrile;

and wherein any of the alkyl, t-butyl, alkoxy, isopropoxy in any R group is each independently substituted or unsubstituted, branched or unbranched, or any combination thereof;

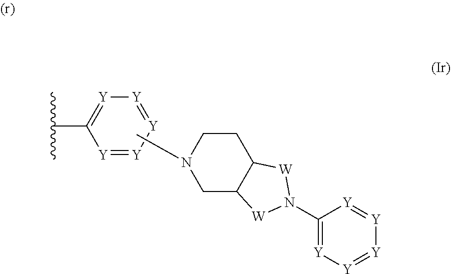

wherein each Y is independently CH, CR₉, or N; wherein at most two Ys in any one ring are N; each R₉ is H, CF₃, OCF₃, SF₅, SF₃, —P(O)(CH₃)₂, SO₂CH₃, halo, methylsulfone, alkoxy, amine, or nitrile;

wherein each W is independently CH₂, CHR₁₀, CR₁₀R₁₀, NH, NR₁₀, S, SO, SO₂, or O; each R₁₀ is independently H, alkyl, t-butyl, isopropoxy, CF₃, OCF₃, SF₅, SF₃, halo, F, methylsulfone, alkoxy, amine, or nitrile;

and wherein any of the alkyl, t-butyl, alkoxy, isopropoxy in any R group is each independently substituted or unsubstituted, branched or unbranched, or any combination thereof;

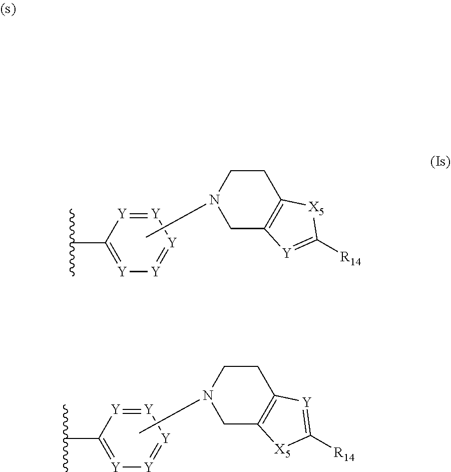

wherein X₅ is CH₂, CHR₈, CR₈R₈, S, SO, SO₂, O, NH, NR₈; wherein each R₈ is H, CF₃, OCF₃, SF₅, SF₃, —P(O)(CH₃)₂, SO₂CH₃, halo, methylsulfone, nitrile, optionally substituted alkoxy, amine, phenyl, or heterocycle;

wherein R₁₄ is independently H, alkyl, t-butyl, isopropoxy, CF₃, OCF₃, SF₅, SF₃, halo, F, methylsulfone, alkoxy, amine, nitrile, or

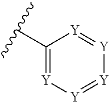

and

wherein each Y is independently CH, CR₉, or N; wherein at most two Ys in any one ring are N; each R₉ is H, CF₃, OCF₃, SF, SF₃, —P(O)(CH₃)₂, SO₂CH₃, halo, methylsulfone, alkoxy, amine, or nitrile;

and wherein any of the alkyl, t-butyl, alkoxy, isopropoxy in any R group is each independently substituted or unsubstituted, branched or unbranched, or any combination thereof

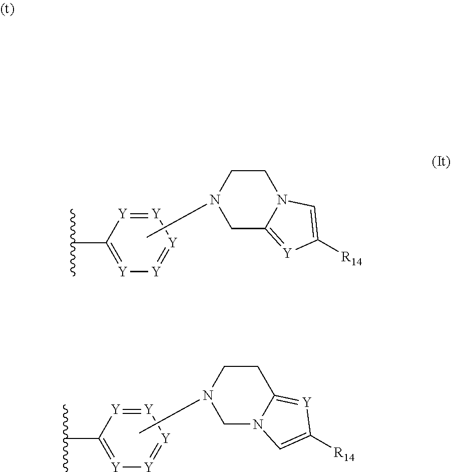

wherein R₁₄ is independently H, alkyl, t-butyl, isopropoxy, CF₃, OCF₃, SF₅, SF₃, halo, F, methylsulfone, alkoxy, amine, nitrile, or

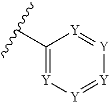

and

wherein each Y is independently CH, CR₉, or N; wherein at most two Ys in any one ring are N; each R₉ is H, CF₃, OCF₃, SF₅, SF₃, —P(O)(CH₃)₂, SO₂CH₃, halo, methylsulfone, alkoxy, amine, or nitrile;

and wherein any of the alkyl, t-butyl, alkoxy, isopropoxy in any R group is each independently substituted or unsubstituted, branched or unbranched, or any combination thereof.

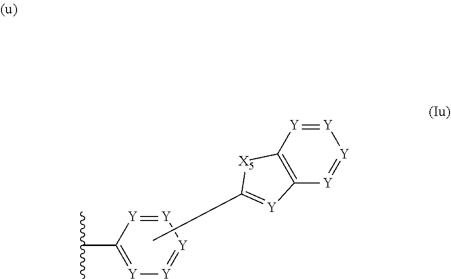

wherein X₅ is CH₂, CHR₈, CR₈R₈, S, SO, SO₂, O, NH, NR₈; wherein each R₈ is H, CF₃, OCF₃, SF₅, SF₃, —P(O)(CH₃)₂, SO₂CH₃, halo, methylsulfone, nitrile, optionally substituted alkoxy, amine, phenyl, or heterocycle; and

wherein each Y is independently CH, CR₉, or N; wherein at most two Ys in any one ring are N; each R₉ is H, CF₃, OCF₃, SF₅, SF₃, —P(O)(CH₃)₂, SO₂CH₃, halo, methylsulfone, alkoxy, amine, or nitrile; or

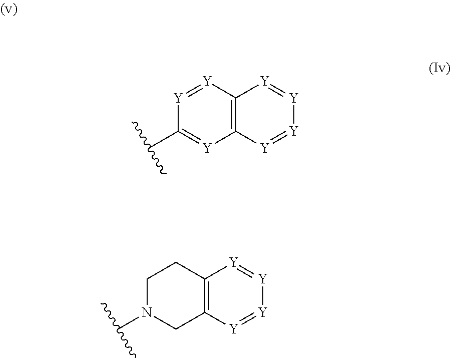

wherein each Y is independently CH, CR9, or N; wherein at most two Ys in any one ring are N; each R9 is independently H, alkyl, t-butyl, isopropoxy, CF₃, OCF₃, SF₅, SF₃, halo, methylsulfone, alkoxy, amine, or nitrile.

Some examples of (A) and (A-Deut):

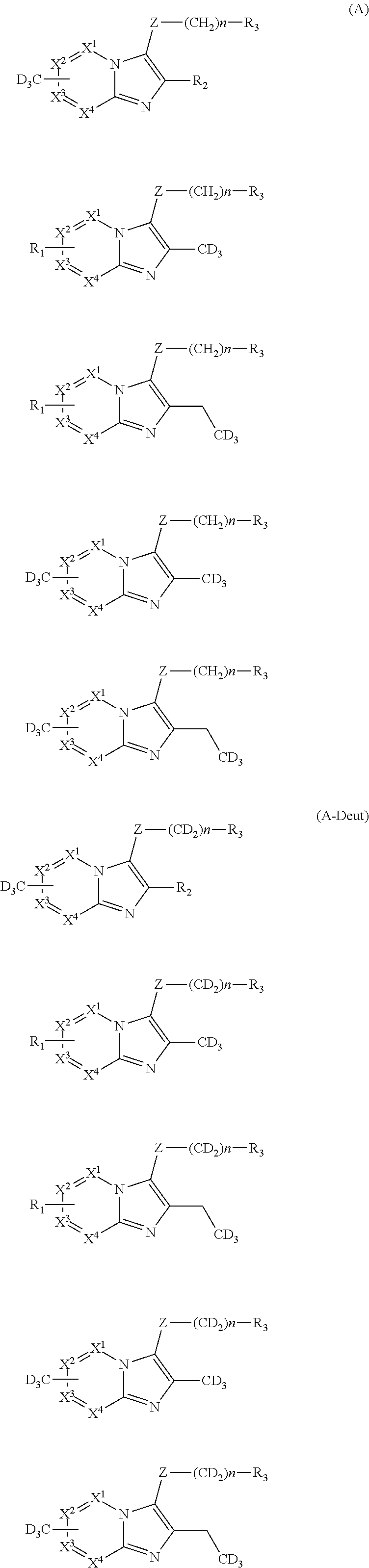

Some examples of (B) and (B-Deut):

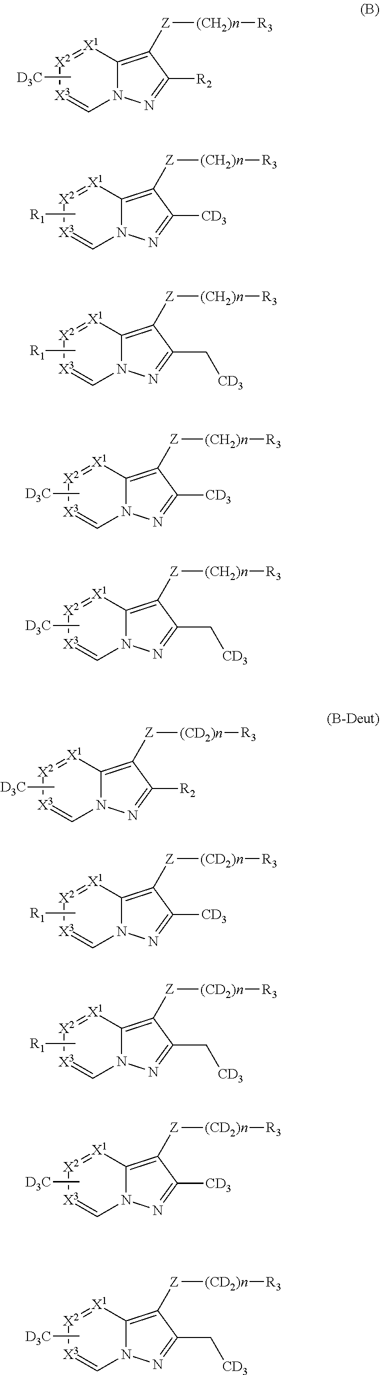

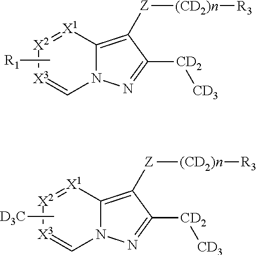

Other examples of (A) and (A-Deut):

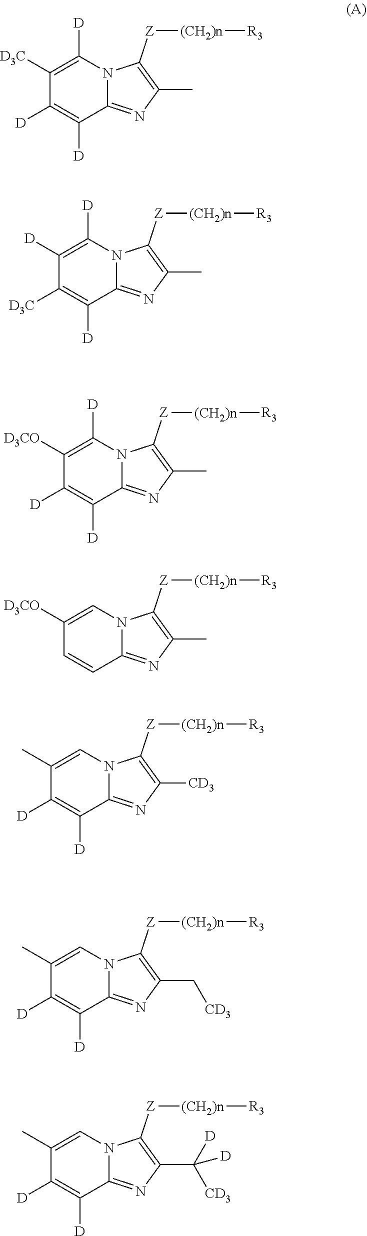

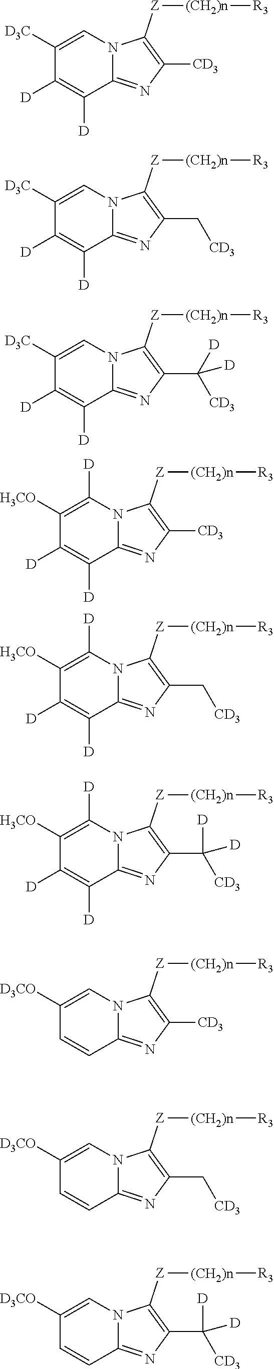

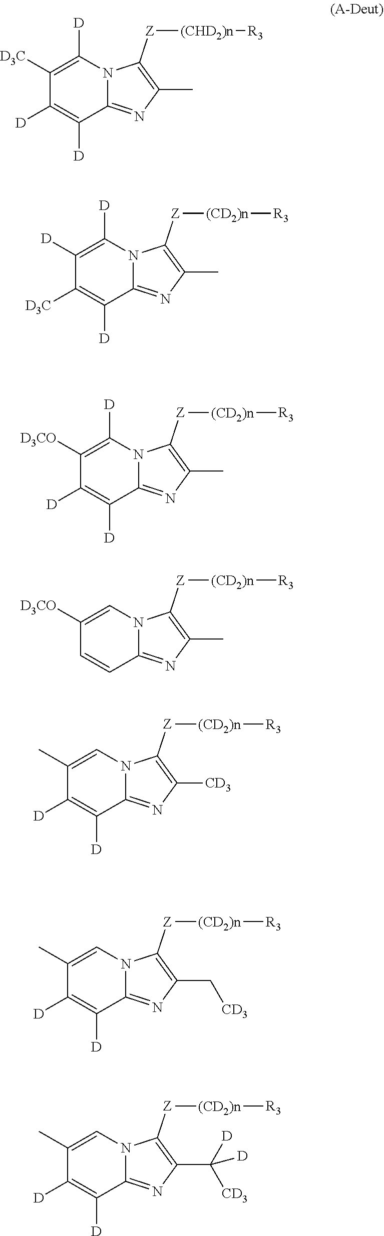

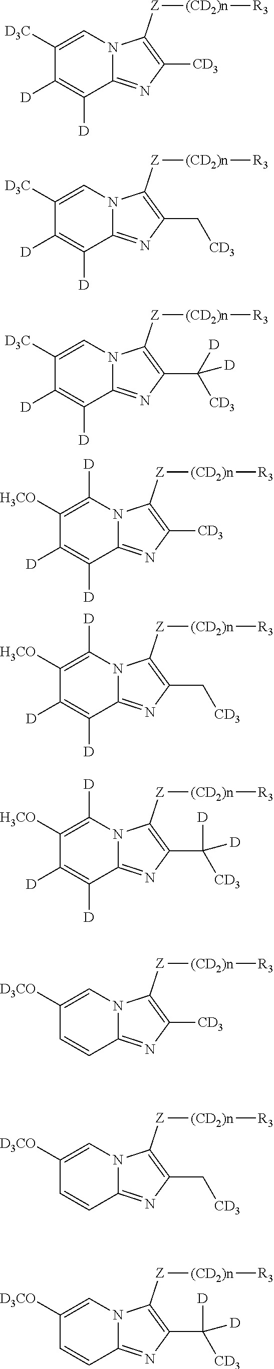

Other examples of (B) and (B-Deut):

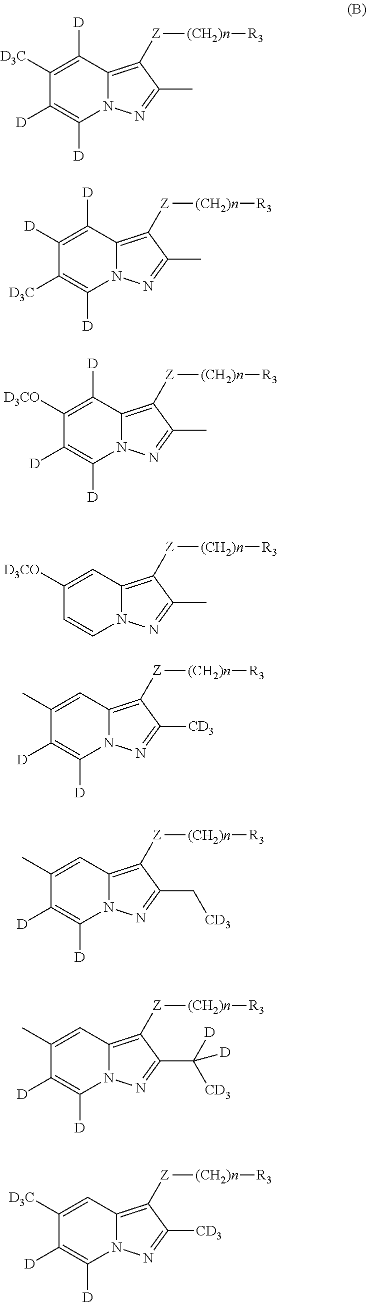

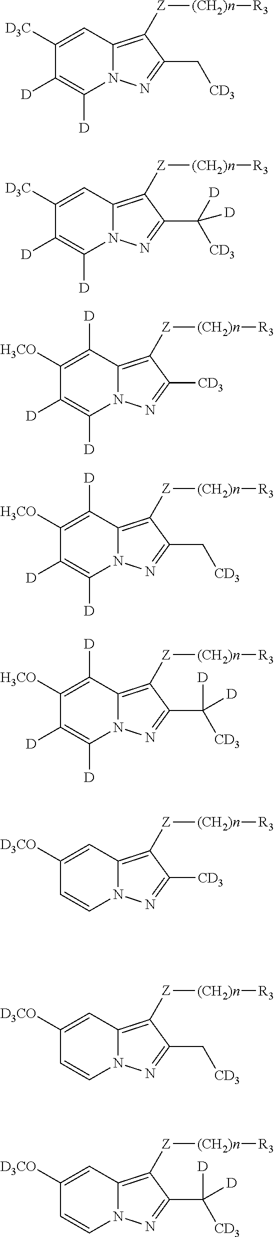

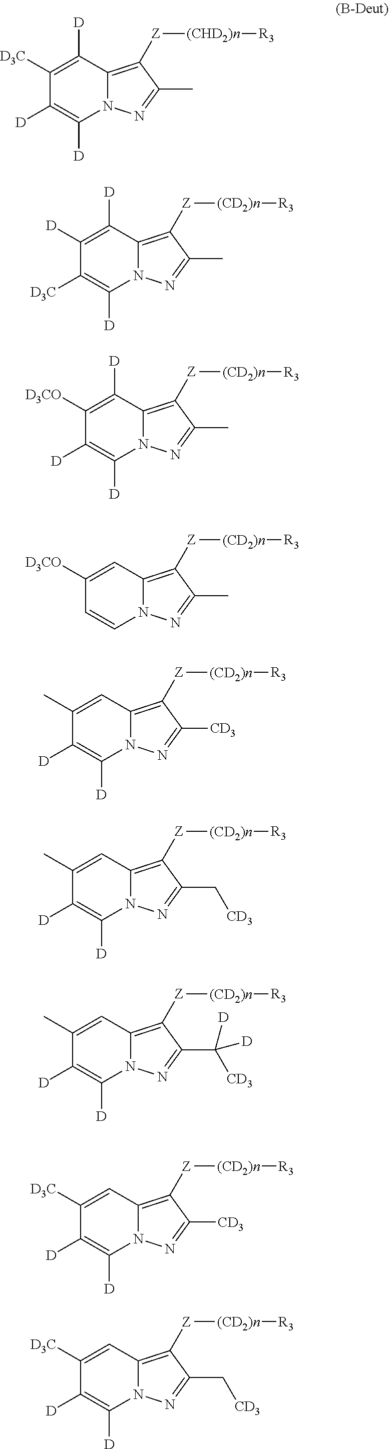

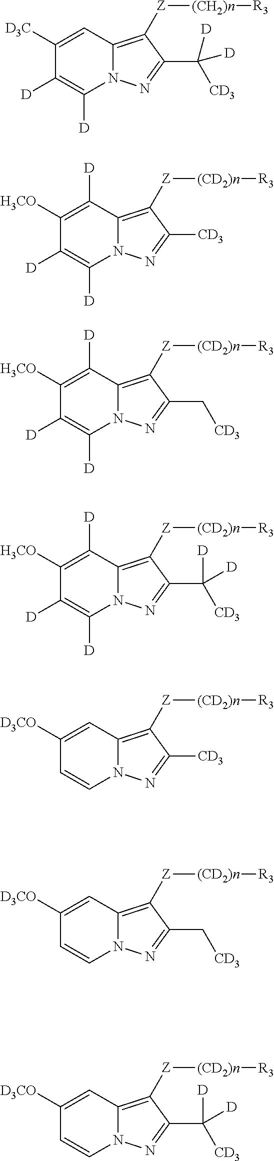

In any compound, in the portion of the structure:

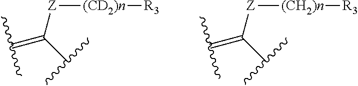

R₃ may be any of the following:

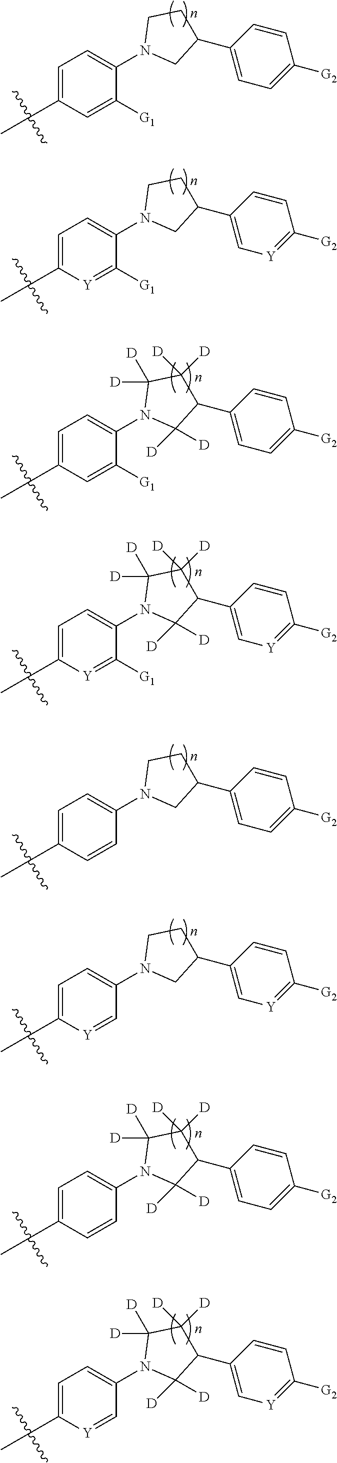

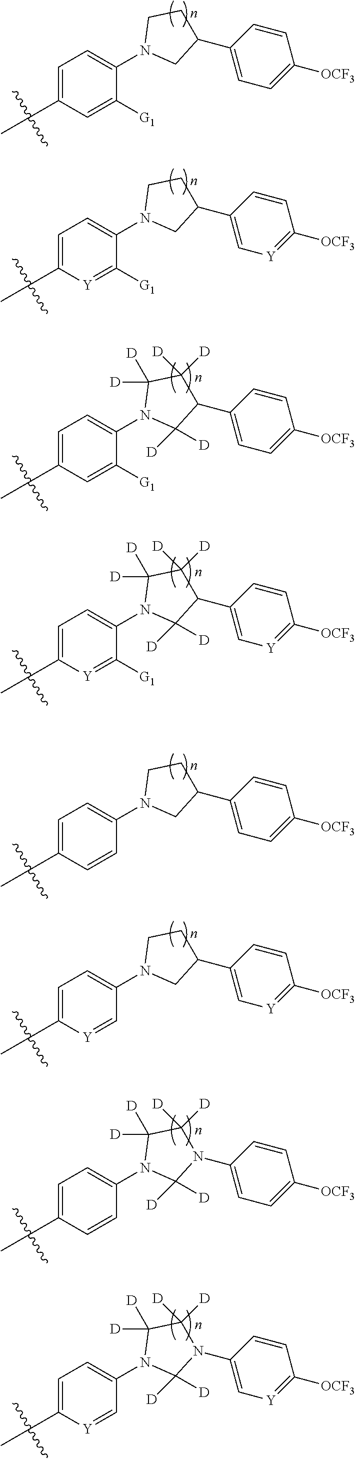

Or any of the following:

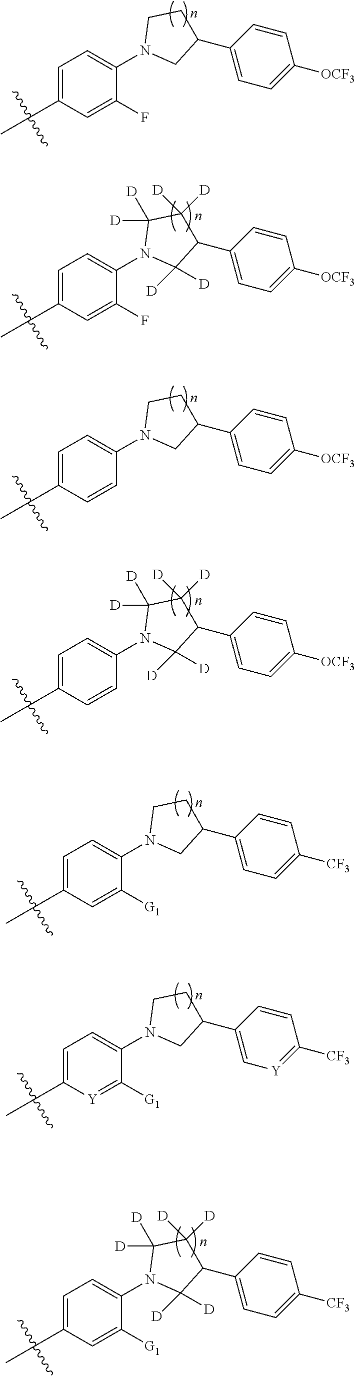

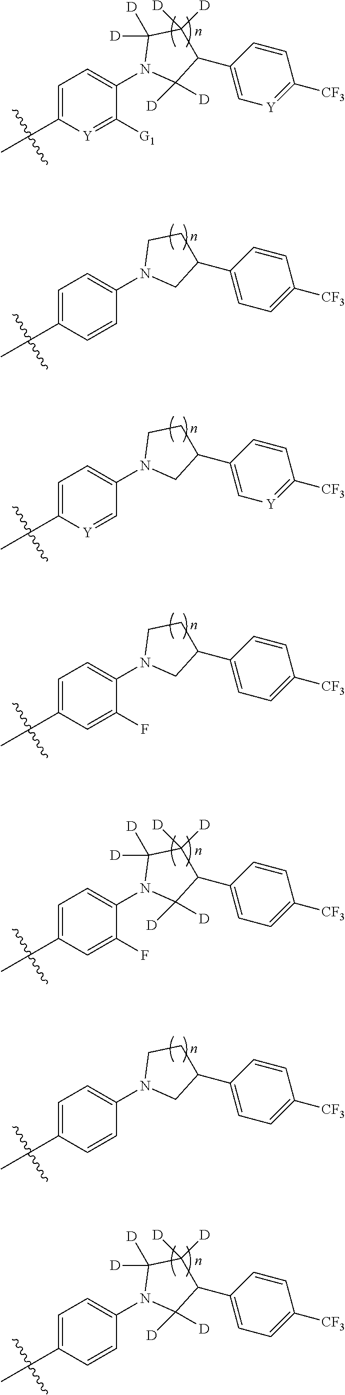

Or any of the following:

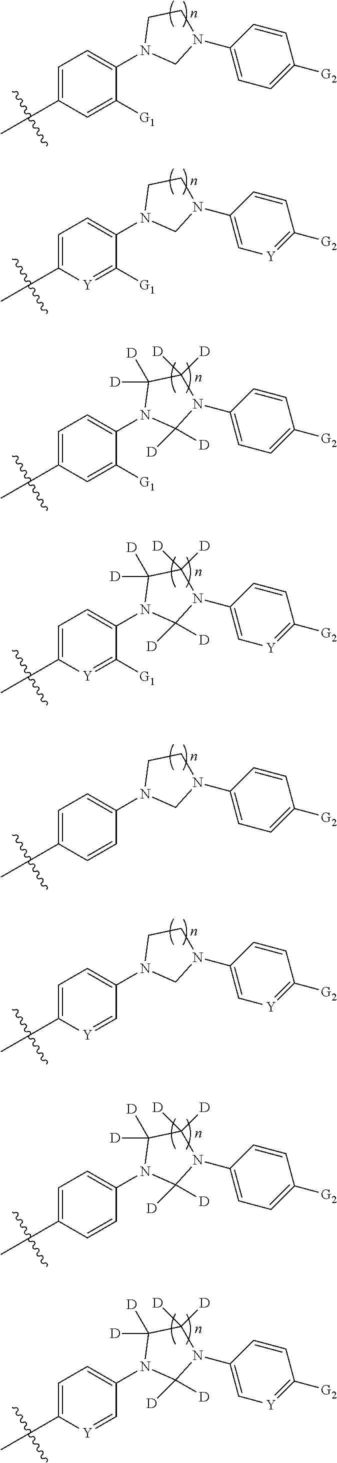

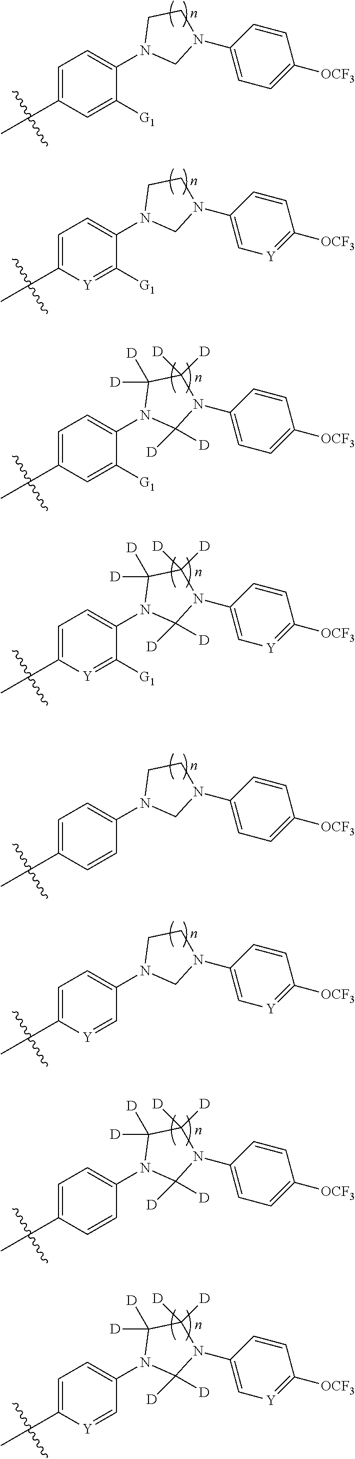

Or any of the following:

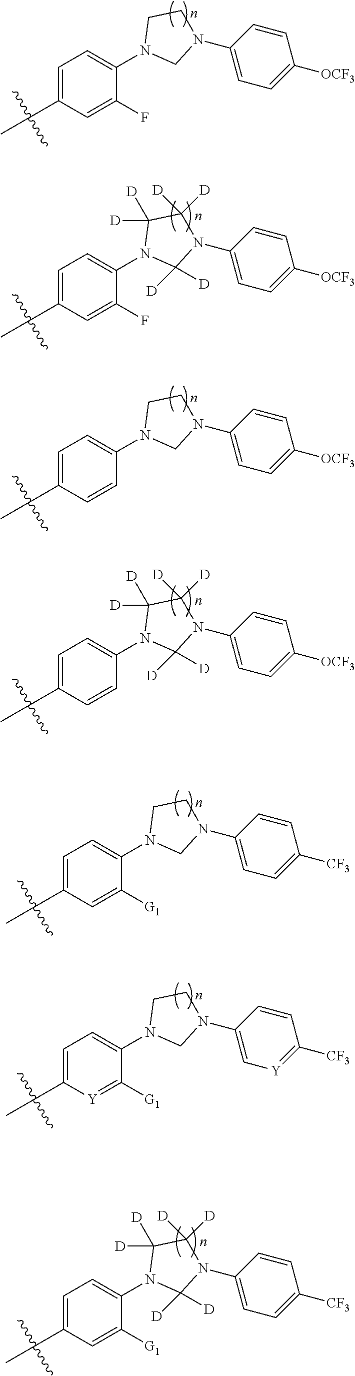

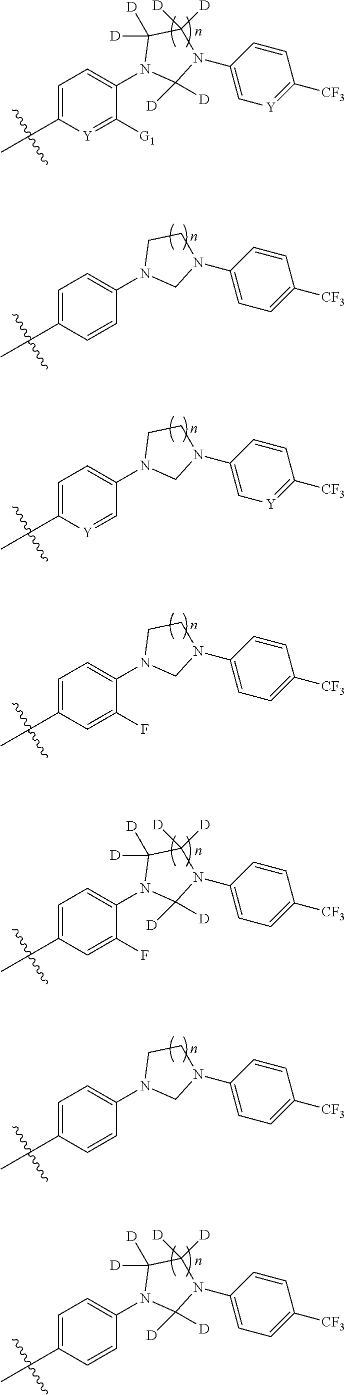

Or any of the following:

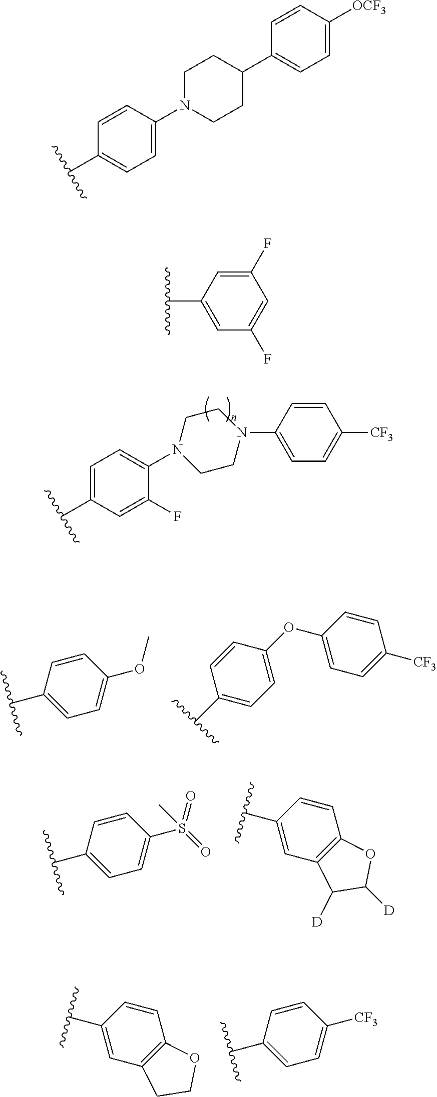

Or any of the following:

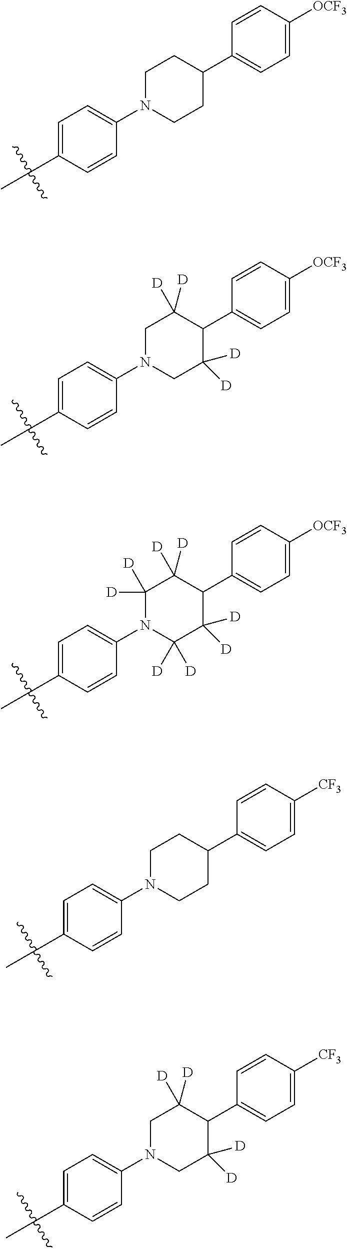

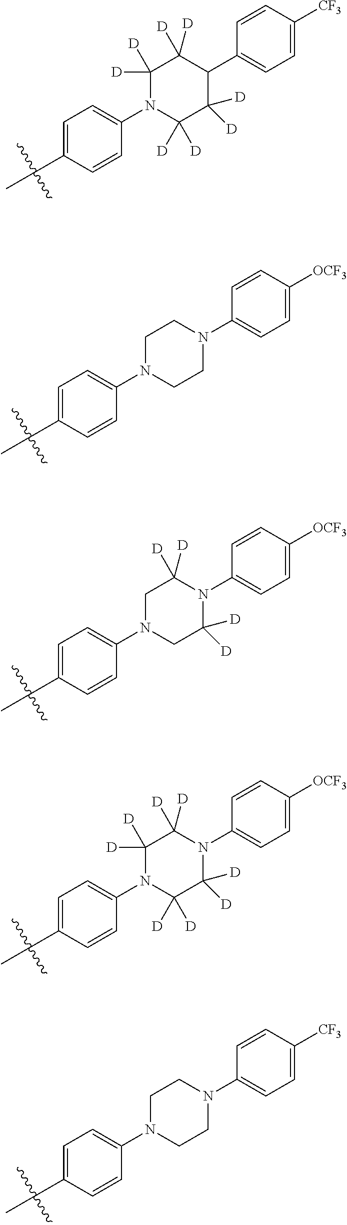

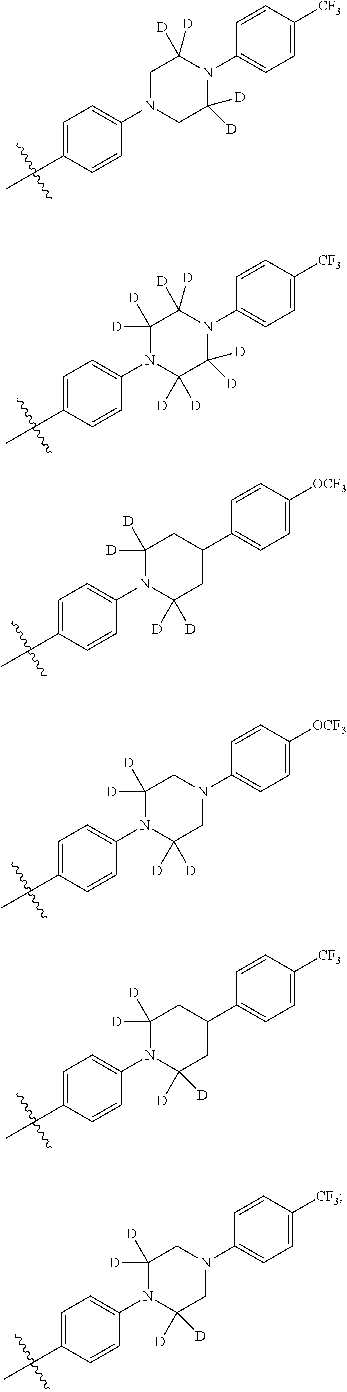

wherein, G₁ is H, D, CD₃, Cl, Br, CF₃, OCF₃, SF₅, SF₃, F, —P(O)(CH₃)₂, SO₂CH₃, alkyl, deuterated alkyl, halogen, heterocycle deuterated heterocycle, alkoxy, deuterated alkoxy, aryl, deuterated aryl, heteroaryl, deuterated heteroaryl, amine, substituted amine, deuterated amine, cycloalkyl, deuterated cycloalkyl and n is 0, 1, 2, 3, or 4; and wherein, G₂ is H, D, CD₃, Cl, Br, CF₃, OCF₃, SF₅, SF₃, F, —P(O)(CH₃)₂, SO₂CH₃, alkyl, deuterated alkyl, halogen, heterocycle deuterated heterocycle, alkoxy, deuterated alkoxy, aryl, deuterated aryl, heteroaryl, deuterated heteroaryl, amine, substituted amine, deuterated amine, cycloalkyl, deuterated cycloalkyl and n is 0, 1, 2, 3, or 4;

or any of the R₃ groups given at (a)-(v), formulas (Ib)-(Iv) hereinabove;

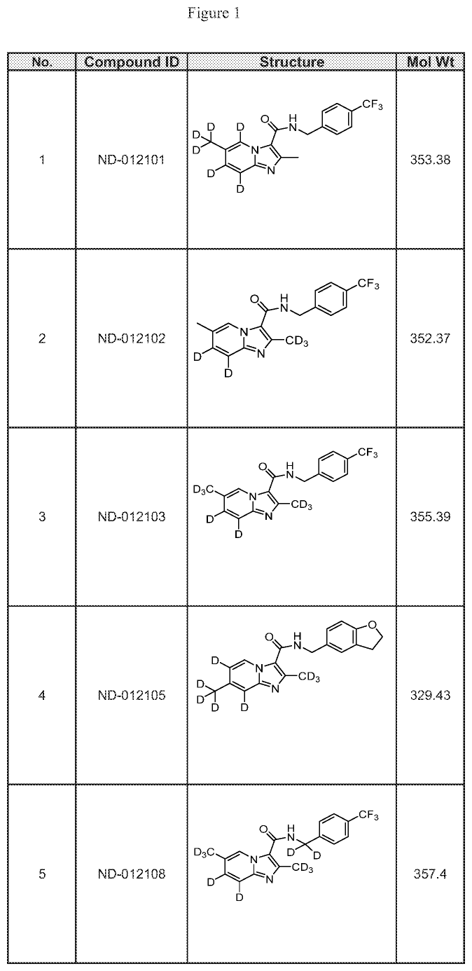

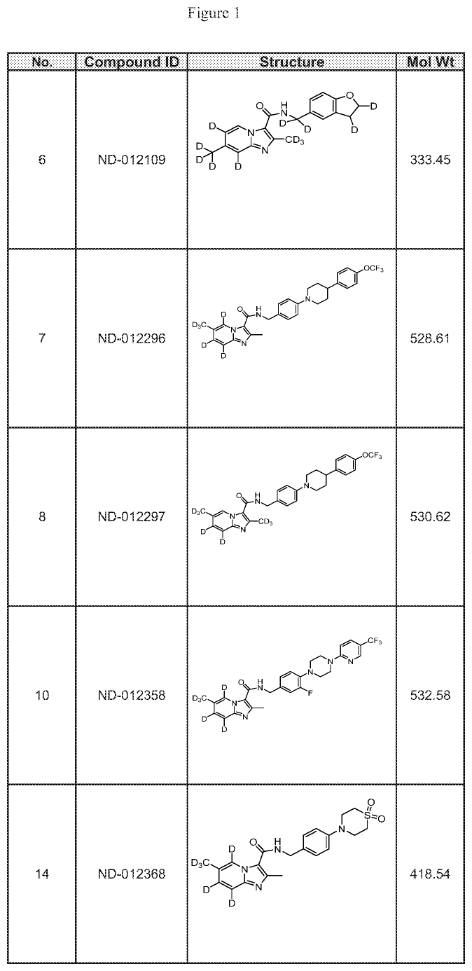

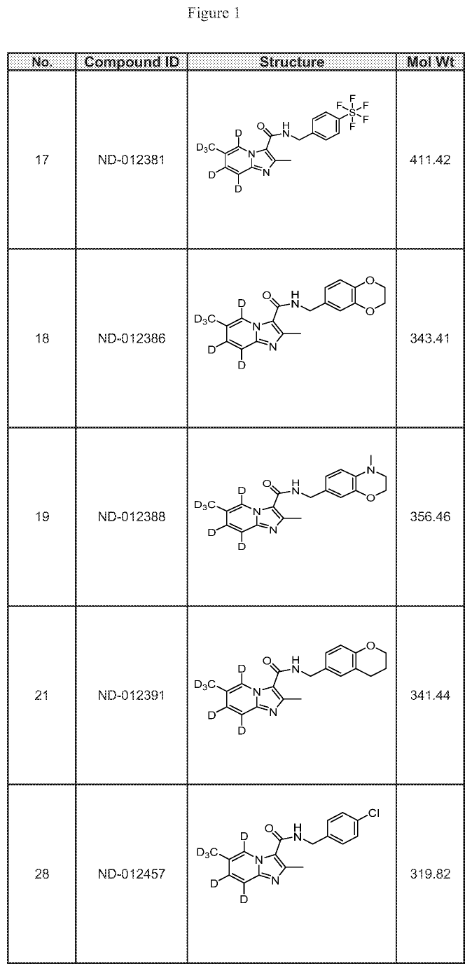

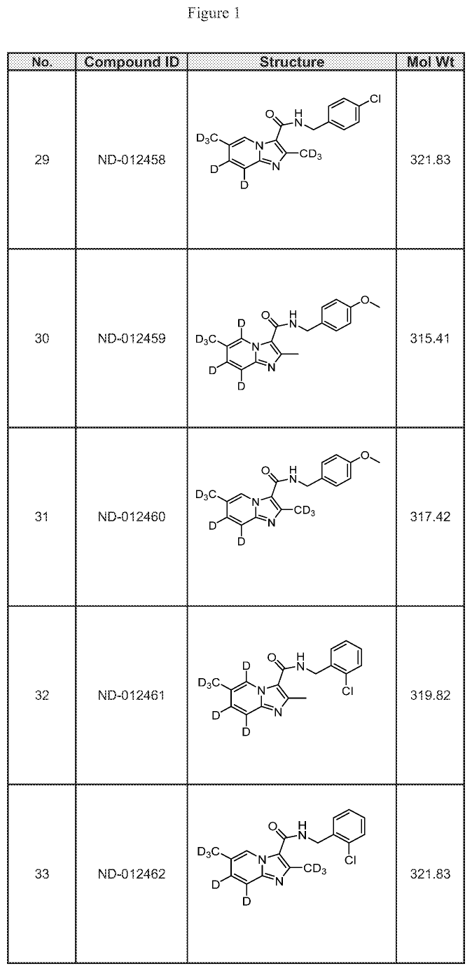

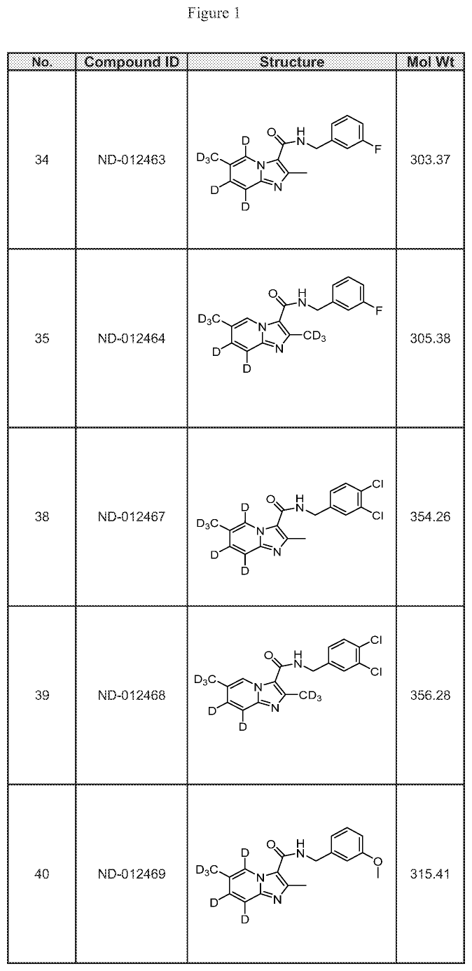

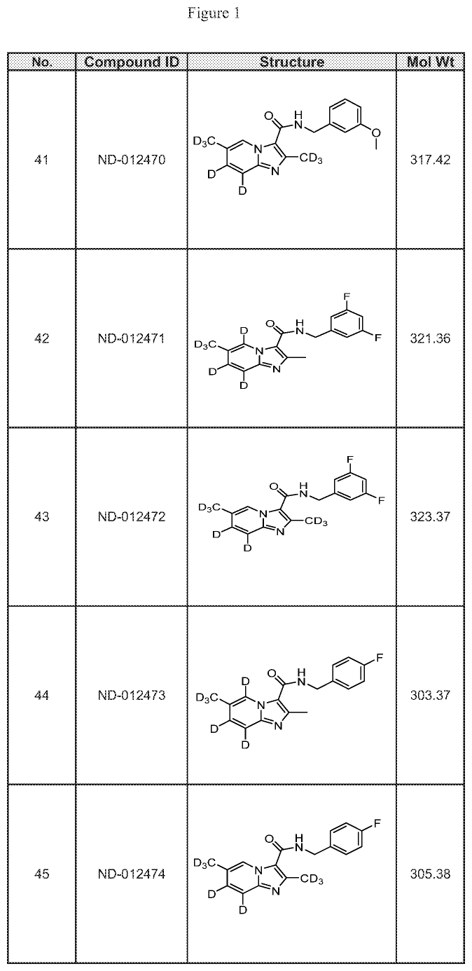

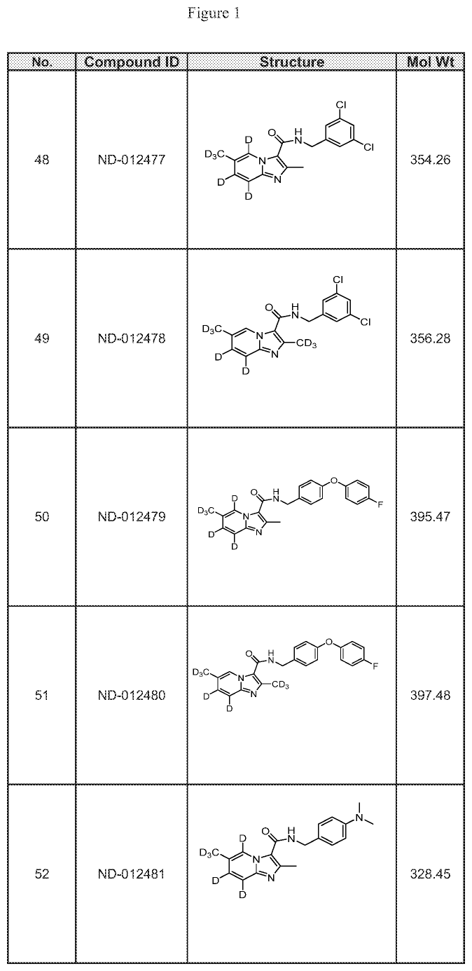

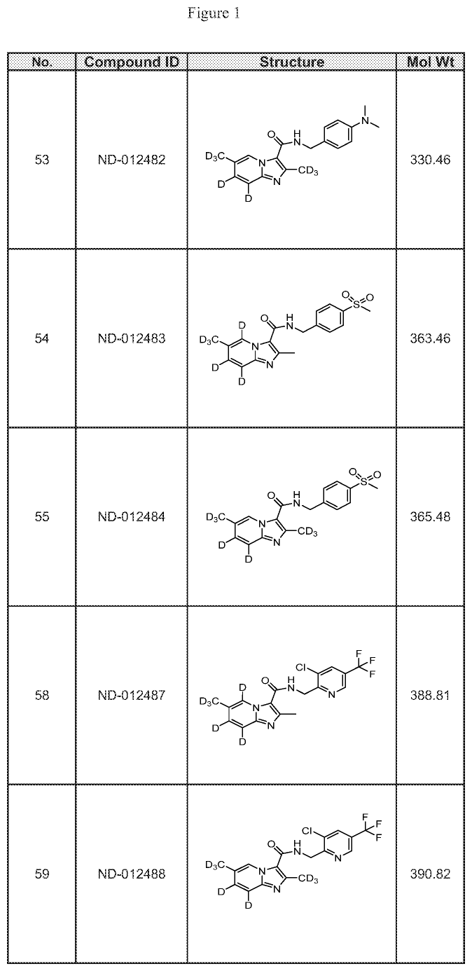

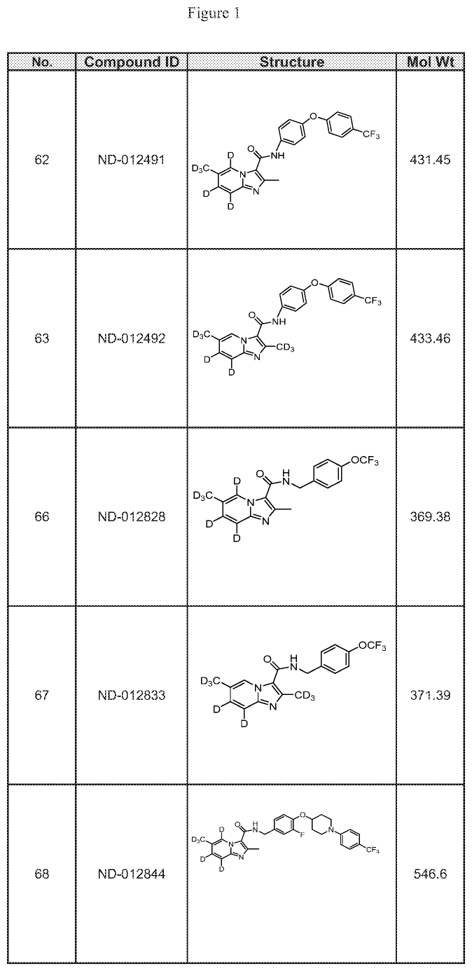

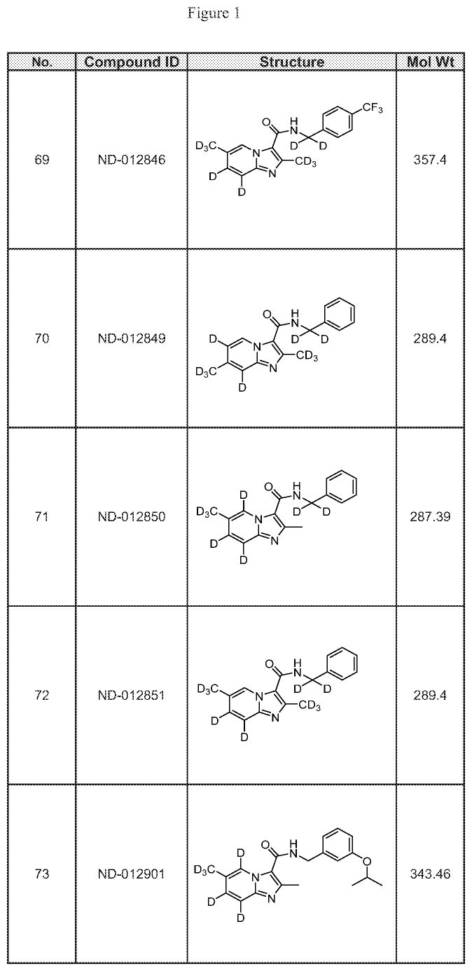

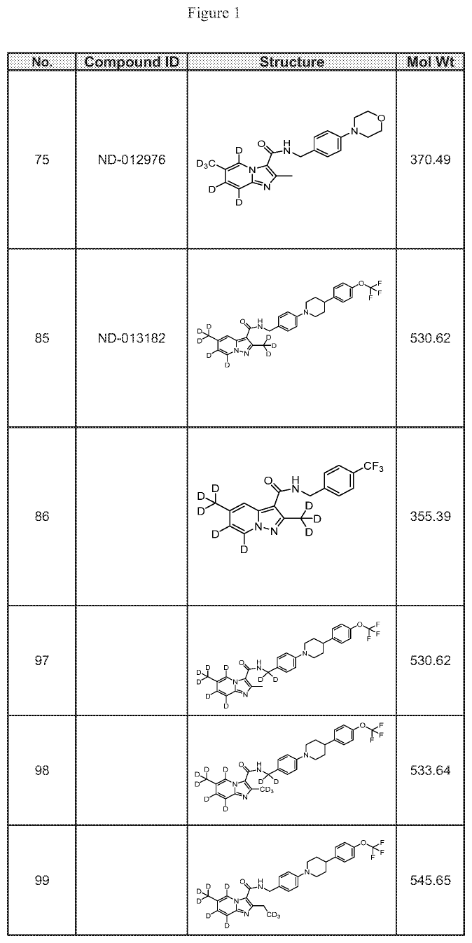

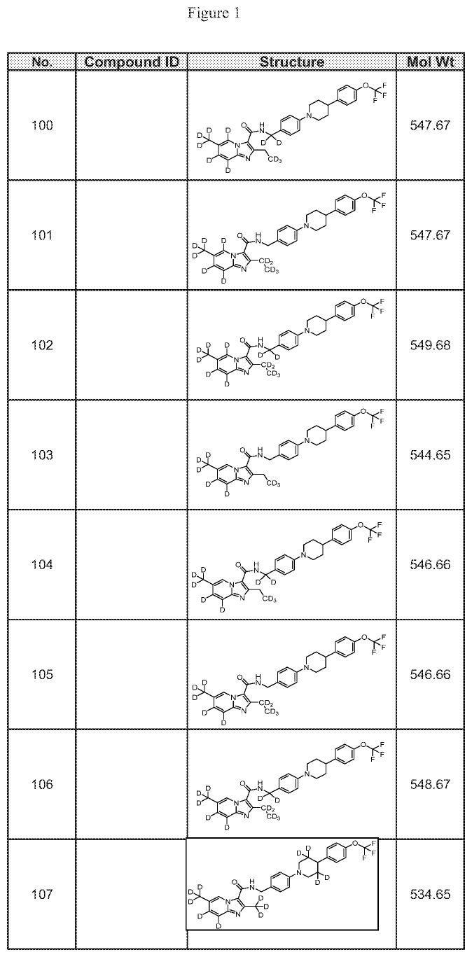

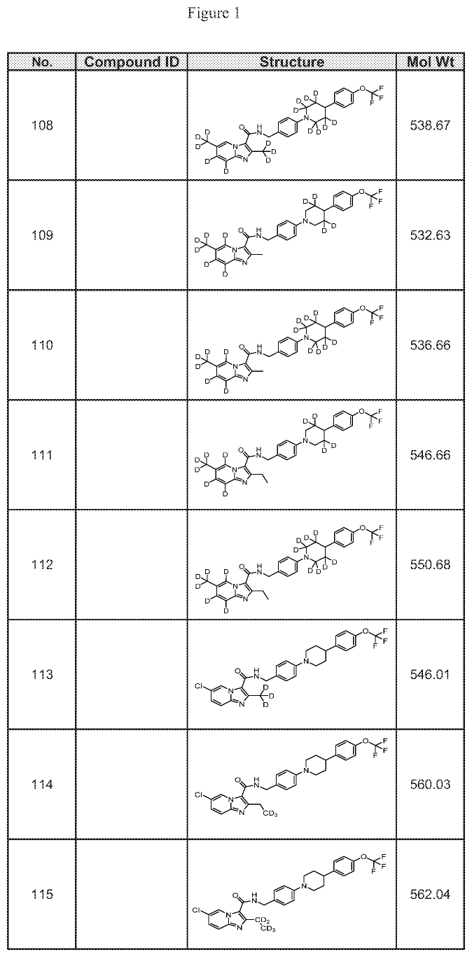

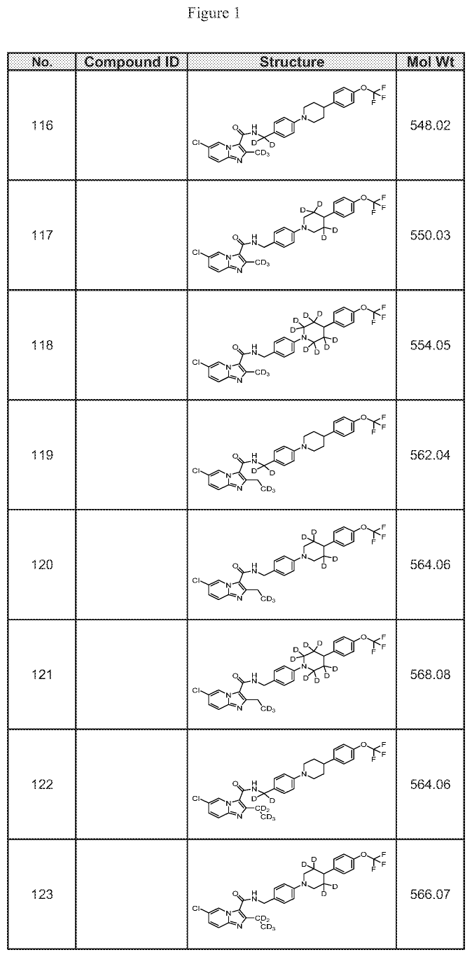

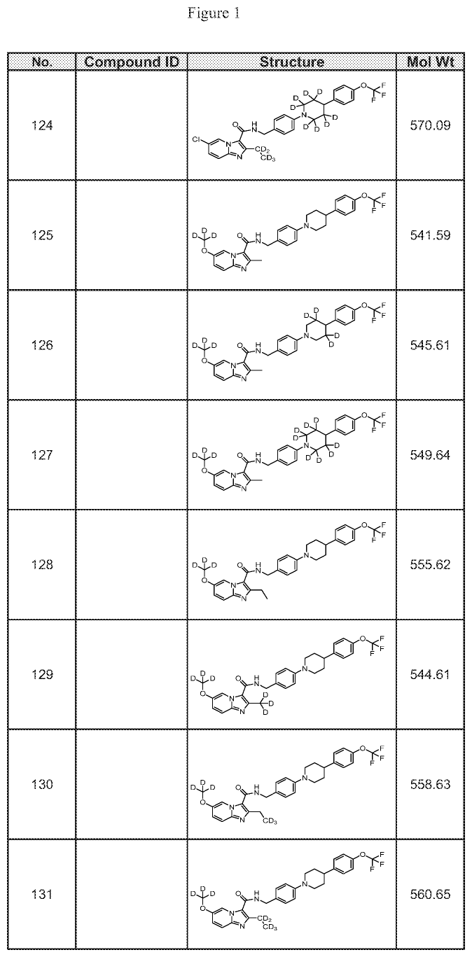

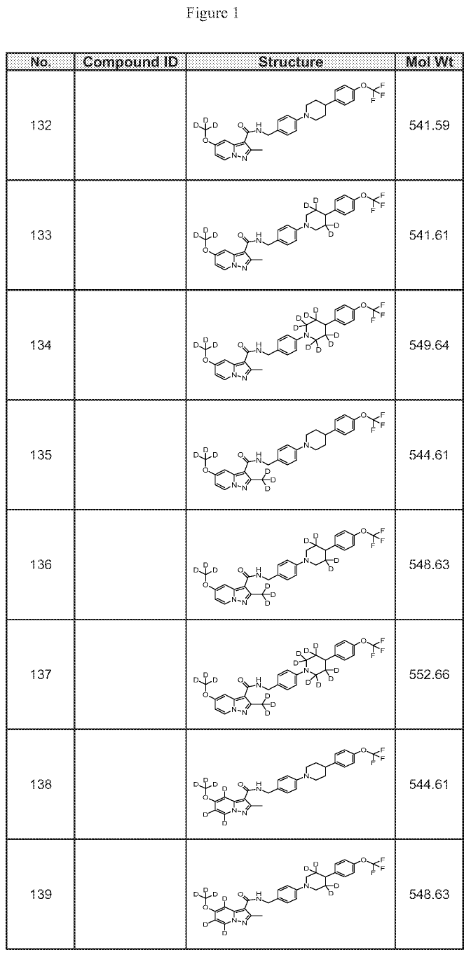

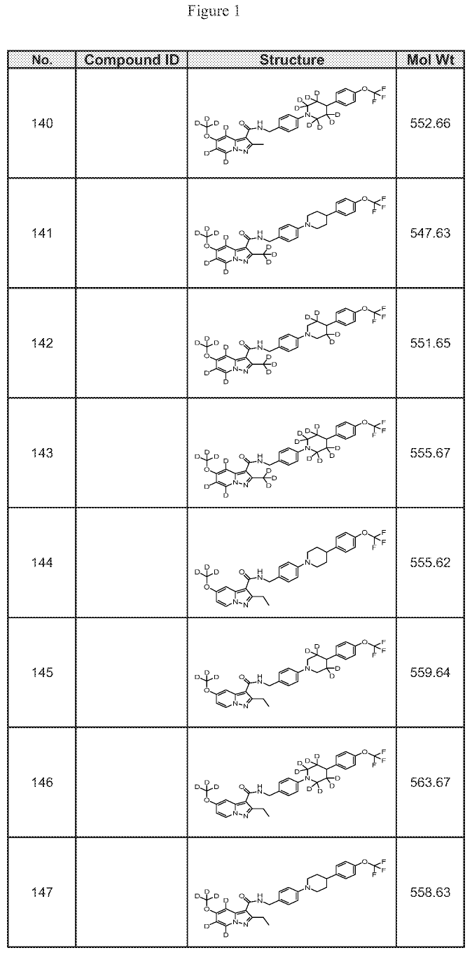

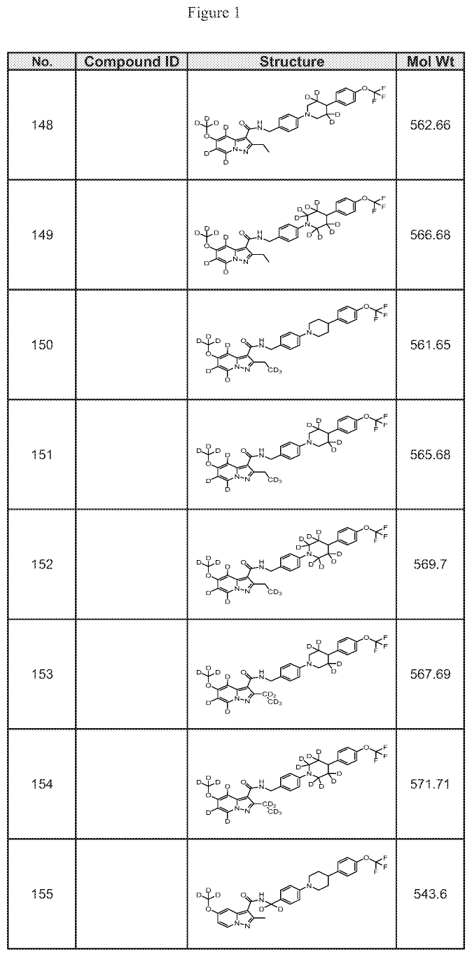

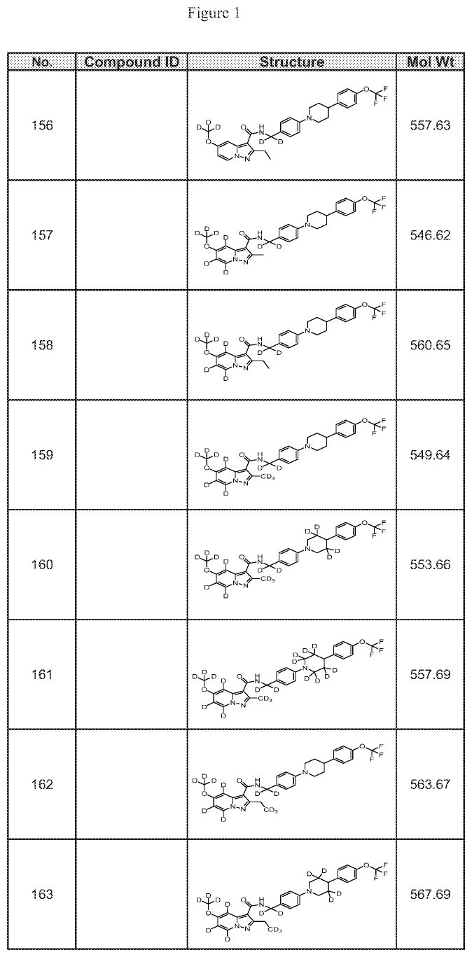

or any of the R₃ groups shown at the corresponding location in the compounds of FIG. 1;

or salt thereof.

It should be clear that the “n” subscripts in the ring groups above, are not the same “n” as those in the —(CH₂)ₙ— and —(CD₂)ₙ-groups that link the Z group to the R₃ group. But, in each case they may independently be 0, 1, 2, 3, or 4.

In some embodiments, G₁ is H, D, CD₃, Cl, Br, CF₃, OCF₃, SF₅, SF₃, F, —P(O)(CH₃)₂, SO₂CH₃, alkyl, deuterated alkyl, or halogen.

In some embodiments, G₂ is H, D, CD₃, Cl, Br, CF₃, OCF₃, SF₅, SF₃, F, —P(O)(CH₃)₂, SO₂CH₃, alkyl, deuterated alkyl, or halogen.

Other embodiments of R₃ include one of the following formulas:

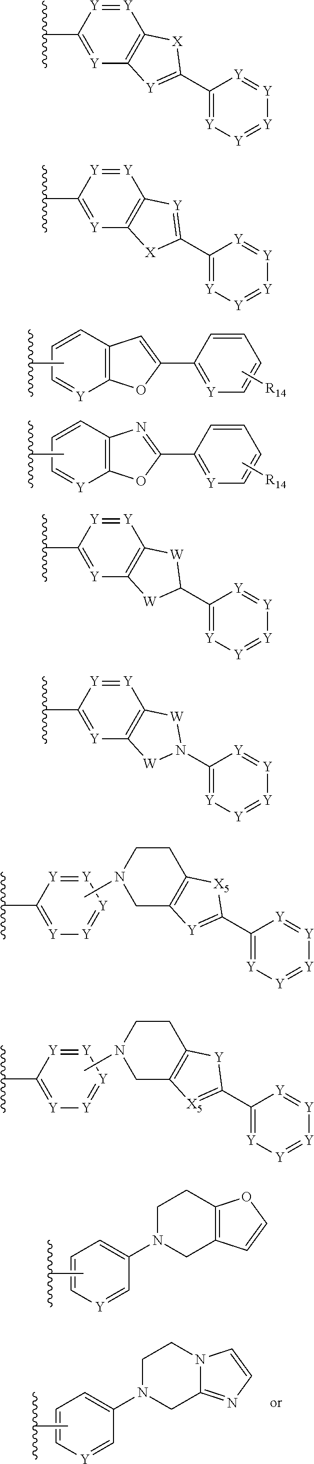

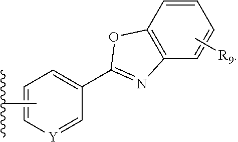

As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X or the same or different pharmaceutically acceptable salts thereof. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is disubstituted.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

As is generally known, and as used herein, the term, “deuterated” means the replacement of one or more carbon-hydrogen bonds with one or more isotopic carbon-deuterium bond.

For example, “deuterated alkyl” means an alkyl in which at least one or more carbon hydrogen bonds are replaced with isotopic carbon-deuterium bonds.

The term “halogen” or “halo” refers to fluoro, bromo, chloro, or iodo substituents.

The term “alkyl” refers to a cyclic, branched, or straight chain alkyl group containing carbon and hydrogen, and unless otherwise mentioned contains one to twelve carbon atoms. This term may be further exemplified by groups such as methyl, ethyl, n-propyl, isopropyl, isobutyl, t-butyl, pentyl, pivalyl, heptyl, adamantyl, and cyclopentyl. Alkyl groups can either be unsubstituted or substituted with one or more substituents, for instance, —P(O)(CH₃)₂, SO₂CH₃, halogen, alkyl, alkoxy, alkylthio, trifluoromethyl, acyloxy, hydroxy, mercapto, carboxy, aryloxy, aryl, arylalkyl, heteroaryl, amino, alkylamino, dialkylamino, morpholino, piperidino, pyrrolidin-1-yl, piperazin-1-yl, or other functionality to form a “substituted alkyl” or “functionalized alkyl”. An alkyl may be an extended alkyl.

The term, “extended alkyl” refers to —CH₂—(CH₂)ₙ—CH₃, wherein n is an integer of 2 to 8, or —CH₂—(CH₂)ₙ-alkyl, wherein n is an integer of 1 to 8, or —CH₂—(CH₂)ₙ-cycloalkyl, wherein n is an integer of 1 to 8. The extended alkyl, or alkyl group thereof, or both, may be branched or unbranched. The extended alkyl or alkyl group thereof, or both, can either be unsubstituted or substituted with one or more substituents, for instance, —P(O)(CH₃)₂, SO₂CH₃, halogen, alkyl, alkoxy, alkylthio, trifluoromethyl, acyloxy, hydroxy, mercapto, carboxy, aryloxy, aryl, arylalkyl, heteroaryl, amino, alkylamino, dialkylamino, morpholino, piperidino, pyrrolidin-1-yl, piperazin-1-yl, or other functionality to form a “substituted extended alkyl” or “functionalized extended alkyl”. Examples include —CH₂—(CH₂)ₙ-cyclopropyl, wherein n is an integer of 1 to 8; —CH₂—(CH₂)ₙ-butyl or -t-butyl, wherein n is an integer of 0 to 8, and the like.

When an alkyl group can be substituted, it can thus be a “substituted alkyl”. The term “substituted alkyl” refers to an alkyl moiety that can include 1-4 substituents selected from halogen, het, cycloalkyl, cycloalkenyl, aryl, amino, cyano, nitro, —P(O)(CH₃)₂, SO₂CH₃, —OQ₁₀, —SQ₁₀, —S(O)₂Q₁₀, —S(O)₂Q10, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —N(Q₁₀)C(S)NQ₁₀Q₁₀, —C(O)NQ₁₀Q₁₀, —C(S)NQ₁₀Q₁₀, —C(O)C(Q₁₆)₂₀C(O)Q₁₀, —CN, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, and —SNQ₁₀Q₁₀. Each of the het, cycloalkyl, cycloalkenyl, and aryl can be optionally substituted with 1-4 substituents independently selected from halogen and Q₁₅.

The term “alkoxy” refers to a group having the general formula —OR, wherein R is an alkyl group. The alkyl group, for example, may be a cyclic, branched, or straight chain alkyl group containing carbon and hydrogen, and unless otherwise mentioned contains one to twelve carbon atoms, which may be substituted, or unsubstituted.

The term “deuterated alkoxy” means an alkoxy in which at least one or more carbon hydrogen bonds are replaced with isotopic carbon-deuterium bonds.

When an alkoxy group can be substituted, it can thus be a “substituted alkoxy”. The term “substituted alkoxy” refers to an alkoxy moiety that can include 1-4 substituents selected from halogen, het, cycloalkyl, cycloalkenyl, aryl, amino, cyano, nitro, —P(O)(CH₃)₂, SO₂CH₃, —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —N(Q₁₀)C(S)NQ₁₀Q₁₀, —C(O)NQ₁₀Q₁₀, —C(S)NQ₁₀Q₁₀, —C(O)C(Q₁₆)₂₀C(O)Q₁₀, —CN, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, and —SNQ₁₀Q₁₀. Each of the het, cycloalkyl, cycloalkenyl, and aryl can be optionally substituted with 1-4 substituents independently selected from halogen and Q₁₅.

The term “cycloalkyl” refers to a cyclic alkyl moiety. Unless otherwise stated, cycloalkyl moieties include between 3 and 8 carbon atoms, which may be substituted or unsubstituted, functionalized or not functionalized, not extended, or extended.

The term “deuterated cycloalkyl” means a cycloalkyl in which at least one or more carbon hydrogen bonds are replaced with isotopic carbon-deuterium bonds.

The term “alkene” refers to a hydrocarbon molecule with the general formula CₙH₂ₙ that contains one or more double bonds.

The term “alkyne” refers to a moiety having the general formula C₂H₂ₙ₋₂ corresponding to carbon chains with a triple carbon-carbon bond included.

The term “alcohol” refers to any organic compound in which a hydroxyl group (—OH) is bound to a carbon atom of an alkyl or substituted alkyl group. The general formula for simple acyclic alcohols is CₙH₂ₙ₊₁OH.

The term “epoxide” refers to any of a class of organic compound, cyclic ethers, having a three-member ring.

The term “ketone” refers to an organic compound containing the carbonyl group, >C═O, to which other carbon atoms are attached.

The term “ester” refers to the product of the reaction between a carboxylic acid and an alcohol.

The term “ether” refers to an organic compound containing the functional group RO—R′ where R and R′ are the organic groups such as alkyl or aryl.

The term “aldehyde” refers to an organic compound containing a —CHO group.

The term “nitrile” refers to any of a class of organic compounds containing the cyano radical —CN.

The term “thiol” refers to a molecular group that includes a bonded sulfur and hydrogen atom (—SH).

The term “thioester” refers to a compound resulting from the bonding of sulfur with an acyl group with the general formula R—S—CO—R′. Thioesters are the product of esterification between a carboxylic acid and a thiol (as opposed to an alcohol in regular esters).

The term “sulfide” refers to an organic compound containing sulfur bonded to carbon. The term “disulfide” refers to the structural unit composed of a linked pair of sulfur atoms.

The term “sulfone” refers to a chemical compound containing a sulfonyl functional group attached to two carbon atoms. The central sulfur atom is twice double bonded to oxygen and has two further hydrocarbon substituents. The general structural formula is R—S(═O)₂R′ where R and R′ are the organic groups such as alkyl or aryl, or a portion of a formula described herein. For example, a methylsulfone group is a —S(═O)₂Me group.

The term “sulfoxide” refers to a chemical compound containing a sulfonyl functional group attached to two carbon atoms. Sulfoxides can be considered oxidized sulfides.

The term “amine” refers to NH₂, NHR, or NR₂. Unless otherwise stated R can be alkyl, alkenyl, alkynyl, cycloalkyl, cycloalkenyl, het or aryl.

The term “deuterated amine” means an amine in which at least one or more carbon hydrogen bonds are replaced with isotopic carbon-deuterium bonds.

The term “alkyl amino” refers to a group in which an alkyl or alkyl groups are attached to the remainder of a molecule via nitrogen.

The term “deuterated alkyl amino” means an alkyl amino in which at least one or more carbon hydrogen bonds are replaced with isotopic carbon-deuterium bonds.

The term “amide” refers to an organic compound containing the —CONH— group.

The term “urea” refers to an organic compound with the chemical formula (NH₂)₂CO or RNHCONHR′ where R and R′ are the organic groups such as alkyl or aryl, or a portion of a formula described herein.

The term “carbamate” refers to any of a group of organic compounds sharing a common functional group with the general structure —NH(CO)O—. Carbamates are esters of carbamic acid, NH₂COOH. Since carbamic acid contains nitrogen attached to a carboxyl group, it is also an amide. Therefore, carbamate esters may have alkyl or aryl groups substituted on the nitrogen, or the amide function. For example, ethyl carbamate is unsubstituted, whereas ethyl N-methylcarbamate has a methyl group attached to the nitrogen.

The term “nitro” refers to NO₂.

The term “aryl” refers to phenyl, substituted phenyl, naphthyl, and substituted naphthyl.

The term “deuterated aryl” means an aryl in which at least one or more carbon hydrogen bonds are replaced with isotopic carbon-deuterium bonds.

Aryl groups can either be unsubstituted or substituted with one or more substituents. When an aryl group can be substituted, it can thus be a “substituted aryl”.

The term “substituted aryl” refers to an aryl moiety having 1-3 substituents selected from halogen, het, alkyl, substituted alkyl, alkenyl, alkynyl, alkoxy, substituted alkoxy, cycloalkyl, cycloalkenyl, aryl, cyano, nitro, —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂OC(O)Q₁₀, —NQ₁₀C(O)Q₁₀, —N(Q₁₀)C(S)NQ₁₀Q₁₀, —N(Q₁₀)C(S)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —SNQ₁₀Q₁₀, —P(O)(CH₃)₂, SO₂CH₃, and SF₅. The het, cycloalkyl, cycloalkenyl, alkenyl, alkynyl, and aryl being optionally substituted with 1-3 substituents selected from halogen and Q₁₅.

The term “aryloxy” refers to a group having the general formula —OR, wherein R is an aryl group. The aryl group, may be, for example, substituted aryl, phenyl, substituted phenyl, naphthyl, and substituted naphthyl.

The term “deuterated aryloxy” means an aryloxy in which at least one or more carbon hydrogen bonds are replaced with isotopic carbon-deuterium bonds.

The term “morpholine” refers to the cyclic organic compound or moiety having the chemical formula O(CH₂CH₂)₂NH. This heterocycle features both amine and ether functional groups. Because of the amine, morpholine is a base; its conjugate acid is called morpholinium. For example, when morpholine is neutralized by hydrochloric acid, one obtains the salt morpholinium chloride. Morpholine can be a substituent of organic groups such as alkyl and aryl.

The term “thiomorpholine” refers to C₄H₉NS, and is a heterocyclic compound containing nitrogen and sulfur. It may be considered a thio derivative of morpholine.

The term “piperazine” refers to an organic compound that consists of a six-member ring containing two opposing nitrogen atoms.

The term “piperidine” refers to an organic compound with the molecular formula (CH₂)₅NH. This heterocyclic amine consists of a six-member ring containing five methylene units and one nitrogen atom.

The term “acyl” refers to any of a group or radical of the form RCO— where R is an organic group such as alkyl or aryl.

The term “furan” refers to any of a class of aromatic heterocyclic compounds containing a ring of four carbon atoms and an oxygen atom; for instance, C₄H₄₀. The term “nitrofuran” refers to a furan ring with a nitro group substituent.

The term “thiophene” refers to a heterocyclic compound with the formula C₄H₄S. Consisting of a flat five-membered ring, it is aromatic as indicated by its extensive substitution reactions. Related to thiophene are benzothiophene and dibenzothiophene, containing the thiophene ring fused with one and two benzene rings, respectively. The term “nitrothiophene” refers to a thiophene ring with a nitro group substituent. Compounds analogous to thiophene include furan (C₄H₄O) and pyrrole (C₄H₄NH).

The term “imidazole” refers to an organic compound with the formula C₃H₄N₂. This aromatic heterocycle is classified as an alkaloid. Imidazole refers to the parent compound whereas imidazoles are a class of heterocycles with similar ring structure but varying substituents. A nitroimidazole is an imidazole derivative that contains a nitro group.

The term “oxazole” refers to a five-member heterocycle having three carbon atoms, one oxygen atom, one nitrogen atom and two double bonds; the 1,3-isomer is aromatic.

The term “oxazoline” refers to an unsaturated heterocyclic compound containing a five-member ring, two double bonds, one nitrogen and one oxygen atom; and any derivative of this compound.

The term “thiazole” refers to any of a class of unsaturated heterocyclic compounds containing a ring of three carbon atoms, a sulfur and an nitrogen atom; for instance the simplest one, C₃H₃SN.

The term “thiazoline” refers to an unsaturated heterocyclic compound containing a five-member ring, two double bonds, one nitrogen and one sulfur atom; and any derivative of this compound.

The term “triazole” refers to either one of a pair of isomeric chemical compounds with molecular formula C₂H₃N₃, having a five-member ring of two carbon atoms and three nitrogen atoms.

The term “pyridine” refers to any of a class of aromatic heterocyclic compounds containing a ring of five carbon atoms and a nitrogen atom; for instance, the simplest one, C₅H₅N.

The term “pyrazine” refers to a diazine in which the two nitrogen atoms are in the para-position.

The term “naphthalene” refers to an aromatic, white, solid hydrocarbon with formula C₁₀H₈ and the structure of two fused benzene rings.

The term “diketopiperazine” refers to a class of cyclic organic compounds that result from peptide bonds between two amino acids to form a lactam. They are the smallest possible cyclic peptides.

The term “quinoline” refers to any of a class of aromatic heterocyclic compounds containing a benzene ring fused with a ring of five carbon atoms and a nitrogen atom; for instance the simplest one, C₉H₇N. Isoquinoline, also known as benzo[c]pyridine or 2-benzanine, is a heterocyclic aromatic organic compound. It is a structural isomer of quinoline. Isoquinoline and quinoline are benzopyridines, which are composed of a benzene ring fused to a pyridine ring. In a broader sense, the term isoquinoline is used to make reference to isoquinoline derivatives.

The term “oxazolidinone” refers to a class of heterocyclic organic compounds containing both nitrogen and oxygen in a 5-member ring.

The terms “heterocyclic,” “heterocycle,” and “het” are used interchangeably and refer to organic compounds containing at least one atom of carbon, and at least one element other than carbon, such as sulfur, oxygen or nitrogen within a ring structure. These structures may comprise either simple aromatic rings or non-aromatic rings. Each monocyclic ring may be aromatic, saturated or partially unsaturated. A bicyclic ring system may include a mono-cyclic ring containing one or more heteroatom fused with a cycloalkyl or aryl group. A bicyclic ring system may also include a monocyclic ring containing one or more heteroatom fused with another monocyclic ring system.

The term “deuterated heterocycle” means a heterocycle in which at least one or more carbon hydrogen bonds are replaced with isotopic carbon-deuterium bonds.

Examples of “heterocycles” include but are not limited to pyridine, thiophene, furan, pyrazoline, pyrimidine, 2-pyridyl, 3-pyridyl, 4-pyridyl, 2-pyrimidinyl, 4-pyrimidinyl, 5-pyrimidinyl, 3-pyrazinyl, 3-pyridazinyl, 4-pyridazinyl, 4-oxo-2-imidazolyl, 1,2,4-oxadiazole, 1,3,4-oxadiazole, 4-pyridazinyl, 3-pyrazinyl, 4-oxo-2-imidazolyl, 2-imidazolyl, 4-imidazolyl, 3-isoxazolyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,5-oxadiazole, 2-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl, 5-isothiazolyl, 1,3,4-oxadiazole, 4-oxo-2-thiazolinyl, 5-methyl-1,3,4-thiadiazol-2-yl, thiazoledione, 1,2,3,4-thiatriazole, 1,2,4-dithiazolone, phthalimide, quinolinyl, morpholinyl, benzimidazolyl, benzo[d]thiazolyl, benzo[d]oxazolyl, diazinyl, triazinyl, quinolinyl, quinoxalinyl, naphthyridinyl, azetidinyl, pyrrolidinyl, hydantoinyl, oxathiolanyl, dioxolanyl, imidazolidinyl, azabicyclo [2.2.1] heptyl, 2-methyl-1,4-dioxa-8-azaspiro[4.5]decane, 2,3-dimethyl-1,4-dioxa-8-azaspiro[4.5]decane, 3-methyl-1,5-dioxa-9-azaspiro[5.5]undecane, and 2,4-dimethyl-1,5-dioxa-9-azaspiro[5.5]undecane.

Heterocycle groups can either be unsubstituted or substituted with one or more substituents. When a heterocycle group can be substituted, it can thus be a “substituted heterocycle”.

The term “substituted heterocycle” refers to a heterocycle moiety having 1-3 substituents selected from halogen, het, alkyl, substituted alkyl, alkenyl, alkynyl, alkoxy, substituted alkoxy, cycloalkyl, cycloalkenyl, aryl, cyano, nitro, —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂₀C(O)Q₁₀, —NQ₁₀C(O)Q₁₀, —N(Q₁₀)C(S)NQ₁₀Q₁₀, —N(Q₁₀)C(S)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, and —SNQ₁₀Q₁₀, —P(O)(CH₃)₂. The het, cycloalkyl, cycloalkenyl, alkenyl, alkynyl and aryl being optionally substituted with 1-3 substituents selected from halogen and Q₁₅.

The term “heteroaryl” refers to a mono- or bicyclic het in which one or more cyclic ring is aromatic. Heteroaryl groups can either be unsubstituted or substituted with one or more substituents. When a heteroaryl group can be substituted, it can thus be a “substituted heteroaryl”.

The term “deuterated heteroaryl” means a heteroaryl in which at least one or more carbon hydrogen bonds are replaced with isotopic carbon-deuterium bonds.

The term “substituted heteroaryl” refers to a heteroaryl moiety substituted with one or more functional groups selected from halogen, alkyl, hydroxyl, amino, alkoxy, cyano, and nitro.

The term “substituted heteroaryl” refers to an heteroaryl moiety having 1-3 substituents selected from halogen, het, alkyl, substituted alkyl, alkenyl, alkynyl, alkoxy, substituted alkoxy, cycloalkyl, cycloalkenyl, aryl, cyano, nitro, —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₆)₂₀C(O)Q₁₀, —NQ₁₀C(O)Q₁₀, —N(Q₁₀)C(S)NQ₁₀Q₁₀, —N(Q₁₀)C(S)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, and —SNQ₁₀Q₁₀, —P(O)(CH₃)₂. The het, cycloalkyl, cycloalkenyl, alkenyl, alkynyl and aryl being optionally substituted with 1-3 substituents selected from halogen and Q₁₅.

Each Q₁₀ is independently selected from H, alkyl, cycloalkyl, het, cycloalkenyl, and aryl. The alkyl, het, cycloalkyl, cycloalkenyl, and aryl being optionally substituted with 1-3 substituents selected from halo, aryl optionally substituted with CF₃, and Q₁₃.

Each Q₁₁ is independently selected from H, halogen, alkyl, aryl, cycloalkyl, and het. The alkyl, aryl, cycloalkyl, and het being optionally substituted with 1-3 substituents independently selected from halogen, nitro, cyano, ═S, =0, and Q₁₄.

Each Q₁₃ is independently selected from Q₁₁, —OQ₁₁, —SQ₁₁, —S(O)₂Q₁₁, —S(O)Q₁₁, —OS(O)₂Q₁₁, —C(═NQ₁₁)Q₁₁, —S(O)₂—N═S(O)(Q)₂, —S(O)₂—N═S(Q)₂, —SC(O)Q₁₁, —NQ₁₁Q₁₁, —C(O)Q₁₁, —C(S)Q₁₁, —C(O)OQ₁₁, —OC(O)Q₁₁, —C(O)NQ₁₁Q₁₁, —(S)NQ₁₁Q₁₁, —C(O)C(Q₁₆)₂OC(O)Q₁₀, —CN, ═O, ═S, —NQ₁₁C(O)Q₁₁, —NQ₁₁C(S)Q₁₁, —NQ₁₁C(O)NQ₁₁Q₁₁, —NQ₁₁C(S)NQ₁₁Q₁₁, —S(Q)₂NQ₁₁Q₁₁, —NQ₁₁S(O)₂Q₁₁, —NQ₁₁S(O)Q₁₁, —NQ₁₁SQ₁₁, —NO₂, and —SNQ₁₁Q₁₁.

Each Q₁₄ is independently selected from H, alkyl, cycloalkyl, phenyl, or naphthyl, each optionally substituted with 1-4 substituents independently selected from F, Cl, Br, I, —OQ₁₆, —SQ₁₆, —S(O)₂Q₁₆, —S(O)Q₁₆, —OS(O)₂Q₁₆, —NQ₁₆Q₁₆, —C(O)Q₁₆, —C(S)Q₁₆, —C(O)OQ₁₆, —NO₂, —C(O)NQ₁₆Q₁₆, —C(S)NQ₁₆Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, and —NQ₁₆S(O)₂Q₁₆. The alkyl, cycloalkyl, and cycloalkenyl being further optionally substituted with ═O or ═S.

Each Q₁₅ is independently selected from H, oxo, alkyl, alkyloxy, cycloalkyl, heteroaryl, phenyl, or naphthyl, each optionally substituted with 1-4 substituents independently selected from alkyl optionally substituted with 1-3 halogens, F, Cl, Br, I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(S)NQ₁₆Q₁₆, —C(O)C(Q₁₆)₂₀C(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl can be further optionally substituted with ═O or ═S.

Each Q₁₆ is independently selected from H, alkyl, and cycloalkyl. The alkyl and cycloalkyl optionally including 1-3 halogens.

In some embodiments, a pharmaceutical composition may be provided employing conventional techniques in unit dosage form containing effective or appropriate amounts of one or more active component. In various embodiments, the quantity of active component (compound) in a pharmaceutical composition and unit dosage form thereof may be varied or adjusted widely depending upon the particular application, the potency of the particular compound and the desired concentration. In an exemplary embodiment, the quantity of active component may range from 0.5% to 90% by weight of the composition.

In various embodiments, in therapeutic uses for treating, ameliorating, preventing, or combating a mycobacterial infection in a subject, such as an infection caused by M. tuberculosis or M. avium, the compounds or pharmaceutical compositions thereof may be administered orally, parenterally, and/or by inhalation at a dosage to obtain and maintain a concentration or blood-level of active component in the animal undergoing treatment that is therapeutically effective. In an embodiment, such a therapeutically effective amount/dosage of active component may be in the range of about 0.1 to about 300 mg/kg, or about 0.1 to about 100 mg/kg, for instance, about 0.1 to about 50 mg/kg, or about 0.1 to about 10 mg/kg, of body weight/day. It is to be understood that the dosages may vary depending upon the requirements of the patient, the severity of the infection, the particular mycobacterial species, whether the infection is latent or active, the drug resistance of the strain, the duration of the infection being treated, and the particular compound being used. Also, it is to be understood that the initial dosage administered may be increased beyond the above upper level in order to rapidly achieve the desired blood-level or the initial dosage may be smaller than the optimum and the daily dosage may be progressively increased during the course of treatment depending on the particular situation. If desired, the daily dose also may be divided into multiple doses for administration, for instance, two to four times per day.

### Pharmaceutical Formulations

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes. The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% (weight or mol %) of active compound. The percentage of the compositions and preparations can vary and may conveniently be from about 0.5% to about 60%, about 1% to about 25%, or about 2% to about 10%, of the weight of a given unit dosage form. This range includes all values and subranges in between, including 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, and 60%, or any combination thereof. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), U.S. Pat. No. 4,820,508 (Wortzman), U.S. Pat. No. 4,608,392 (Jacquet et al.), and U.S. Pat. No. 4,559,157 (Smith et al.). Such dermatological compositions can be used in combinations with the compounds described herein where an ingredient of such compositions can optionally be replaced by a compound described herein, or a compound described herein can be added to the composition

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

The compound can be conveniently administered in a unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The compounds described herein can be effective antimicrobial agents, for example, against various microbes that cause TB. The invention provides therapeutic methods of treating bacterial and/or TB infections in a mammal, which involve administering to a mammal having an infection an effective amount of a compound or composition described herein. A mammal includes a primate, human, rodent, canine, feline, bovine, ovine, equine, swine, caprine, bovine and the like.

The ability of a compound of the invention to kill a microbe or bacteria, to inhibit its growth, and/or to treat a related infection may be determined by using assays well known to the art. For example, the design of treatment protocols, toxicity evaluation, data analysis, quantification of cell kill, and the biological significance of the use of various screens are known. In addition, ability of a compound to treat an infection may be determined using the Tests described below.

### EXAMPLES

Deuterated imidazopyridine and pyrazolopyridine compounds were synthesized and tested as exemplary members of the new deuterated class of anti-mycobacterial agents disclosed herein. See, for example, the compounds set out in FIG. 1. The deuterated imidazopyridine and pyrazolopyridine class of molecules is unrepresented within the TB and M. avium literature, and the compounds are very attractive because of their ease of synthesis from C—H containing precursors, as shown, for example, in schemes 1, 4, 6 and 8 and from imidazopyridine and pyrazolopyridines themselves (scheme 2, 3 and 7, and 15-16 and 19), using a simple apparatus.

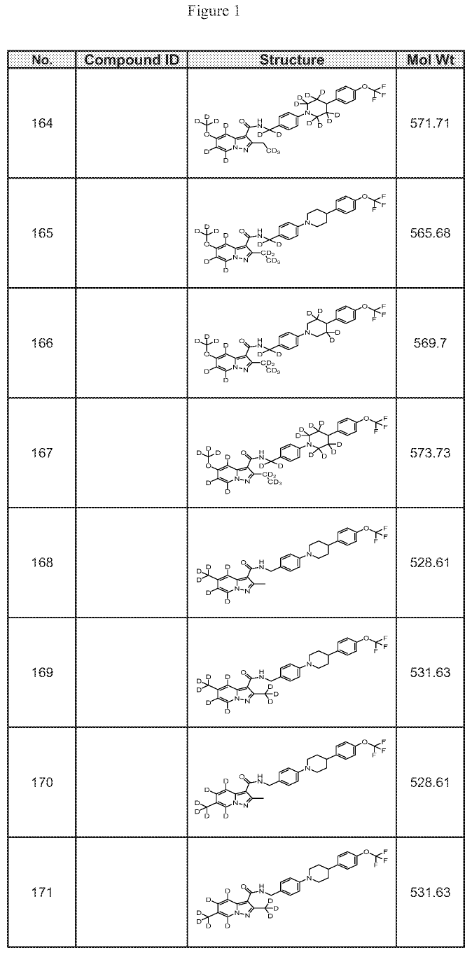

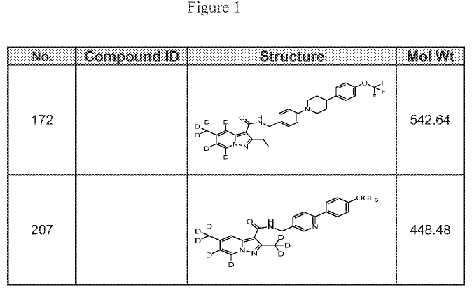

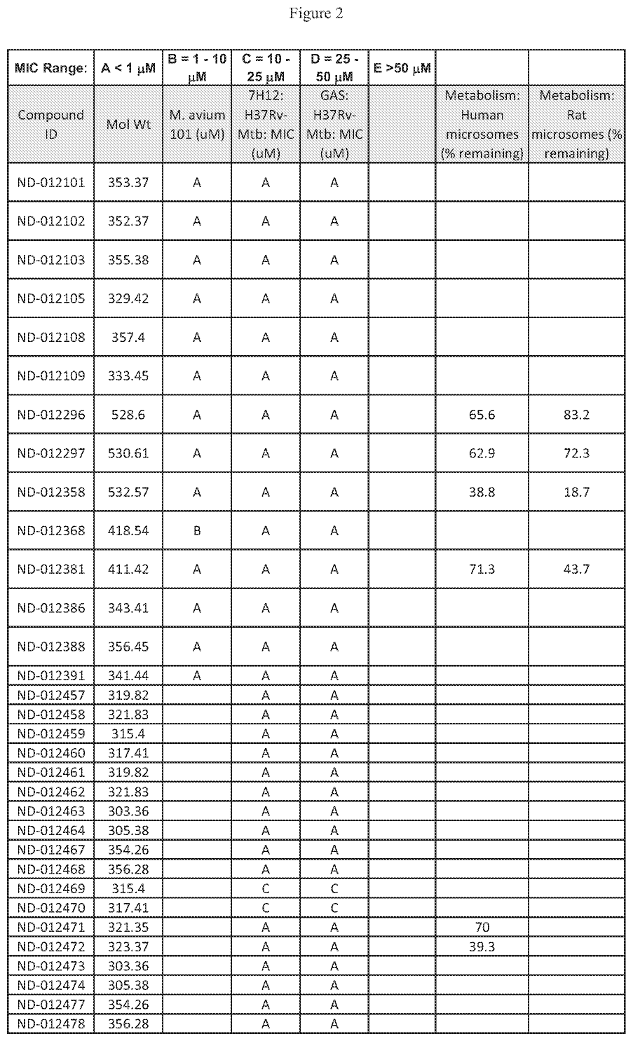

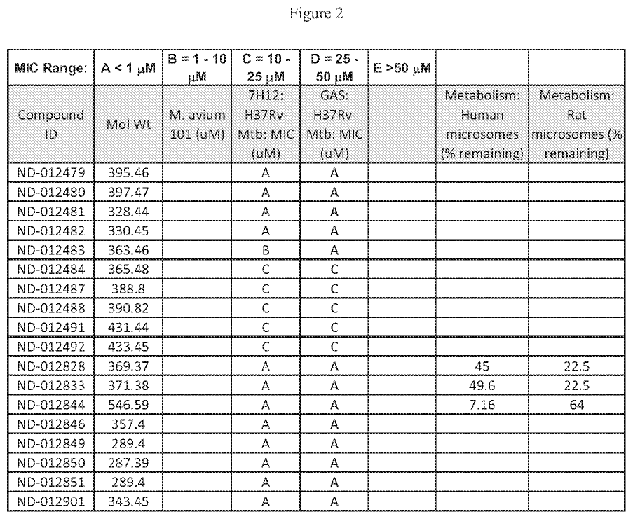

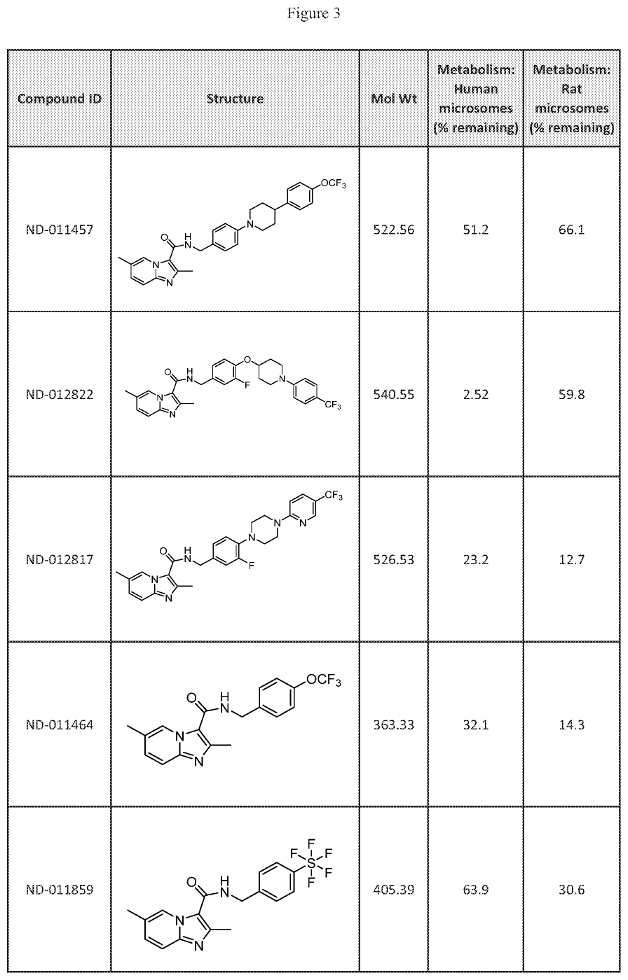

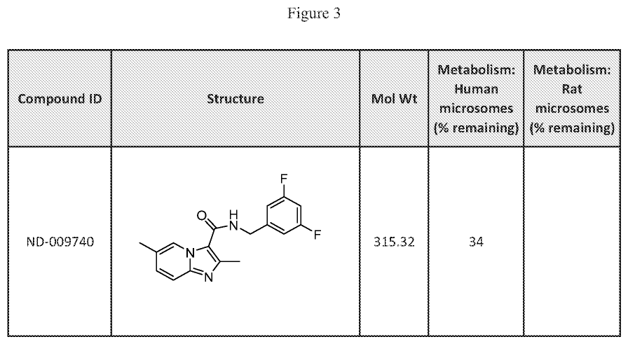

SAR (FIG. 2) demonstrates potency against mycobacteria and improved metabolic stability of the deuterated analogs. A comparison of the non-deuterated analogs is shown in FIG. 3 (stability comparison). The observed activity and biological properties demonstrate potent activity against mycobacteria and improved metabolic stability for the deuterated analogs compared to the non-deuterated ones.

FIG. 2 contains the Minimum Inhibitory Concentrations (MIC) of compounds screened against Mycobacterium tuberculosis strain H37Rv (H37Rv-Mtb) in two different media (7H12 and GAS) by the MABA assay and against Mycobacterium avium 101 (serotype 1). The MABA Mtb assay is described in Cho S, Lee H S, Franzblau S. Microplate alamar blue assay (MABA) and low oxygen recovery assay (LORA) for Mycobacterium tuberculosis. In Mycobacteria Protocols 2015 (pp. 281-292). Humana Press, New York, N.Y. The M. avium 101 screening assay is described Moraski G C, Cheng Y, Cho S, Cramer J W, Godfrey A, Masquelin T, Franzblau S G, Miller M J, Schorey J. Imidazo [1,2-a] pyridine-3-carboxamides are active antimicrobial agents against Mycobacterium avium infection in vivo. Antimicrobial agents and chemotherapy. 2016 Aug. 1; 60(8):5018-22. Additionally, the metabolic stability of these compounds was determined in human and rat microsomes by standard protocols and expressed as percent (%) of compound remaining after incubation (30 min).

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

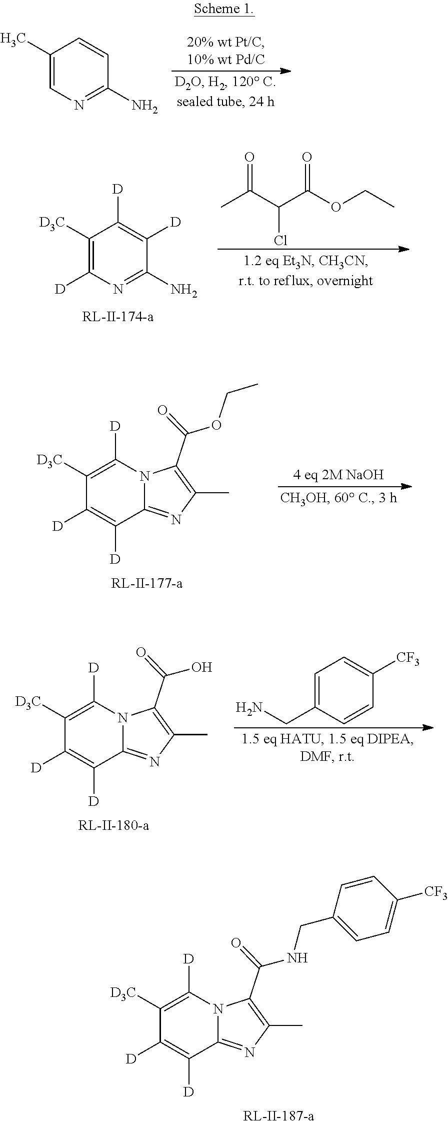

5-(Methyl-d₃)pyridin-3,4,6-d₃-2-amine (RL-II-174-a). (Bijani, S.; Jain, V.; Padmanbhan, D.; Pandey, B.; Shah, A. Mixed Pd/C and Pt/C as efficient catalysts for deuteration of mesalamine. Tetrahedron Lett. 2015, 56, 1211-1214). To a solution of 5-methylpyridin-2-amine (6 mmol, 648 mg) in 10 mL of D₂O (D, 99.9%) under an Argon atmosphere in a sealed tube was added 10% Pd/C (10% wt of 5-methylpyridin-2-amine, 65 mg) and 5% Pt/C (20% wt of 5-methylpyridin-2-amine, 130 mg). The tube was sealed with a rubber septum and filled with H₂ using three vacuum/H₂ cycles, then changed the rubber stopper to a polytetrafluoroethylene front seal plug. The reaction mixture was heated at 120° C. for 24 h. After cooling to room temperature, the mixture was diluted with EtOAc, and then filtered to remove the catalyst. The filtrate was partitioned between EtOAc and aqueous layers. The aqueous layer was extracted with EtOAc (2×15 mL). The combined organic layers were dried over Na₂SO₄, filtered, and concentrated in vacuo to give RL-II-174-a as a colorless solid in 85% yield (5.1 mmol, 581.4 mg). RL-II-174-a was used directly for the next step without any further purification.
Ethyl 2-methyl-6-(methyl-d₃)imidazo[1,2-a]pyridine-3-carboxylate-5,7,8-d₃ (RL-II-177-a). To a stirred mixture of RL-II-174-a (2 mmol, 228 mg), Et₃N (2.4 mmol, 326 μL) in 20 mL of CH₃CN was added dropwise a solution of ethyl 2-chloroacetoacetate (2.4 mmol, 333 μL) in 5 mL of CH₃CN over a period of 10 min. The mixture was stirred overnight at reflux, cooled to room temperature and filtered. The filtrate was concentrated under reduced pressure. The residue was purified by silica gel column chromatography, eluting with hexanes and EtOAc (3/1, v/v) to give RL-II-177-a as a white solid in 73% yield (1.46 mmol, 327 mg). ¹H NMR (400 MHz, MeOD): δ 1.42 (t, 3H, J=8.0 Hz), 2.34-2.37 (m, 0.16H), 2.63 (s, 3H), 4.40 (q, 2H, J=8.0 Hz), 7.37-7.39 (m, 0.07H), 7.45-7.47 (m, 0.05H), 9.09 (s, 0.05H). LC-MS: [M+H]⁺=225.20.
Note: Using DME as a solvent was not appropriate since DME can react with RL-II-174-a to give a byproduct as a major product. The desired compound RL-II-177-a was then obtained in very low yield.
2-Methyl-6-(methyl-d₃)imidazo[1,2-a]pyridine-3-carboxylic-5,7,8-d₃ acid (RL-II-180-a). To a solution of RL-II-177-a (1 mmol, 224 mg) in MeOH was added 2M NaOH (4eq, 2 mL). The mixture was stirred at 60° C. for 2 h, and then concentrated under reduced pressure. The residue was dissolved in water and washed with EtOAc. The aqueous layer was adjusted to pH 5.5-6.0 with 2N HCl at room temperature, and then concentrated under reduced pressure to get RL-II-180-a as a white solid used for the next step without further purification. LC-MS: [M+H]⁺=197.14.
2-Methyl-6-(methyl-d₃)—N-(4-(trifluoromethyl)benzyl)imidazo[1,2-a]pyridine-5,7,8-d₃-3-carboxamide (RL-II-187-a). To a mixture of RL-II-180-a (1 mmol, 196 mg), (4-(trifluoromethyl)phenyl)methanamine (1.2 mmol, 210 mg) and HATU (1.5 mmol, 570 mg) in 8 mL of anhydrous DMF was added DIPEA (1.5 mmol, 261 μL). The solution was stirred for 2 h at room temperature, and then concentrated under reduced pressure. The residue was purified by silica gel column chromatography, eluting with DCM and MeOH (50/1, v/v) to give RL-II-187-a (264 mg, 0.75 mmol) as a white solid in 75% yield. ¹H NMR (400 MHz, MeOD): δ 2.33 (s, 0.22H), 2.63 (s, 3H), 4.71 (s, 2H), 7.33 (s, 0.08H), 7.45 (s, 0.07H), 7.59 (d, 2H, J=8.0 Hz), 7.65 (d, 2H, J=8.0 Hz), 8.86 (s, 0.06H). LC-MS: [M+H]⁺=354.21.

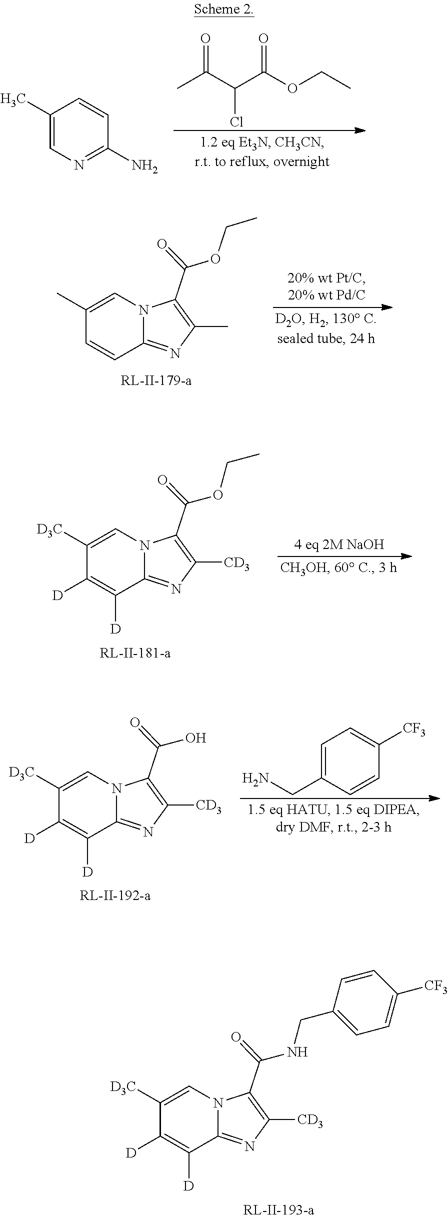

Ethyl 2,6-dimethylimidazo[1,2-a]pyridine-3-carboxylate(RL-II-179-a). To a stirred mixture of 5-methylpyridin-2-amine (1 mmol, 108 mg), Et₃N (1.2 mmol, 167 μL) in 10 mL of CH₃CN was added dropwise a solution of ethyl 2-chloroacetoacetate (1.2 mmol, 166 μL) in 5 mL of CH₃CN over a period of 10 min. The mixture was stirred overnight at reflux, cooled to room temperature and filtered. The filtrate was concentrated under reduced pressure. The residue was purified by silica gel column chromatography, eluting with hexanes and EtOAc (3/1, v/v) to give RL-II-179-a as a white solid in 70% yield (0.7 mmol, 153 mg). ¹H NMR (400 MHz, MeOD): δ 1.43 (t, 3H, J=8.0 Hz), 2.40 (s, 3H), 2.64 (s, 3H), 4.42 (q, 2H, J=8.0 Hz), 7.40 (s, 1H), 7.48 (m, 1H), 9.10 (s, 1H).
Ethyl 2,6-bis(methyl-d₃)imidazo[1,2-a]pyridine-3-carboxylate-7,8-d₂ (RL-II-181-a). To a solution of RL-II-179-a (1 mmol, 218 mg) in 5 mL of D₂O (D, 99.9%) under an Argon atmosphere in a sealed tube was added 10% Pd/C (20% wt of RL-II-179-a, 42 mg) and 20% Pt/C (20% wt of RL-II-179-a, 42 mg). The tube was sealed with a rubber septum and filled with H₂ using three vacuum/H₂ cycles, then changed the rubber septum to a polytetrafluoroethylene front seal plug. The reaction mixture was heated at 130° C. for 24 h. After cooling to room temperature, the mixture was diluted with EtOAc, and then filtered to remove the catalyst. The filtrate was partitioned between EtOAc and aqueous layers. The aqueous layer was extracted with EtOAc (2×15 mL). The combined organic layers were dried over Na₂SO₄, filtered, and concentrated in vacuo. The residue was purified by silica gel column chromatography, eluting with hexanes and EtOAc (4/1, v/v) to give RL-II-181-a as a white solid in 60% yield (0.6 mmol, 136 mg). ¹H NMR (400 MHz, MeOD): δ 1.43 (t, 3H, J=8.0 Hz), 2.36 (s, 0.06H), 2.59-2.60 (s, 0.06H), 4.41 (q, 2H, J=8.0 Hz), 7.39 (s, 0.02H), 7.47 (s, 0.02H), 9.11 (s, 0.57H). LC-MS: [M+H]⁺=227.16.
2,6-Bis(methyl-d₃)imidazo[1,2-a]pyridine-3-carboxylic-7,8-d₂ acid (RL-II-192-a). RL-II-192-a was synthesized according to the method for RL-II-180-a. Starting with RL-II-181-a (1 mmol, 226 mg), the compound RL-II-192-a was obtained as a white solid and used for the next step without further purification.
2,6-Bis(methyl-d₃)—N-(4-(trifluoromethyl)benzyl)imidazo[1,2-a]pyridine-7,8-d₂-3-carboxamide (RL-II-193-a). To a mixture of RL-II-192-a (1 mmol, 198 mg), (4-(trifluoromethyl)phenyl)methanamine (1.2 mmol, 210 mg) and HATU (1.5 mmol, 570 mg) in 8 mL of anhydrous DMF was added DIPEA (1.5 mmol, 261 μL). The solution was stirred for 2 h at room temperature, and then concentrated under reduced pressure. The residue was purified by silica gel column chromatography, eluting with DCM and MeOH (50/1, v/v) to give RL-II-193-a (195 mg, 0.55 mmol) as a white solid in 55% yield. ¹H NMR (400 MHz, MeOD): δ 2.37 (s, 0.07H), 2.63 (s, 0.06H), 4.71 (s, 2H), 7.62 (d, 2H, J=8.0 Hz), 7.66 (d, 2H, J=8.0 Hz), 8.92 (s, 0.47H). LC-MS: [M+H]⁺=356.24

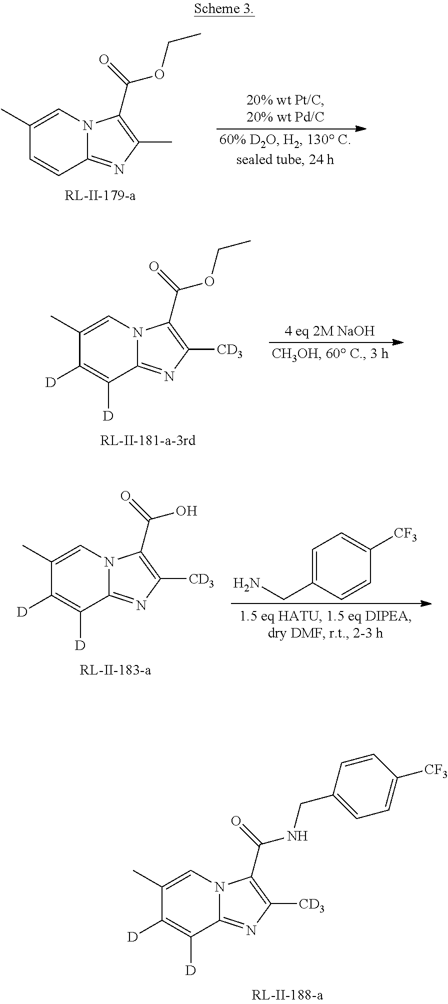 [SMILES: CCOC(=O)c1c(C)nc2ccc(C)cn12 ; [2H]c1c(C)cn2c(C(=O)OCC)c(C([2H])([2H])[2H])nc2c1[2H] ; [2H]c1c(C)cn2c(C(=O)O)c(C([2H])([2H])[2H])nc2c1[2H] ; NCc1ccc(C(F)(F)F)cc1 ; [2H]c1c(C)cn2c(C(=O)NCc3ccc(C(F)(F)F)cc3)c(C([2H])([2H])[2H])nc2c1[2H]]

Ethyl 6-methyl-2-(methyl-d)imidazo[1,2-a]pyridine-3-carboxylate-7,8-d₂ (RL-II-181-α-3rd). RL-II-181-a-3rd was synthesized according to the method for RL-II-181-a by using D₂O (D, 70%) as the solvent. RL-II-181-α-3rd was obtained as a white solid in 53% yield. ¹H NMR (400 MHz, MeOD): δ 1.43 (t, 3H, J=8.0 Hz), 2.38-2.40 (m, 2H), 2.65-2.66 (m, 0.56H), 4.42 (q, 2H, J=8.0 Hz), 7.40-7.42 (m, 0.19H), 7.48-7.50 (m, 0.18H), 9.15 (s, 0.68H). LC-MS: [M+H]+=224.22.
6-Methyl-2-(methyl-d₃)imidazo[1,2-a]pyridine-3-carboxylic-7,8-d₂ acid (RL-II-183-a). RL-II-183-a was prepared according to the method for RL-II-192-a. Starting from RL-II-181-α-3rd, RL-II-183-a was obtained as a white solid used for the next step without further purification. LC-MS: [M+H]⁺=196.17.
6-Methyl-2-(methyl-d₃)—N-(4-(trifluoromethyl)benzyl)imidazo[1,2-a]pyridine-7,8-d₂-3-carboxamide (RL-II-188-a). RL-II-188-a was prepared according to the method for RL-II-187-a. Starting from RL-II-183-a (1 mmol, 195 mg), RL-II-188-a was obtained as a white solid in 61% yield (0.61 mmol, 214 mg). ¹H NMR (400 MHz, MeOD): δ 2.34-2.36 (m, 2.19H), 2.59 (s, 0.15H), 4.70 (s, 2H), 7.33 (s, 0.06H), 7.45 (s, 0.08H), 7.58 (d, 2H, J=8.0 Hz), 7.65 (d, 2H, J=8.0 Hz), 8.86 (s, 0.66H). LC-MS: [M+H]⁺=353.30.

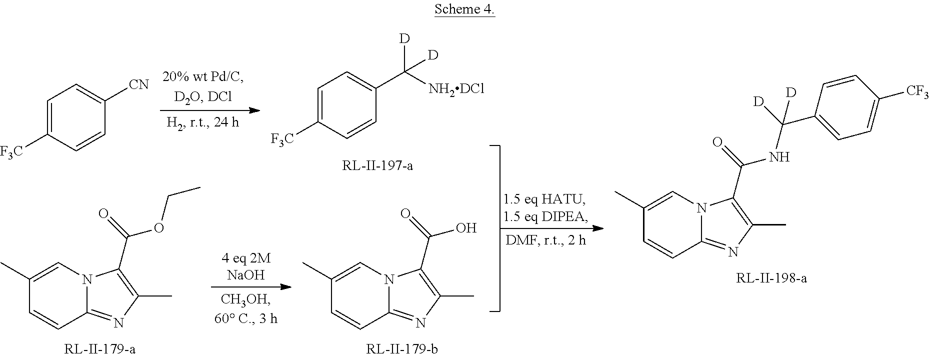

(4-(Trifluoromethyl)phenyl)methan-d₂-amine DCl salt (RL-II-197-a). (Kurita, T.; Aoki, F.; Mizumoto, T.; Maejima, T.; Esaki, H.; Maegawa, T.; Monguchi, Y.; Sajiki, H. Facile and convenient method of deuterium gas generation using a Pd/C-catalyzed H2-D2 exchange reaction and its application to synthesis of deuterium-labeled compounds. Chem. Eur. J. 2008, 14, 3371-3397). 10% Pd/C (34 mg, 20% wt of 4-(trifluoromethyl)benzonitrile) and 5 mL of D₂O were placed in a 100 mL round-bottom flask. The system was sealed with a rubber septum and filled with H₂ using five vacuum/H₂ cycles. The mixture was stirred at room temperature for 24 h, then 4-(trifluoromethyl)benzonitrile (1 mmol, 171 mg) was added followed by 1.5 mmol (160 μL) of deuterium chloride (D, 99.5%, DCl 35% in D₂O). The mixture was stirred at room temperature overnight and then filtered to remove the catalyst. The residue was concentrated under reduced pressure to give RL-II-197-a as a white solid in 80% yield (171 mg, 0.8 mmol) used for the next step without further purification. ¹H NMR (400 MHz, MeOD): δ 4.22 (s, 0.2H), 7.68 (brs, 2H), 7.77 (brs, 2H).
2,6-Dimethylimidazo[1,2-a]pyridine-3-carboxylic acid (RL-II-179-b). RL-II-179-b was synthesized according to the method for RL-II-180-a. Starting with RL-II-179-a, RL-II-179-b was obtained as a white solid and used directly for the next step.
2,6-Dimethyl-N-((4-(trifluoromethyl)phenyl)methyl-d₂)imidazo[1,2-a]pyridine-3-carboxamide (RL-II-198-a). To a mixture of RL-II-179-b (0.63 mmol, 120 mg), RL-II-197-a (0.63 mmol, 150 mg) and HATU (0.95 mmol, 359 mg) in 5 mL of anhydrous DMF was added DIPEA (1.58 mmol, 275 μL). The solution was stirred for 1 h at room temperature, and then concentrated under reduced pressure. The residue was purified by silica gel column chromatography, eluting with DCM and MeOH (50/1, v/v) to give RL-II-198-a (65 mg, 0.19 mmol) as a white solid in 30% yield.
¹H NMR (400 MHz, DMSO-d₆): δ 2.31 (s, 3H), 2.60 (s, 3H), 4.59-4.60 (m, 0.2H), 7.26 (d, 1H, J=8.0 Hz), 7.49 (d, 1H, J=8.0 Hz), 7.59 (d, 2H, J=8.0 Hz), 7.71 (d, 2H, J=8.0 Hz), 8.37 (s, 1H), 8.87 (s, 1H). LC-MS: [M+H]⁺=350.29.

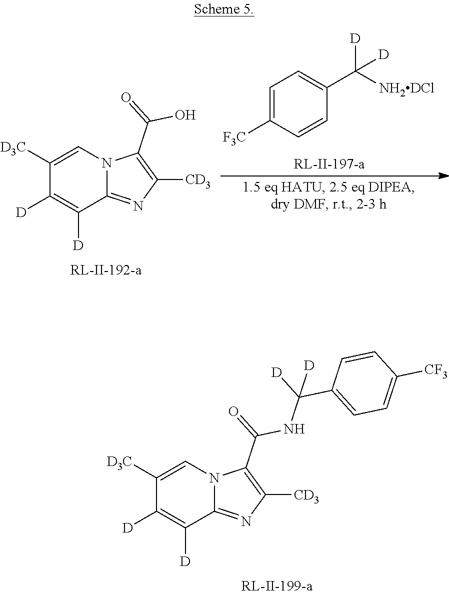

2,6-Bis(methyl-d₃)-N-((4-(trifluoromethyl)phenyl)methyl-d₂)imidazo[1,2-a]pyridine-7,8-d₂-3-carboxamide (RL-II-199-a). To a mixture of RL-II-192-a (1 mmol, 198 mg), RL-II-197-a (1 mmol, 214 mg) and HATU (1.5 mmol, 570 mg) in 10 mL of anhydrous DMF was added DIPEA (2.5 mmol, 276 μL). The solution was stirred for 2 h at room temperature, and then concentrated under reduced pressure. The residue was purified by silica gel column chromatography, eluting with DCM and MeOH (50/1, v/v) to give RL-II-199-a (200 mg, 0.56 mmol) as a white solid in 56% yield. ¹H NMR (400 MHz, DMSO-d₆): δ 2.31 (s, 0.11H), 2.59 (s, 0.11H), 4.60 (d, 0.18H, J=4.0 Hz), 7.60 (d, 2H, J=8.0 Hz), 7.71 (d, 2H, J=8.0 Hz), 8.54 (s, 1H), 8.91 (s, 0.62H). LC-MS: [M+H]⁺=358.30.

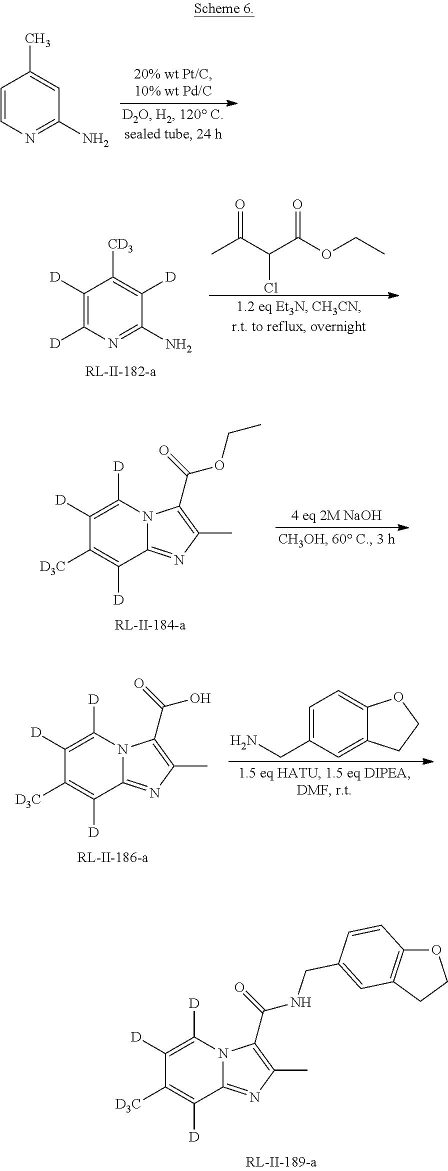

4-(Methyl-d3)pyridin-3,5,6-d₃-2-amine (RL-II-182-a). To a solution of 4-methylpyridin-2-amine (4 mmol, 432 mg) in 6 mL of D₂O (D, 99.9%) under an Argon atmosphere in a sealed tube was added 10% Pd/C (10% wt of 4-methylpyridin-2-amine, 43 mg) and 5% Pt/C (20% wt of 4-methylpyridin-2-amine, 86 mg). The tube was sealed with a rubber septum and filled with H₂ using three vacuum/H₂ cycles, then changed the rubber septum to a polytetrafluoroethylene front seal plug. The reaction mixture was heated at 120° C. for 24 h. After cooling to room temperature, the mixture was diluted with EtOAc, and then filtered to remove the catalyst. The filtrate was partitioned between EtOAc and aqueous layers. The aqueous layer was extracted with EtOAc (2×15 mL). The combined organic layers were dried over Na₂SO₄, filtered, and concentrated in vacuo to give RL-II-182-a as a colorless solid in 88% yield (3.5 mmol, 400 mg). RL-II-182-a was used directly for the next step without any further purification.
Ethyl 2-methyl-7-(methyl-d₃)imidazo[1,2-a]pyridine-3-carboxylate-5,6,8-d₃ (RL-II-184-a). To a stirred mixture of RL-II-182-a (3.5 mmol, 399 mg), Et₃N (4.2 mmol, 570) in 30 mL of CH₃CN was added dropwise a solution of ethyl 2-chloroacetoacetate (4.2 mmol, 583 μL) in 5 mL of CH₃CN over a period of 10 min. The mixture was stirred overnight at reflux, cooled to room temperature and filtered. The filtrate was concentrated under reduced pressure. The residue was purified by silica gel column chromatography, eluting with hexanes and EtOAc (3/1, v/v) to give RL-II-184-a as a white solid in 70% yield (2.45 mmol, 549 mg). ¹H NMR (400 MHz, MeOD): δ 1.42 (t, 3H, J=8.0 Hz), 2.41 (s, 0.19H), 2.60-2.62 (m, 3H), 4.39 (q, 2H, J=8.0 Hz), 6.96 (s, 0.06H), 7.34 (m, 0.1H), 9.14 (s, 0.04H).
2-Methyl-7-(methyl-d)imidazo[1,2-a]pyridine-3-carboxylic-5,6,8-d₃ acid (RL-II-186-a). To a solution of RL-II-184-a (1.8 mmol, 410 mg) in MeOH was added 2M NaOH (4eq, 4 mL). The mixture was refluxed under 60° C. for 2 h, and then concentrated under reduced pressure. The residue was dissolved in water and washed with EtOAc. The aqueous layer was adjusted to pH 5.5-6.0 with 2N HCl at room temperature, and then concentrated under reduced pressure to give RL-II-186-a as a white solid that was used for the next step without further purification.
N-((2,3-dihydrobenzofuran-5-yl)methyl)-2-methyl-7-(methyl-d₃)imidazo[1,2-a]pyridine-5,6,8-d₃-3-carboxamide (RL-II-189-a). RL-II-186-a (1 mmol, 196 mg), (2,3-dihydrobenzofuran-5-yl)methanamine (1.2 mmol. 179 mg), and HATU (1.5 mmol, 570 mg) were dissolved in 8 mL of dry DMF at room temperature. To the mixture was added DIPEA (1.5 mmol, 261 μL), and then stirred for 1 h. The resulting solution was concentrated under reduced pressure and the residue was purified by silica gel column chromatography, eluting with DCM and MeOH (100/1, v/v) to give RL-II-189-a (199 mg, 0.61 mmol) as a white solid in 61% yield. ¹H NMR (400 MHz, MeOD): δ 2.40 (s, 0.3H), 2.57 (s, 3H), 3.19 (t, 2H, J=8.0 Hz), 4.50-4.52 (m, 4H), 6.68 (d, 1H, J=8.0 Hz), 6.90 (s, 0.07H), 7.12 (d, 1H, J=8.0 Hz), 7.26 (s, 1H), 7.31 (s, 0.15H), 8.90 (s, 0.04H). LC-MS: [M+H]⁺=328.38.

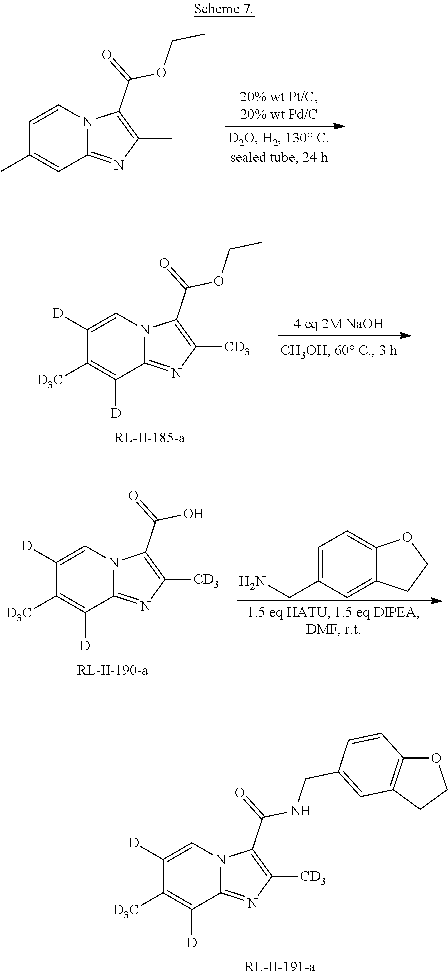 [SMILES: CCOC(=O)c1c(C)nc2cc(C)ccn12 ; [2H]c1cn2c(C(=O)OCC)c(C([2H])([2H])[2H])nc2c([2H])c1C([2H])([2H])[2H] ; [2H]c1cn2c(C(=O)O)c(C([2H])([2H])[2H])nc2c([2H])c1C([2H])([2H])[2H] ; NCc1ccc2c(c1)CCO2 ; [2H]c1cn2c(C(=O)NCc3ccc4c(c3)CCO4)c(C([2H])([2H])[2H])nc2c([2H])c1C([2H])([2H])[2H]]

Ethyl 2,7-bis(methyl-d3)imidazo[1,2-a]pyridine-3-carboxylate-6,8-d₂ (RL-II-185-a). To a solution of ethyl 2,7-dimethylimidazo[1,2-a]pyridine-3-carboxylate (2 mmol, 436 mg) in 10 mL of D₂O (D, 99.9%) under an Argon atmosphere in a sealed tube was added 10% Pd/C (20% wt of ethyl 2,7-dimethylimidazo[1,2-a]pyridine-3-carboxylate, 86 mg) and 20% Pt/C (20% wt of ethyl 2,7-dimethylimidazo[1,2-a]pyridine-3-carboxylate, 86 mg). The tube was sealed with a rubber septum and filled with H₂ using three vacuum/H₂ cycles, then changed the rubber septum to a polytetrafluoroethylene front seal plug. The reaction mixture was heated at 130° C. for 24 h. After cooling to room temperature, the mixture was diluted with EtOAc, and then filtered to remove the catalyst. The filtrate was partitioned between EtOAc and aqueous layers. The aqueous layer was extracted with EtOAc (2×15 mL). The combined organic layers were dried over Na₂SO₄, filtered, and concentrated in vacuo. The residue was purified by silica gel column chromatography, eluting with hexanes and EtOAc (4/1, v/v) to give RL-II-185-a as a white solid in 52% yield (1.04 mmol, 235 mg). LC-MS: [M+H]+=227.20.
2,7-Bis(methyl-d₃)imidazo[1,2-a]pyridine-3-carboxylic-6,8-d₂ acid (RL-II-190-a). RL-II-190-a was synthesized according to the method for RL-II-186-a. Starting with RL-II-185-a, RL-II-190-a was obtained as a white solid and used directly for the next step. LC-MS: [M+H]⁺=199.31.
N-((2,3-dihydrobenzofuran-5-yl)methyl)-2,7-bis(methyl-d)imidazo[1,2-a]pyridine-6,8-d₂-3-carboxamide (RL-II-191-a). RL-II-191-a was synthesized according to the method for RL-II-189-a. Starting with RL-II-190, RL-II-191-a was obtained as a white solid in 48% yield (0.48 mmol, 158 mg). ¹H NMR (400 MHz, MeOD): δ 2.40 (s, 0.3H), 2.57 (s, 3H), 3.19 (t, 2H, J=8.0 Hz), 4.50-4.52 (m, 4H), 6.67 (d, 1H, J=12.0 Hz), 6.89 (d, 0.1H, J=8.0 Hz), 7.11 (d, 1H, J=12.0 Hz), 7.26 (s, 1H), 7.31 (s, 0.08H), 8.90 (s, 0.78H). LC-MS: [M+H]⁺=330.27.

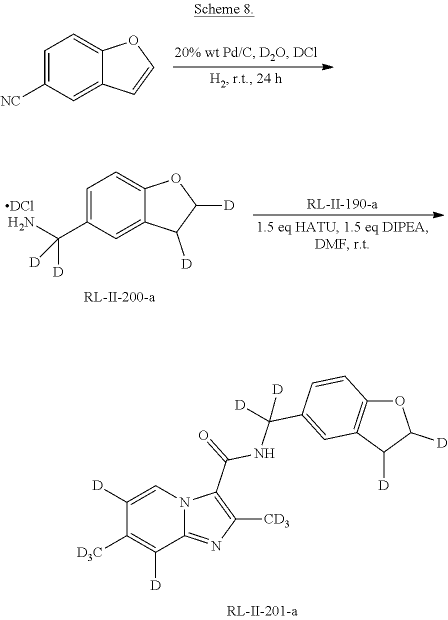

(2,3-Dihydrobenzofuran-5-yl-2,3-d₂)methan-d₂-amine DCl salt (RL-II-200-a). 10% Pd/C (29 mg, 20% wt of benzofuran-5-carbonitrile) and 5 mL of D₂O were placed in a 100 mL round-bottom flask. The system was sealed with a rubber septum and filled with H₂ using five vacuum/H₂ cycles. The mixture was stirred at room temperature for 24 h, then the solution of benzofuran-5-carbonitrile (1 mmol, 143 mg) in 3 mL of CD₃OD was added followed by 1.5 mmol (160 μL) deuterium chloride (D, 99.5%, DCl 35% in D₂O). The mixture was stirred at room temperature overnight and then filtered to remove the catalyst. The residue was concentrated under reduced pressure to give RL-II-200-a as a white solid in 78% yield (148 mg, 0.78 mmol) used for the next step without further purification. ¹H NMR (400 MHz, DMSO-d₆): δ 3.13-3.19 (m, 1.32H), 3.86-3.90 (m, 0.4H), 4.50-4.56 (m, 1.16H), 6.77 (d, 1H, J=8.0 Hz), 7.19 (d, 1H, J=8.0 Hz), 7.35 (s, 1H), 8.28 (s, 2H).
N-((2,3-Dihydrobenzofuran-5-yl-2,3-d₂)methyl-d₂)-2,7-bis(methyl-d₃)imidazo[1,2-a]pyridine-6,8-d₂-3-carboxamide (RL-II-201-a). RL-II-190-a (0.5 mmol, 99 mg), RL-II-200-a (0.6 mmol. 114 mg), and HATU (1 mmol, 380 mg) were dissolved in 8 mL of dry DMF at room temperature. To the mixture was added DIPEA (1 mmol, 174 μL), and then stirred for 1 h. The resulting solution was concentrated under reduced pressure and the residue was purified by silica gel column chromatography, eluting with DCM and MeOH (100/1, v/v) to give RL-II-201-a (100 mg, 0.6 mmol) as a white solid in 60% yield. ¹H NMR (400 MHz, DMSO-d₆): δ 2.37 (s, 0.10H), 3.13 (t, 1.2H, J=8.0 Hz), 4.40 (d, 0.23H, J=4.0 Hz), 4.46-4.52 (m, 1.05H), 6.70 (d, 1H, J=8.0 Hz), 6.85 (d, 0.09H, J=4.0 Hz), 7.08 (d, 1H, J=8.0 Hz), 7.24 (s, 1H), 7.34 (s, 1H), 8.15 (s, 1H), 8.93 (s, 0.53H). LC-MS: [M+H]⁺=334.33.

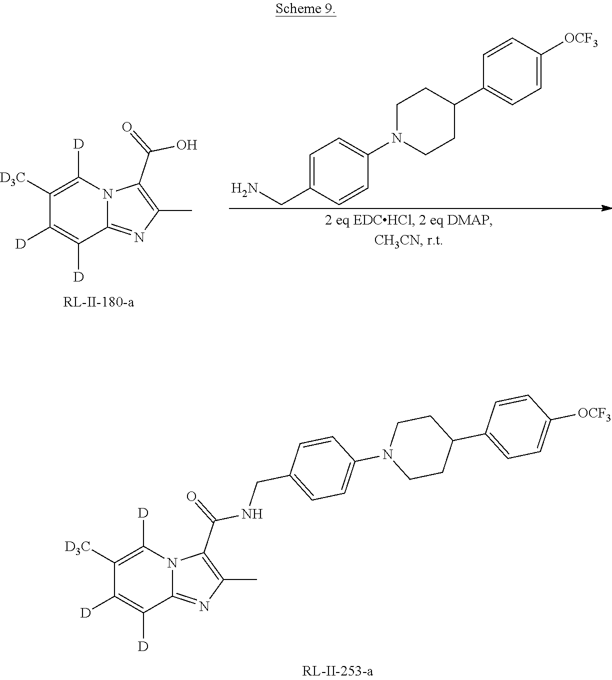 [SMILES: [2H]c1c(C([2H])([2H])[2H])c([2H])n2c(C(=O)O)c(C)nc2c1[2H] ; NCc1ccc(N2CCC(c3ccc(OC(F)(F)F)cc3)CC2)cc1 ; [2H]c1c(C([2H])([2H])[2H])c([2H])n2c(C(=O)NCc3ccc(N4CCC(c5ccc(OC(F)(F)F)cc5)CC4)cc3)c(C)nc2c1[2H]]

2-Methyl-6-(methyl-d3)-N-(4-(4-(4-(trifluoromethoxy)phenyl)piperidin-1-yl)benzyl)imidazo[1,2-a]pyridine-5,7,8-d3-3-carboxamide (RL-II-253-a). To a mixture of RL-II-180-a (0.157 mmol, 31 mg) and (4-(4-(4-(trifluoromethoxy)phenyl)piperidin-1-yl)phenyl)methanamine (0.14 mmol, 50 mg), in 5 mL of anhydrous CH₃CN was added EDC.HCl (0.28 mmol, 54 mg) and DMAP (0.28 mmol, 34 mg). The solution was stirred overnight at room temperature, and then concentrated under reduced pressure. The residue was purified by silica gel column chromatography, eluting with DCM and MeOH (50/1, v/v) to give RL-II-253-a (0.066 mmol, 35 mg) as a white solid. ¹H-NMR (CDCl₃, 400 MHz): δ 1.83-1.97 (m, 4H), 2.33 (s, 0.17H), 2.65-2.70 (m, 4H), 2.83 (t, 2H, J=12.0 Hz), 3.80 (d, 2H, J=12.0 Hz), 4.61 (d, 2H, J=8.0 Hz), 5.98 (s, 1H), 6.97 (d, 2H, J=8.0 Hz), 7.15 (d, 2H, J=8.0 Hz), 7.25-7.31 (m, 4H), 7.47 (s, 0.05 Hz), 7.52 (s, 0.02 Hz), 9.25 (s, 0.03H).

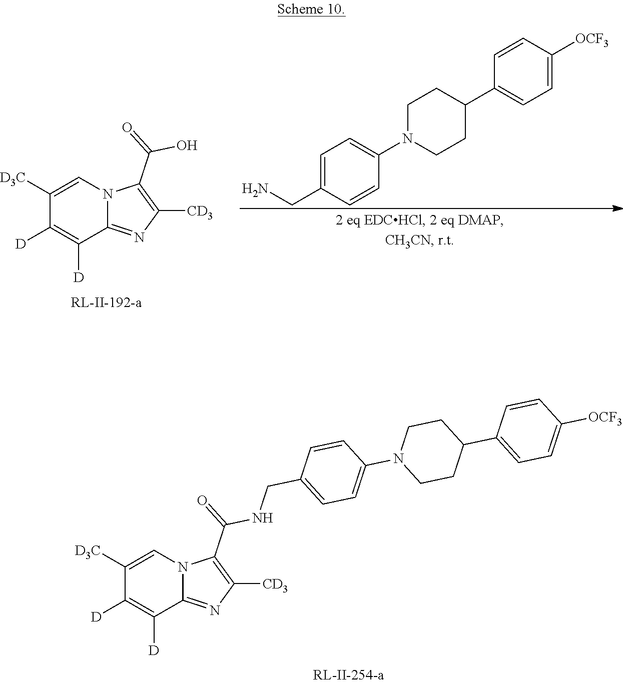 [SMILES: [2H]c1c(C([2H])([2H])[2H])cn2c(C(=O)O)c(C([2H])([2H])[2H])nc2c1[2H] ; NCc1ccc(N2CCC(c3ccc(OC(F)(F)F)cc3)CC2)cc1 ; [2H]c1c(C([2H])([2H])[2H])cn2c(C(=O)NCc3ccc(N4CCC(c5ccc(OC(F)(F)F)cc5)CC4)cc3)c(C([2H])([2H])[2H])nc2c1[2H]]

2,6-Bis(methyl-d₃)—N-(4-(4-(4-(trifluoromethoxy)phenyl)piperidin-1-yl)benzyl)imidazo[1,2-a]pyridine-7,8-d₂-3-carboxamide (RL-II-254-a). To a mixture of RL-II-192-a (0.157 mmol, 31 mg) and (4-(4-(4-(trifluoromethoxy)phenyl)piperidin-1-yl)phenyl)methanamine (0.14 mmol, 50 mg), in 5 mL of anhydrous CH₃CN was added EDC.HCl (0.28 mmol, 54 mg) and DMAP (0.28 mmol, 34 mg). The solution was stirred overnight at room temperature, and then concentrated under reduced pressure. The residue was purified by silica gel column chromatography, eluting with DCM and MeOH (50/1, v/v) to give RL-II-254-a (0.057 mmol, 30 mg) as a white solid. ¹H-NMR (CDCl₃, 400 MHz): δ 1.82-1.97 (m, 4H), 2.33 (s, 0.15H), 2.63-2.70 (m, 1H), 2.83 (td, 2H, J₁=12.0 Hz, J₂=4.0 Hz), 3.80 (d, 2H, J=12.0 Hz), 4.61 (d, 2H, J=8.0 Hz), 5.97 (s, 1H), 6.97 (d, 2H, J=8.0 Hz), 7.15 (d, 2H, J=8.0 Hz), 7.25-7.31 (m, 4H), 7.47 (s, 0.04H), 7.52 (s, 0.03H), 9.25 (s, 0.47H).

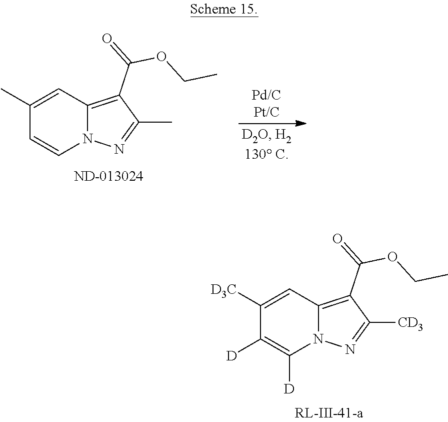 [SMILES: CCOC(=O)c1c(C)nn2ccc(C)cc12 ; [2H]c1c(C([2H])([2H])[2H])cc2c(C(=O)OCC)c(C([2H])([2H])[2H])nn2c1[2H]]

Ethyl 2,5-bis(methyl-d₃)pyrazolo[1,5-a]pyridine-3-carboxylate-6,7-d₂ (RL-III-41-a). To a solution of ND-013024 (0.92 mmol, 200 mg) in 10 mL of D₂O (D, 99.9%) under an Argon atmosphere in a sealed tube was added 10% Pd/C (20% wt of ND-013024, 40 mg) and 20% Pt/C (20% wt of ND-013024, 40 mg). The tube was sealed with a rubber septum and filled with H₂ using three vacuum/H₂ cycles, then the rubber septum was exchanged with a polytetrafluoroethylene front seal plug. The reaction mixture was heated at 130° C. for 14 h. After cooling to room temperature, the mixture was diluted with EtOAc, and then filtered to remove the catalyst. The filtrate was partitioned between EtOAc and aqueous layers. The aqueous layer was extracted with EtOAc (2×15 mL). The combined organic layers were dried over Na₂SO₄, filtered, and concentrated in vacuo. The residue was purified by silica gel column chromatography, eluting with hexanes and EtOAc (4/1, v/v) to give RL-III-41-a as a white solid in 50% yield. ¹H NMR (400 MHz, CDCl₃): δ 1.42 (t, 3H, J=8.0 Hz), 2.41 (s, 0.07H), 2.61 (s, 0.04H), 4.37 (q, 2H, J=8.0 Hz), 6.69 (s, 0.01H), 7.85 (s, 1H), 8.28 (s, 0.01H). LC-MS: [M+H]⁺=227.16.

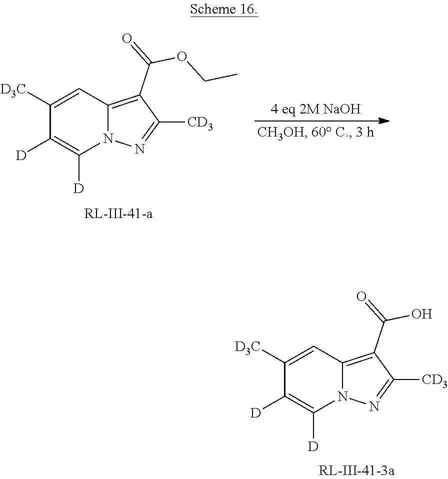 [SMILES: [2H]c1c(C([2H])([2H])[2H])cc2c(C(=O)OCC)c(C([2H])([2H])[2H])nn2c1[2H] ; [2H]c1c(C([2H])([2H])[2H])cc2c(C(=O)O)c(C([2H])([2H])[2H])nn2c1[2H]]

2,5-bis(methyl-d3)pyrazolo[1,5-a]pyridine-3-carboxylic-6,7-d2 acid (RL-III-41-3a). To a solution of RL-III-41-a (1.8 mmol, 410 mg) in MeOH was added 2M NaOH (4eq, 4 mL). The mixture was refluxed for 2 h, and then concentrated under reduced pressure. The residue was dissolved in water and washed with EtOAc. The aqueous layer was adjusted to pH 5.5-6.0 with 2N HCl at room temperature, and then concentrated under reduced pressure to get RL-III-41-3a as a white solid used for the next step without further purification. ¹H NMR (400 MHz, CD₃OD): δ 6.67 (s, 0.01H), 7.83 (s, 1H), 8.21 (s, 0.01H); MS: [M+H]⁺=199.00.

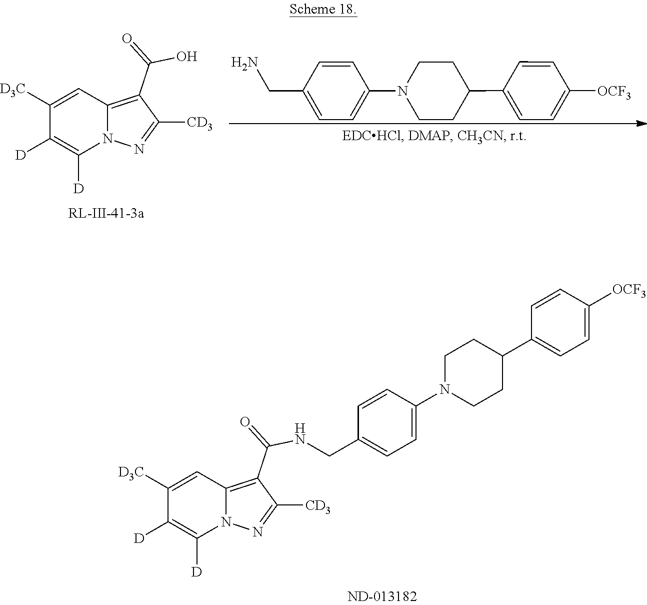 [SMILES: [2H]c1c(C([2H])([2H])[2H])cc2c(C(=O)O)c(C([2H])([2H])[2H])nn2c1[2H] ; NCc1ccc(N2CCC(c3ccc(OC(F)(F)F)cc3)CC2)cc1 ; [2H]c1c(C([2H])([2H])[2H])cc2c(C(=O)NCc3ccc(N4CCC(c5ccc(OC(F)(F)F)cc5)CC4)cc3)c(C([2H])([2H])[2H])nn2c1[2H]]

To a mixture of RL-III-41-3a (0.25 mmol, 50 mg) and (4-(4-(4-(trifluoromethoxy)phenyl)piperidin-1-yl)phenyl)methanamine (0.25 mmol, 93 mg), in 5 mL of anhydrous CH₃CN was added EDC•HCl (0.30 mmol, 58 mg) and DMAP (0.30 mmol, 37 mg). The solution was stirred overnight at room temperature, and then concentrated under reduced pressure. The residue was dissolved in CH₂Cl₂ and washed with 5% HOAc aq. soln. (2×), NaHCO₃ sat. soln. (2×), and brine. The combined organic layers were dried over Na₂SO₄, filtered, and concentrated in vacuo. The residue was purified by recrystallization from hot CH₃CN to give product (ND-013182) as an off white solid. ¹H NMR (500 MHz, CDCl₃) 2.02-1.84 (m, 4H), 2.69 (tt, J=12.0, 3.8 Hz, 1H), 2.85 (td, J=12.2, 2.7 Hz, 2H), 3.87-3.79 (m, 2H), 4.63 (d, J=5.5 Hz, 2H), 5.90 (t, J=5.4 Hz, 1H, NH₂), 7.03-6.97 (m, 2H), 7.21-7.16 (m, 2H), 7.31-7.25 (m, 2H), 7.36-7.31 (m, 2H), 7.96 (s, 1H).
Various pyrazolo[1,5-a]pyridine-4,6,7-d3-3-carboxamides can be prepared by the procedure of Scheme 19, beginning with deuteration of the 4-methylpyridine (CAS: 108-89-4) by the methods described previously. Pyrazolo[1,2-a]pyridine carboxamides can then be formed by the methods described in “Design, synthesis, and biological evaluation of pyrazolo [1,5-a]pyridine-3-carboxamides as novel antitubercular agents.” ACS Medicinal Chemistry Letters, 2015, 6(7), 814-818. The synthesis of 2-methyl-5-(methyl-d3)-N-(4-(trifluoromethyl)benzyl)pyrazolo[1,5-a]pyridine-4,6,7-d3-3-carboxamide is shown here as an example.

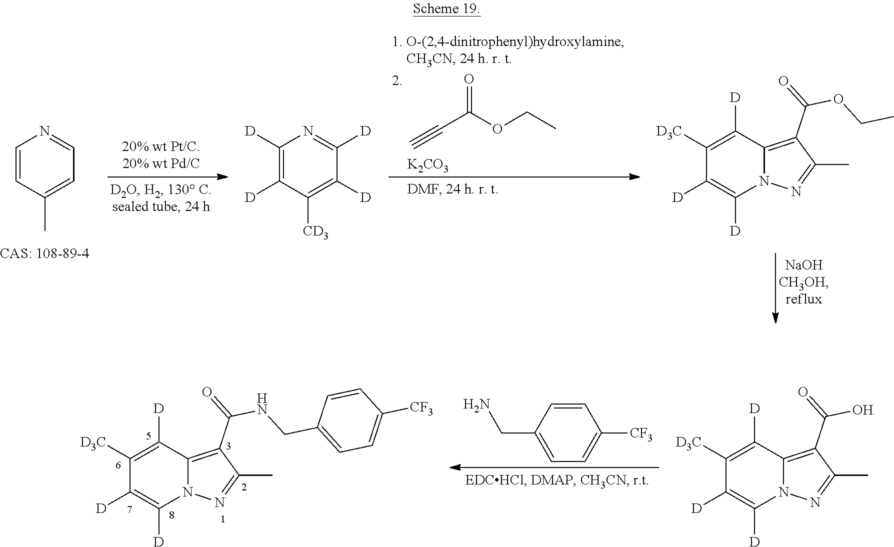

In vivo PK determination of ND-011176 (non-deuterated compound) and ND-012101 (deuterated compound) and in vivo efficacy determination of ND-012101 in a chronic murine infection model.

### Single Dose pK:

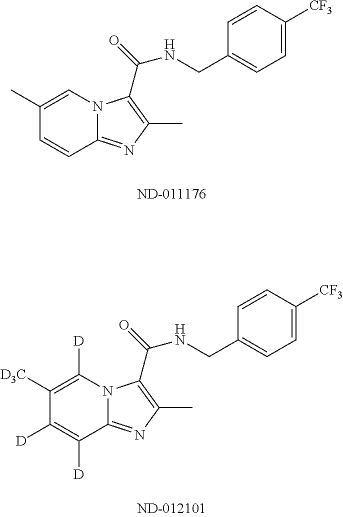 [SMILES: Cc1ccc2nc(C)c(C(=O)NCc3ccc(C(F)(F)F)cc3)n2c1 ; [2H]c1c(C([2H])([2H])[2H])c([2H])n2c(C(=O)NCc3ccc(C(F)(F)F)cc3)c(C)nc2c1[2H]]

Based on MIC and acute toxicity data, two compounds ND-011176 and a deuterated analog ND-012101 were selected for evaluation in a single dose pharmacokinetic study (with and without pre-dosing of 1-Aminobenzotriazole, ABT) after oral administration to female Balb/c mice.

All animals were administered with the prepared test article via oral gavage at a target dose level of 200 mg/kg and at the dose volume detailed in the table 1 below. Each animal in Groups 2, 4, 6, 8 and 10 received a dose of ABT (100 mg/kg) via oral gavage two hours prior to Test Article administration. Blood was collected at 0.5, 2, 4, 8, 12 and 24 hours except for groups 6 and 10 which showed adverse effects and were euthanized at approximately 5 hours after dosing.

TABLE 1
pK treatment administration

| | No. | Test | Dose | Dose | Dose | | |
|---|---|---|---|---|---|---|---|
| | of | Article | Level | Conc. | Volume | Dose | |
| Group | mice | (TA) | (mg/kg) | (mg/mL) | (mL/kg) | Vehicle | Route |
| 5 | 9 | TA 3 | 200 | 20 | 10 | TAs in 20% | PO (for |
| | | (ND- | | | | cyclodextrin | both TAs |
| | | 012101) | | | | ABT100 | and |
| 6 | 9 | TA 3 post | 200 | 20 | 10 | prepared in | ABT100) |
| | | ABT. ABT | (ABT100) | | | 0.5% (wt/vol) | |
| | | given 2 | | | | methylcellulose | |
| | | hours prior | | | | (400 cP) | |
| | | to TA | | | | | |
| 9 | 9 | TA 5 | 200 | 20 | 10 | TAs in 20% | |
| | | (ND- | | | | TPGS | |
| | | 011176) | | | | ABT100 | |
| 10 | 9 | TA 5 post | 200 | 20 | 10 | prepared in | |
| | | ABT | (ABT100) | | | 0.5% (wt/vol) | |
| | | ABTgiven 2 | | | | methylcellulose | |
| | | hours prior | | | | (400 cP) | |
| | | to TA | | | | | |

Whole blood samples (0.1 mL each) were collected at the first time point via submandibular vein (SMV), or an appropriate peripheral vein if it was necessary. Up to 0.5 mL of whole blood was collected at terminal blood collection via intra cardiac puncture (ICP). For animals 1-3 from each of the 10 Groups, blood samples were collected at 0.5 and 4 hours after oral dosing. For animals 4-6 from each of the 10 Groups, blood samples were collected at 2 and 8 hours after oral dosing. For animals 7-9 from each of the 10 Groups, blood samples were collected at 12 and 24 hours after oral dosing.

The plasma samples and dose formulation samples were analyzed by the Testing Facility to determine the concentration of the test article using LC-MS/MS. PK parameters were estimated from the plasma concentration-time data by standard non-compartmental methods utilizing suitable analysis software.

TABLE 2
pK Summary

| | ND-012101 | | ND-011176 | |
|---|---|---|---|---|
| | (200 mg/kg) | | (200 mg/kg) | |
| | Gr 5 | Gr 6 | Gr 9 | Gr 10 |
| pK parameters | 12101 | ABT + 12101 | 11176 | ABT + 11176 |
| AUC(0-x) (ng · hr/mL) | 24,955 | NA | 15,684 | NA |
| AUC(0-x)/Dose | 125 | NA | 78.4 | NA |
| (ng · hr/mL/mg/kg) | | | | |
| AUC(0-∞) (ng hr/mL) | 24,955 | NA | 15,684 | NA |
| AUC(0-∞)/Dose | 125 | NA | 78.4 | NA |
| (ng · hr/mL/mg/kg) | | | | |
| % AUC Extrap | 0.00 | NA | 0.00 | NA |
| Original Dose (mg/kg) | 200 | 200^{\hat{ }} | 200 | 200^{\hat{ }} |
| tmax (hr) | 1 | NA | 0.5 | NA |
| Cmax (ng/mL) | 19,245 | 48,360^{\hat{ }} | 6,732 | 44,802^{\hat{ }} |
| Cmax/Dose | 96 | NA | 34 | NA |
| (ng/mL/mg/kg) | | | | |
| t_{1/2} (hr) | 0.79 | NA | 1.70 | NA |
| Regression Points (hr) | 4, 8, 12 | NA | 4, 8, 12 | NA |

| | | |
|---|---|---|
| Relative Bioavailability | NA | NA |
| [(TA + ABT)/TA]* | | |

NR Not Reportable, the % AUC extrapolation was >30.0%.
NC Not Calculable, the slope of the termination phase was positive.
*calculated by SD. AUC(0-x) was used.
^{\hat{ }}Manually added from the TA concentration data

TABLE 3
Drug plasma concentration vs. time Gr. 5
Group 5
PO 200 mg/kg ND-012101
Assayed Concentrations (ng/mL)

| Time | Mouse*(1, | Mouse*(2, | Mouse*(3, | |
|---|---|---|---|---|
| Hours | 4, 7) | 5, 8) | 6, 9) | Average |
| 0.5000 | 14700 | 23100 | 19900 | 19233 |
| 2.00 | 7900 | 3080 | 4590 | 5190 |
| 4.0 | 1350 | 3490 | 966 | 1935 |
| 8.00 | 67.6 | 22.3 | 100 | 63 |
| 12 | 2.00 | 2.10 | 1.19 | 2 |
| 24 | BQL | BQL | BQL | |

*2 blood collections were performed in each of 3 animals. Please see Study Protocol for details. Average values are recommended to be used primarily.

TABLE 4
Drug plasma concentration vs. time Gr. 6
Group 6
PO 200 mg/kg ND-012101 + 100 mg/kg ABT
Assayed Concentrations (ng/mL)

| Time | Mouse*(1, | Mouse*(2, | Mouse*(3, | |
|---|---|---|---|---|
| Hours | 4, 7) | 5, 8) | 6, 9) | Average |
| 0.5000 | 23300 | 22700 | 19100 | 21700 |
| 2.00 | 29800 | 37700 | 35500 | 34333 |
| 4.0 | 48200 | 47200 | 35000 | 43467 |
| 5.00^{\hat{ }} | 49100^{\hat{ }} | 47600^{\hat{ }} | 53350^{\hat{ }} | 50017^{\hat{ }} |
| 8.00 | NS | NS | NS | NS |
| 12 | NS | NS | NS | NS |
| 24 | NS | NS | NS | NS |

Note:
All animals (except 1, 2, 3 which were euthanized after 4 h blood collection as scheduled) were euthanized due to AEs about 5 hours post-dose
NS: no sample
8 h and 12 h samples were collected before the euthanasia
^{\hat{ }}the values were the mean of the values from the “8” and “12” TP.
*2 blood collections were performed in each of 3 animals. Please see Study Protocol for details. Average values are recommended to be used primarily.

TABLE 5
Drug plasma concentration vs. time Gr. 9
Group 9
PO 200 mg/mL ND-011176
Assayed Concentrations (ng/mL)

| Time | Mouse*(1, | Mouse*(2, | Mouse*(3, | |
|---|---|---|---|---|
| Hour | 4, 7) | 5, 8) | 6, 9) | Average |
| 0.5000 | 7880 | 5140 | 7170 | 6730 |
| 2.00 | 8580 | 5280 | 4450 | 6103 |
| 4.0 | 861 | 432 | 507 | 600 |
| 8.00 | 110 | 25 | 88.2 | 74 |
| 12 | 40.1 | BQL | 28.8 | 34 |
| 24 | 8.09 | BQL | BQL | 8 |

*2 blood collections were performed in each of 3 animals. Please see Study Protocol for details. Average values are recommended to be used primarily.

TABLE 6
Drug plasma concentration vs. time Gr. 10
Group 10
PO 200 mg/kg ND-011176 + 100 mg/kg ABT
Assayed Concentrations (ng/mL)

| Time | Mouse*(1, | Mouse*(2, | Mouse*(3, | |
|---|---|---|---|---|
| Hour | 4, 7) | 5, 8) | 6, 9) | Average |
| 0.5000 | 19600 | 18100 | 22600 | 20100 |
| 2.00 | 36400 | 29300 | 38200 | 34633 |
| 4.0 | 54800 | 34700 | 44900 | 44800 |
| 5.00^{\hat{ }} | 39200^{\hat{ }} | 41600^{\hat{ }} | 44050^{\hat{ }} | 41617^{\hat{ }} |
| 8.00 | NS | NS | NS | NS |
| 12 | NS | NS | NS | NS |
| 24 | NS | NS | NS | |

Note:
All animals (except 1, 2, 3 which were euthanized after 4 h blood collection as scheduled) were euthanized due to AEs about 5 hours post-dose
NS: no sample
8 h and 12 h samples were collected before the euthanasia

PK conclusion: Superior in vivo PK was observed with deuterated compound with and without the addition of 1-aminobenzotriazole (ABT), a known cytochrome P450 inhibitor.

Acute Murine Infection Model

ND-011176 (IP 3, in reference) in PLoS One 2012; 7(12):e52951 was shown to be effective in an acute murine infection model of TB infection. In this model, ND-011176 (IP 3) was shown to have bacteriostatic behavior in vivo, demonstrating a 2 log cfu reduction with respect to a non-treated controls both at 300 and 500 mg/kg. No signs of toxicity were observed at any of the administered doses. Based upon the in vivo efficacy observed with ND-011176 (IP 3) the deuterated analog (ND-012101) with superior in vivo PK should have efficacy in the acute murine TB infection model with and without the addition of ABT.

Chronic Murine Infection Model

When evaluated in the “Chronic Balb/c mouse model” of M. tuberculosis infection by the method of Lenaerts, A. J., et. al. 2005. Preclinical testing of the nitroimidazopyran PA-824 for activity against M. tuberculosis in a series of in vitro and in vivo models. AAC. 49(6):2294-301. ND-012101 at 200 mg/kg and ND-012101(100 mg/kg)+ABT (100 mg/kg) both achieved 100% mouse survival after 4 weeks of oral dosing. Both dosing regimens showed bacteriostatic activity with <1 log CFU drop; consistent with the efficacy observed with other aa3-type cytochrome c oxidase inhibitors within the chronic model of infection (see, ACS Infect. Dis. 2019, 5, 239-249). Organ analysis showed similar results to that of Isoniazid (INH) mono-therapy.

Gross Necropsy Observations

TABLE 7
Gross necropsy observations after 4 weeks
of treatment: Initial experimental design

| | | | | | | |
|---|---|---|---|---|---|---|
| Group | Treatment | Mouse | Weight | Lung | Spleen | notes |

| | | | | | |
|---|---|---|---|---|---|
| 3 | ND-012101 + ABT | A | 20.4 | + | Slightly enlarged |
| | | B | 20.0 | + | Slightly enlarged |
| | | C | 20.5 | + | Slightly enlarged |
| | | D | 18.6 | + | Slightly enlarged |
| | | E | 20.4 | + | Slightly enlarged |
| | | F | 20.8 | + | Slightly enlarged |
| 6 | ND-012101 | A | 21.5 | ++ | Slightly enlarged |
| | | B | 20.7 | ++ | Slightly enlarged |
| | | C | 20.3 | + | normal |
| | | D | 19.9 | ++ | Slightly enlarged |
| | | E | 20.8 | ++ | Slightly enlarged |
| | | F | 21.5 | ++ | Slightly enlarged |
| 8 | INH | A | 19.6 | + | enlarged |
| | | B | 20.2 | + | enlarged |
| | | C | 20.1 | + | enlarged |
| | | D | 19.9 | + | enlarged |
| | | E | 19.2 | + | enlarged |
| | | F | 20.8 | + | enlarged |
| 9 | CTL | A | 22.3 | +++ | enlarged |
| | | B | 21.8 | +++ | enlarged |
| | | C | 22.2 | +++ | enlarged |
| | | D | 20.4 | +++ | enlarged |
| | | E | 19.7 | +++ | enlarged |
| | | F | 19.0 | +++ | enlarged |

TABLE 8
Log10 CFU data

| | | Log | | | Log | |
|---|---|---|---|---|---|---|
| | | reduction | | | reduction | |
| | Lung | vs CTL | | Spleen | vs CTL | |
| Group | Log10 CFU ± SEM | (Lg) | *n | Log10 CFU ± SEM | (Sp) | *n |
| Day 21 | 5.87 ± 0.10 | | 5/5 | 4.35 ± 0.18 | | 5/5 |
| Pretreatment | | | | | | |
| Control | | | | | | |
| 4 Week | | | | | | |
| Treatment | | | | | | |
| CTL | 5.79 ± 0.12 | | 3/6 | 5.00 ± 0.13 | | 3/6 |
| ND-012101 (100) + | 5.62 ± 0.06 | 0.17 | 6/6 | 4.80 ± 0.14 | 0.20 | 6/6 |
| ABT (100) | | | | | | |
| ND-012101 (200) | 5.93 ± 0.08 | −0.14 | 6/6 | 5.02 ± 0.08 | −0.02 | 6/6 |
| INH (25) | 4.06 ± 0.11 | 1.73 | 6/6 | 2.13 ± 0.12^{a} | 2.87 | 5/6 |

The compounds of the present invention have not only the above described activity but also usefulness as a medicine, and have any or all of the following superior features:

a) exhibit high solubility,

b) having less risk of phototoxicity,

c) having less risk of hepatotoxicity,

d) having less risk of kidney toxicity,

e) having less risk of gastrointestinal disorders,

f) having less risk of drug interaction,

g) having high oral absorbability,

h) having small clearance,

i) having high distribution to a targeted tissue,

j) having intense enzymatic activity,

k) causing less induction of drug-metabolizing enzyme, and

l) having intense efficacy.

The contents of each patent, publication, citation, webpage, or other reference mentioned herein are hereby independently incorporated by reference.

1. A compound of the formula (A) or (A-Deut): 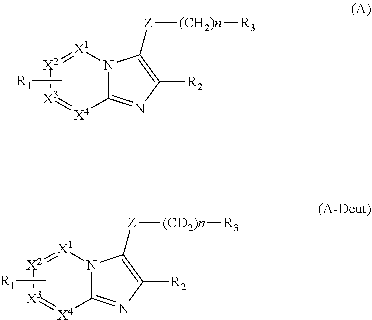
or salt thereof;
wherein:
X¹, X², X³, and X⁴ are independently C-D, CR₄, or N or a combination thereof;
Z is independently —C(O)NH—, —NHC(O)—; —C(O)O—, —C(O)C(O)—, —CH₂C(O)—, —CD₂C(O)—, —C(O)CH₂—, —C(O)CD₂-, or —NH—C(O)NH—;
n is an integer of 0 to 4;
each R₁ is independently D, CD₃, —OCD₃, Cl, Br, CF₃, SF₅, SF₃, F, —P(O)(CH₃)₂, SO₂CH₃, alkyl, deuterated alkyl, heterocycle, deuterated heterocycle, methoxy, alkoxy, deuterated alkoxy, aryl, deuterated aryl, heteroaryl, deuterated heteroaryl, amine, substituted amine, or deuterated amine;
R₂ is independently CD₃, CH₂CD₃, CH₃, —CD₂CD₃, ethyl, extended alkyl, deuterated alkyl, heterocycle, deuterated heterocycle, alkoxy, deuterated alkoxy, aryl, deuterated aryl, heteroaryl,
deuterated heteroaryl, deuterated amine, cycloalkyl, deuterated cycloalkyl;
R₃ is independently H, D, alkyl, deuterated alkyl, alkoxy, deuterated alkoxy, amino, alkyl amino, deuterated alkyl amino, cycloalkyl, deuterated cycloalkyl, heterocycle, deuterated heterocycle, aryl, deuterated aryl, aryloxy, or deuterated aryloxy; and
each R₄ is independently H, D, alkyl, deuterated alkyl, alkoxy, deuterated alkoxy, amino, alkyl amino, deuterated alkyl amino, cycloalkyl, deuterated cycloalkyl, heterocycle, deuterated heterocycle, aryl, deuterated aryl, aryloxy, deuterated aryloxy, halo, SF₅, SF₃, —P(O)(CH₃)₂, SO₂CH₃, or R₁;
and wherein in (A), at least one of R₁, R₂, R₃, or R₄ is deuterated.

2. A compound of claim 1, having the formula (A), or salt thereof.

3. A compound of claim 1, having the formula (A-Deut), or salt thereof.

4. A compound of the formula (B) or (B-Deut): 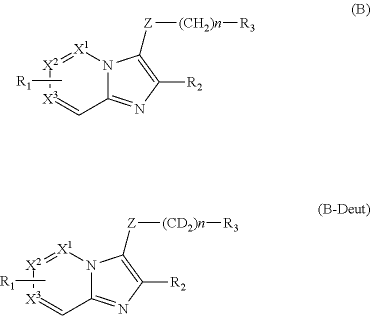
or salt thereof;
wherein:
X¹, X², and X³ are independently C-D, CR₄, or N or a combination thereof;
Z is independently —C(O)NH—, —NHC(O)—; —C(O)O—, —C(O)C(O)—, —CH₂C(O)—, —CD₂C(O)—, —C(O)CH₂—, —C(O)CD₂-, or —NH—C(O)NH—;
n is an integer of 0 to 4;
each R₁ is independently D, CD₃, —OCD₃, Cl, Br, CF₃, SF₅, SF₃, F, —P(O)(CH₃)₂, SO₂CH₃, alkyl, deuterated alkyl, heterocycle, deuterated heterocycle, methoxy, alkoxy, deuterated alkoxy, aryl, deuterated aryl, heteroaryl, deuterated heteroaryl, amine, substituted amine, or deuterated amine;
R₂ is independently CD₃, CH₂CD₃, CH₃, —CD₂CD₃, ethyl, extended alkyl, deuterated alkyl, heterocycle, deuterated heterocycle, alkoxy, deuterated alkoxy, aryl, deuterated aryl, heteroaryl, deuterated heteroaryl, deuterated amine, cycloalkyl, deuterated cycloalkyl;
R₃ is independently H, D, alkyl, deuterated alkyl, alkoxy, deuterated alkoxy, amino, alkyl amino, deuterated alkyl amino, cycloalkyl, deuterated cycloalkyl, heterocycle, deuterated heterocycle, aryl, deuterated aryl, aryloxy, or deuterated aryloxy; and
each R₄ is independently H, D, alkyl, deuterated alkyl, alkoxy, deuterated alkoxy, amino, alkyl amino, deuterated alkyl amino, cycloalkyl, deuterated cycloalkyl, heterocycle, deuterated heterocycle, aryl, deuterated aryl, aryloxy, deuterated aryloxy, halo, SF₅, SF₃, —P(O)(CH₃)₂, SO₂CH₃, or R₁;
and wherein in (B), at least one or more of R₁, R₂, R₃, or R₄ is deuterated.

5. A compound of claim 4, having the formula (B), or salt thereof.

6. A compound of claim 4, having the formula (B-Deut), or salt thereof.

7. The compound of claim 1, with the specific structure shown in FIG. 1.

8. A composition, comprising a compound as claimed in claim 1 and a pharmaceutically acceptable carrier or diluent.

9. A method of treating a tuberculosis mycobacterial infection or non-tuberculosis mycobacterial infection in a subject, comprising administering to said subject a compound as claimed in claim 1.

10. A method of killing or inhibiting the growth of M. tuberculosis, M. avium, M. leprae, or M. ulcerans, or a combination thereof, in a subject, comprising administering to said subject a compound as claimed in claim 1.

11. (canceled)

12. A method of treating a tuberculosis mycobacterial infection or non-tuberculosis mycobacterial infection in a subject, comprising administering to said subject a composition as claimed in claim 8.

13. A method of killing or inhibiting the growth of M. tuberculosis, M. avium, M. leprae, or M. ulcerans, or a combination thereof, in a subject, comprising administering to said subject a composition as claimed in claim 8.

14. The compound of claim 4, with the specific structure shown in FIG. 1.

15. A composition, comprising a compound as claimed in claim 4 and a pharmaceutically acceptable carrier or diluent.

16. A method of treating a tuberculosis mycobacterial infection or non-tuberculosis mycobacterial infection in a subject, comprising administering to said subject a compound as claimed in claim 4.

17. A method of killing or inhibiting the growth of M. tuberculosis, M. avium, M. leprae, or M. ulcerans, or a combination thereof, in a subject, comprising administering to said subject a compound as claimed in claim 4.

18. A method of treating a tuberculosis mycobacterial infection or non-tuberculosis mycobacterial infection in a subject, comprising administering to said subject a composition as claimed in claim 15.

19. A method of killing or inhibiting the growth of M. tuberculosis, M. avium, M. leprae, or M. ulcerans, or a combination thereof, in a subject, comprising administering to said subject a composition as claimed in claim 15.
